SEQUENCE LISTING (121 sequences)

SEQ ID NO: 1 - protein, 84 aa, Homo sapiens
SVSEIQLMHNLGKHLNSMERVEWLRKKLQDVHNFVALGAPLAPRDAGSQRPRKKEDNVLVESHEKSLGEADKADVNVLTKAKSQ

SEQ ID NO: 2 - protein, 83 aa, Artificial Sequence
  Human PTH 1-83
SVSEIQLMHNLGKHLNSMERVEWLRKKLQDVHNFVALGAPLAPRDAGSQRPRKKEDNVLVESHEKSLGEADKADVNVLTKAKS

SEQ ID NO: 3 - protein, 82 aa, Artificial Sequence
  human PTH 1-82
SVSEIQLMHNLGKHLNSMERVEWLRKKLQDVHNFVALGAPLAPRDAGSQRPRKKEDNVLVESHEKSLGEADKADVNVLTKAK

SEQ ID NO: 4 - protein, 81 aa, Artificial Sequence
  human PTH 1-81
SVSEIQLMHNLGKHLNSMERVEWLRKKLQDVHNFVALGAPLAPRDAGSQRPRKKEDNVLVESHEKSLGEADKADVNVLTKA

SEQ ID NO: 5 - protein, 80 aa, Artificial Sequence
  human PTH 1-80
SVSEIQLMHNLGKHLNSMERVEWLRKKLQDVHNFVALGAPLAPRDAGSQRPRKKEDNVLVESHEKSLGEADKADVNVLTK

SEQ ID NO: 6 - protein, 79 aa, Artificial Sequence
  human PTH 1-79
SVSEIQLMHNLGKHLNSMERVEWLRKKLQDVHNFVALGAPLAPRDAGSQRPRKKEDNVLVESHEKSLGEADKADVNVLT

SEQ ID NO: 7 - protein, 78 aa, Artificial Sequence
  human PTH 1-78
SVSEIQLMHNLGKHLNSMERVEWLRKKLQDVHNFVALGAPLAPRDAGSQRPRKKEDNVLVESHEKSLGEADKADVNVL

SEQ ID NO: 8 - protein, 77 aa, Artificial Sequence
  human PTH 1-77
SVSEIQLMHNLGKHLNSMERVEWLRKKLQDVHNFVALGAPLAPRDAGSQRPRKKEDNVLVESHEKSLGEADKADVNV

SEQ ID NO: 9 - protein, 76 aa, Artificial Sequence
  human PTH 1-76
SVSEIQLMHNLGKHLNSMERVEWLRKKLQDVHNFVALGAPLAPRDAGSQRPRKKEDNVLVESHEKSLGEADKADVN

SEQ ID NO: 10 - protein, 75 aa, Artificial Sequence
  human PTH 1-75
SVSEIQLMHNLGKHLNSMERVEWLRKKLQDVHNFVALGAPLAPRDAGSQRPRKKEDNVLVESHEKSLGEADKADV

SEQ ID NO: 11 - protein, 74 aa, Artificial Sequence
  human PTH 1-74
SVSEIQLMHNLGKHLNSMERVEWLRKKLQDVHNFVALGAPLAPRDAGSQRPRKKEDNVLVESHEKSLGEADKAD

SEQ ID NO: 12 - protein, 73 aa, Artificial Sequence
  human PTH 1-73
SVSEIQLMHNLGKHLNSMERVEWLRKKLQDVHNFVALGAPLAPRDAGSQRPRKKEDNVLVESHEKSLGEADKA

SEQ ID NO: 13 - protein, 72 aa, Artificial Sequence
  human PTH 1-72
SVSEIQLMHNLGKHLNSMERVEWLRKKLQDVHNFVALGAPLAPRDAGSQRPRKKEDNVLVESHEKSLGEADK

SEQ ID NO: 14 - protein, 71 aa, Artificial Sequence
  human PTH 1-71
SVSEIQLMHNLGKHLNSMERVEWLRKKLQDVHNFVALGAPLAPRDAGSQRPRKKEDNVLVESHEKSLGEAD

SEQ ID NO: 15 - protein, 70 aa, Artificial Sequence
  human PTH 1-70
SVSEIQLMHNLGKHLNSMERVEWLRKKLQDVHNFVALGAPLAPRDAGSQRPRKKEDNVLVESHEKSLGEA

SEQ ID NO: 16 - protein, 69 aa, Artificial Sequence
  human PTH 1-69
SVSEIQLMHNLGKHLNSMERVEWLRKKLQDVHNFVALGAPLAPRDAGSQRPRKKEDNVLVESHEKSLGE

SEQ ID NO: 17 - protein, 68 aa, Artificial Sequence
  human PTH 1-68
SVSEIQLMHNLGKHLNSMERVEWLRKKLQDVHNFVALGAPLAPRDAGSQRPRKKEDNVLVESHEKSLG

SEQ ID NO: 18 - protein, 67 aa, Artificial Sequence
  human PTH 1-67
SVSEIQLMHNLGKHLNSMERVEWLRKKLQDVHNFVALGAPLAPRDAGSQRPRKKEDNVLVESHEKSL

SEQ ID NO: 19 - protein, 66 aa, Artificial Sequence
  human PTH 1-66
SVSEIQLMHNLGKHLNSMERVEWLRKKLQDVHNFVALGAPLAPRDAGSQRPRKKEDNVLVESHEKS

SEQ ID NO: 20 - protein, 65 aa, Artificial Sequence
  human PTH 1-65
SVSEIQLMHNLGKHLNSMERVEWLRKKLQDVHNFVALGAPLAPRDAGSQRPRKKEDNVLVESHEK

SEQ ID NO: 21 - protein, 64 aa, Artificial Sequence
  human PTH 1-64
SVSEIQLMHNLGKHLNSMERVEWLRKKLQDVHNFVALGAPLAPRDAGSQRPRKKEDNVLVESHE

SEQ ID NO: 22 - protein, 63 aa, Artificial Sequence
  human PTH 1-63
SVSEIQLMHNLGKHLNSMERVEWLRKKLQDVHNFVALGAPLAPRDAGSQRPRKKEDNVLVESH

SEQ ID NO: 23 - protein, 62 aa, Artificial Sequence
  human PTH 1-62
SVSEIQLMHNLGKHLNSMERVEWLRKKLQDVHNFVALGAPLAPRDAGSQRPRKKEDNVLVES

SEQ ID NO: 24 - protein, 61 aa, Artificial Sequence
  human PTH 1-61
SVSEIQLMHNLGKHLNSMERVEWLRKKLQDVHNFVALGAPLAPRDAGSQRPRKKEDNVLVE

SEQ ID NO: 25 - protein, 60 aa, Artificial Sequence
  human PTH 1-60
SVSEIQLMHNLGKHLNSMERVEWLRKKLQDVHNFVALGAPLAPRDAGSQRPRKKEDNVLV

SEQ ID NO: 26 - protein, 59 aa, Artificial Sequence
  human PTH 1-59
SVSEIQLMHNLGKHLNSMERVEWLRKKLQDVHNFVALGAPLAPRDAGSQRPRKKEDNVL

SEQ ID NO: 27 - protein, 58 aa, Artificial Sequence
  human PTH 1-58
SVSEIQLMHNLGKHLNSMERVEWLRKKLQDVHNFVALGAPLAPRDAGSQRPRKKEDNV

SEQ ID NO: 28 - protein, 57 aa, Artificial Sequence
  human PTH 1-57
SVSEIQLMHNLGKHLNSMERVEWLRKKLQDVHNFVALGAPLAPRDAGSQRPRKKEDN

SEQ ID NO: 29 - protein, 56 aa, Artificial Sequence
  human PTH 1-56
SVSEIQLMHNLGKHLNSMERVEWLRKKLQDVHNFVALGAPLAPRDAGSQRPRKKED

SEQ ID NO: 30 - protein, 55 aa, Artificial Sequence
  human PTH 1-55
SVSEIQLMHNLGKHLNSMERVEWLRKKLQDVHNFVALGAPLAPRDAGSQRPRKKE

SEQ ID NO: 31 - protein, 54 aa, Artificial Sequence
  human PTH 1-54
SVSEIQLMHNLGKHLNSMERVEWLRKKLQDVHNFVALGAPLAPRDAGSQRPRKK

SEQ ID NO: 32 - protein, 53 aa, Artificial Sequence
  human PTH 1-53
SVSEIQLMHNLGKHLNSMERVEWLRKKLQDVHNFVALGAPLAPRDAGSQRPRK

SEQ ID NO: 33 - protein, 52 aa, Artificial Sequence
  human PTH 1-52
SVSEIQLMHNLGKHLNSMERVEWLRKKLQDVHNFVALGAPLAPRDAGSQRPR

SEQ ID NO: 34 - protein, 51 aa, Artificial Sequence
  human PTH 1-51
SVSEIQLMHNLGKHLNSMERVEWLRKKLQDVHNFVALGAPLAPRDAGSQRP

SEQ ID NO: 35 - protein, 50 aa, Artificial Sequence
  human PTH 1-50
SVSEIQLMHNLGKHLNSMERVEWLRKKLQDVHNFVALGAPLAPRDAGSQR

SEQ ID NO: 36 - protein, 49 aa, Artificial Sequence
  human PTH 1-49
SVSEIQLMHNLGKHLNSMERVEWLRKKLQDVHNFVALGAPLAPRDAGSQ

SEQ ID NO: 37 - protein, 48 aa, Artificial Sequence
  human PTH 1-48
SVSEIQLMHNLGKHLNSMERVEWLRKKLQDVHNFVALGAPLAPRDAGS

SEQ ID NO: 38 - protein, 47 aa, Artificial Sequence
  human PTH 1-47
SVSEIQLMHNLGKHLNSMERVEWLRKKLQDVHNFVALGAPLAPRDAG

SEQ ID NO: 39 - protein, 46 aa, Artificial Sequence
  human PTH 1-46
SVSEIQLMHNLGKHLNSMERVEWLRKKLQDVHNFVALGAPLAPRDA

SEQ ID NO: 40 - protein, 45 aa, Artificial Sequence
  human PTH 1-45
SVSEIQLMHNLGKHLNSMERVEWLRKKLQDVHNFVALGAPLAPRD

SEQ ID NO: 41 - protein, 44 aa, Artificial Sequence
  human PTH 1-44
SVSEIQLMHNLGKHLNSMERVEWLRKKLQDVHNFVALGAPLAPR

SEQ ID NO: 42 - protein, 43 aa, Artificial Sequence
  human PTH 1-43
SVSEIQLMHNLGKHLNSMERVEWLRKKLQDVHNFVALGAPLAP

SEQ ID NO: 43 - protein, 42 aa, Artificial Sequence
  human PTH 1-42
SVSEIQLMHNLGKHLNSMERVEWLRKKLQDVHNFVALGAPLA

SEQ ID NO: 44 - protein, 41 aa, Artificial Sequence
  human PTH-41
SVSEIQLMHNLGKHLNSMERVEWLRKKLQDVHNFVALGAPL

SEQ ID NO: 45 - protein, 40 aa, Artificial Sequence
  human PTH 1-40
SVSEIQLMHNLGKHLNSMERVEWLRKKLQDVHNFVALGAP

SEQ ID NO: 46 - protein, 39 aa, Artificial Sequence
  human PTH 1-39
SVSEIQLMHNLGKHLNSMERVEWLRKKLQDVHNFVALGA

SEQ ID NO: 47 - protein, 38 aa, Artificial Sequence
  human PTH 1-38
SVSEIQLMHNLGKHLNSMERVEWLRKKLQDVHNFVALG

SEQ ID NO: 48 - protein, 37 aa, Artificial Sequence
  human PTH 1-37
SVSEIQLMHNLGKHLNSMERVEWLRKKLQDVHNFVAL

SEQ ID NO: 49 - protein, 36 aa, Artificial Sequence
  human PTH 1-36
SVSEIQLMHNLGKHLNSMERVEWLRKKLQDVHNFVA

SEQ ID NO: 50 - protein, 35 aa, Artificial Sequence
  human PTH 1-35
SVSEIQLMHNLGKHLNSMERVEWLRKKLQDVHNFV

SEQ ID NO: 51 - protein, 34 aa, Artificial Sequence
  human PTH 1-34
SVSEIQLMHNLGKHLNSMERVEWLRKKLQDVHNF

SEQ ID NO: 52 - protein, 33 aa, Artificial Sequence
  human PTH 1-33
SVSEIQLMHNLGKHLNSMERVEWLRKKLQDVHN

SEQ ID NO: 53 - protein, 32 aa, Artificial Sequence
  human PTH 1-32
SVSEIQLMHNLGKHLNSMERVEWLRKKLQDVH

SEQ ID NO: 54 - protein, 31 aa, Artificial Sequence
  human PTH 1-31
SVSEIQLMHNLGKHLNSMERVEWLRKKLQDV

SEQ ID NO: 55 - protein, 30 aa, Artificial Sequence
  human PTH 1-30
SVSEIQLMHNLGKHLNSMERVEWLRKKLQD

SEQ ID NO: 56 - protein, 29 aa, Artificial Sequence
  human PTH 1-29
SVSEIQLMHNLGKHLNSMERVEWLRKKLQ

SEQ ID NO: 57 - protein, 28 aa, Artificial Sequence
  human PTH 1-28
SVSEIQLMHNLGKHLNSMERVEWLRKKL

SEQ ID NO: 58 - protein, 27 aa, Artificial Sequence
  human PTH 1-27
SVSEIQLMHNLGKHLNSMERVEWLRKK

SEQ ID NO: 59 - protein, 26 aa, Artificial Sequence
  human PTH 1-26
SVSEIQLMHNLGKHLNSMERVEWLRK

SEQ ID NO: 60 - protein, 25 aa, Artificial Sequence
  human PTH 1-25
SVSEIQLMHNLGKHLNSMERVEWLR

SEQ ID NO: 61 - protein, 84 aa, Artificial Sequence; identical to SEQ ID NO: 1
  amidated human PTH 1-84
  MOD_RES (84)..(84): AMIDATION

SEQ ID NO: 62 - protein, 83 aa, Artificial Sequence; identical to SEQ ID NO: 2
  amidated human PTH 1-83
  MOD_RES (83)..(83): AMIDATION

SEQ ID NO: 63 - protein, 82 aa, Artificial Sequence; identical to SEQ ID NO: 3
  amidated human PTH 1-82
  MOD_RES (82)..(82): AMIDATION

SEQ ID NO: 64 - protein, 81 aa, Artificial Sequence; identical to SEQ ID NO: 4
  amidated human PTH 1-81
  MOD_RES (81)..(81): AMIDATION

SEQ ID NO: 65 - protein, 80 aa, Artificial Sequence; identical to SEQ ID NO: 5
  amidated human PTH 1-80
  MOD_RES (80)..(80): AMIDATION

SEQ ID NO: 66 - protein, 79 aa, Artificial Sequence; identical to SEQ ID NO: 6
  amidated human PTH 1-79
  MOD_RES (79)..(79): AMIDATION

SEQ ID NO: 67 - protein, 78 aa, Artificial Sequence; identical to SEQ ID NO: 7
  amidated human PTH 1-78
  MOD_RES (78)..(78): AMIDATION

SEQ ID NO: 68 - protein, 77 aa, Artificial Sequence; identical to SEQ ID NO: 8
  amidated human PTH 1-77
  MOD_RES (77)..(77): AMIDATION

SEQ ID NO: 69 - protein, 76 aa, Artificial Sequence; identical to SEQ ID NO: 9
  amidated human PTH 1-76
  MOD_RES (76)..(76): AMIDATION

SEQ ID NO: 70 - protein, 75 aa, Artificial Sequence; identical to SEQ ID NO: 10
  amidated human PTH 1-75
  MOD_RES (75)..(75): AMIDATION

SEQ ID NO: 71 - protein, 74 aa, Artificial Sequence; identical to SEQ ID NO: 11
  amidated human PTH 1-74
  MOD_RES (74)..(74): AMIDATION

SEQ ID NO: 72 - protein, 73 aa, Artificial Sequence; identical to SEQ ID NO: 12
  amidated human PTH 1-73
  MOD_RES (73)..(73): AMIDATION

SEQ ID NO: 73 - protein, 72 aa, Artificial Sequence; identical to SEQ ID NO: 13
  amidated human PTH 1-72
  MOD_RES (72)..(72): AMIDATION

SEQ ID NO: 74 - protein, 71 aa, Artificial Sequence; identical to SEQ ID NO: 14
  amidated human PTH 1-71
  MOD_RES (71)..(71): AMIDATION

SEQ ID NO: 75 - protein, 70 aa, Artificial Sequence; identical to SEQ ID NO: 15
  amidated human PTH 1-70
  MOD_RES (70)..(70): AMIDATION

SEQ ID NO: 76 - protein, 69 aa, Artificial Sequence; identical to SEQ ID NO: 16
  amidated human PTH 1-69
  MOD_RES (69)..(69): AMIDATION

SEQ ID NO: 77 - protein, 68 aa, Artificial Sequence; identical to SEQ ID NO: 17
  amidated human PTH 1-68
  MOD_RES (68)..(68): AMIDATION

SEQ ID NO: 78 - protein, 67 aa, Artificial Sequence; identical to SEQ ID NO: 18
  amidated human PTH 1-67
  MOD_RES (67)..(67): AMIDATION

SEQ ID NO: 79 - protein, 66 aa, Artificial Sequence; identical to SEQ ID NO: 19
  amidated human PTH 1-66
  MOD_RES (66)..(66): AMIDATION

SEQ ID NO: 80 - protein, 65 aa, Artificial Sequence; identical to SEQ ID NO: 20
  amidated human PTH 1-65
  MOD_RES (65)..(65): AMIDATION

SEQ ID NO: 81 - protein, 64 aa, Artificial Sequence; identical to SEQ ID NO: 21
  amidated human PTH 1-64
  MOD_RES (64)..(64): AMIDATION

SEQ ID NO: 82 - protein, 63 aa, Artificial Sequence; identical to SEQ ID NO: 22
  amidated human PTH 1-63
  MOD_RES (63)..(63): AMIDATION

SEQ ID NO: 83 - protein, 62 aa, Artificial Sequence; identical to SEQ ID NO: 23
  amidated human PTH 1-62
  MOD_RES (62)..(62): AMIDATION

SEQ ID NO: 84 - protein, 61 aa, Artificial Sequence; identical to SEQ ID NO: 24
  amidated human PTH 1-61
  MOD_RES (61)..(61): AMIDATION

SEQ ID NO: 85 - protein, 60 aa, Artificial Sequence; identical to SEQ ID NO: 25
  amidated human PTH 1-60
  MOD_RES (60)..(60): ACETYLATION

SEQ ID NO: 86 - protein, 59 aa, Artificial Sequence; identical to SEQ ID NO: 26
  amidated human PTH 1-59
  MOD_RES (59)..(59): AMIDATION

SEQ ID NO: 87 - protein, 58 aa, Artificial Sequence; identical to SEQ ID NO: 27
  amidated human PTH 1-58
  MOD_RES (58)..(58): AMIDATION

SEQ ID NO: 88 - protein, 57 aa, Artificial Sequence; identical to SEQ ID NO: 28
  amidated human PTH 1-57
  MOD_RES (57)..(57): AMIDATION

SEQ ID NO: 89 - protein, 56 aa, Artificial Sequence; identical to SEQ ID NO: 29
  amidated human PTH 1-56
  MOD_RES (56)..(56): AMIDATION

SEQ ID NO: 90 - protein, 55 aa, Artificial Sequence; identical to SEQ ID NO: 30
  amidated human PTH 1-55
  MOD_RES (55)..(55): AMIDATION

SEQ ID NO: 91 - protein, 54 aa, Artificial Sequence; identical to SEQ ID NO: 31
  amidated human PTH 1-54
  MOD_RES (54)..(54): AMIDATION

SEQ ID NO: 92 - protein, 53 aa, Artificial Sequence; identical to SEQ ID NO: 32
  amidated human PTH 1-53
  MOD_RES (53)..(53): AMIDATION

SEQ ID NO: 93 - protein, 52 aa, Artificial Sequence; identical to SEQ ID NO: 33
  amidated human PTH 1-52
  MOD_RES (52)..(52): AMIDATION

SEQ ID NO: 94 - protein, 51 aa, Artificial Sequence; identical to SEQ ID NO: 34
  amidated human PTH 1-51
  MOD_RES (51)..(51): AMIDATION

SEQ ID NO: 95 - protein, 50 aa, Artificial Sequence; identical to SEQ ID NO: 35
  amidated human PTH 1-50
  MOD_RES (50)..(50): AMIDATION

SEQ ID NO: 96 - protein, 49 aa, Artificial Sequence; identical to SEQ ID NO: 36
  amidated human PTH 1-49
  MOD_RES (49)..(49): AMIDATION

SEQ ID NO: 97 - protein, 48 aa, Artificial Sequence; identical to SEQ ID NO: 37
  amidated human PTH 1-48
  MOD_RES (48)..(48): AMIDATION

SEQ ID NO: 98 - protein, 47 aa, Artificial Sequence; identical to SEQ ID NO: 38
  amidated human PTH 1-47
  MOD_RES (47)..(47): AMIDATION

SEQ ID NO: 99 - protein, 46 aa, Artificial Sequence; identical to SEQ ID NO: 39
  amidated human PTH 1-46
  MOD_RES (46)..(46): AMIDATION

SEQ ID NO: 100 - protein, 45 aa, Artificial Sequence; identical to SEQ ID NO: 40
  amidated human PTH 1-45
  MOD_RES (45)..(45): AMIDATION

SEQ ID NO: 101 - protein, 44 aa, Artificial Sequence; identical to SEQ ID NO: 41
  amidated human PTH 1-44
  MOD_RES (44)..(44): AMIDATION

SEQ ID NO: 102 - protein, 43 aa, Artificial Sequence; identical to SEQ ID NO: 42
  amidated human PTH 1-43
  MOD_RES (43)..(43): AMIDATION

SEQ ID NO: 103 - protein, 42 aa, Artificial Sequence; identical to SEQ ID NO: 43
  amidated human PTH 1-42
  MOD_RES (42)..(42): AMIDATION

SEQ ID NO: 104 - protein, 41 aa, Artificial Sequence; identical to SEQ ID NO: 44
  amidated human PTH 1-41
  MOD_RES (41)..(41): AMIDATION

SEQ ID NO: 105 - protein, 40 aa, Artificial Sequence; identical to SEQ ID NO: 45
  amidated human PTH 1-40
  MOD_RES (40)..(40): AMIDATION

SEQ ID NO: 106 - protein, 39 aa, Artificial Sequence; identical to SEQ ID NO: 46
  amidated human PTH 1-39
  MOD_RES (39)..(39): AMIDATION

SEQ ID NO: 107 - protein, 38 aa, Artificial Sequence; identical to SEQ ID NO: 47
  amidated human PTH 1-38
  MOD_RES (38)..(38): AMIDATION

SEQ ID NO: 108 - protein, 37 aa, Artificial Sequence; identical to SEQ ID NO: 48
  amidated human PTH 1-37
  MOD_RES (37)..(37): AMIDATION

SEQ ID NO: 109 - protein, 36 aa, Artificial Sequence; identical to SEQ ID NO: 49
  amidated human PTH 1-36
  MOD_RES (36)..(36): AMIDATION

SEQ ID NO: 110 - protein, 35 aa, Artificial Sequence; identical to SEQ ID NO: 50
  amidated human PTH 1-35
  MOD_RES (35)..(35): AMIDATION

SEQ ID NO: 111 - protein, 34 aa, Artificial Sequence; identical to SEQ ID NO: 51
  amidated human PTH 1-34
  MOD_RES (34)..(34): AMIDATION

SEQ ID NO: 112 - protein, 33 aa, Artificial Sequence; identical to SEQ ID NO: 52
  amidated human PTH 1-33
  MOD_RES (33)..(33): AMIDATION

SEQ ID NO: 113 - protein, 32 aa, Artificial Sequence; identical to SEQ ID NO: 53
  amidated human PTH 1-32
  MOD_RES (32)..(32): AMIDATION

SEQ ID NO: 114 - protein, 31 aa, Artificial Sequence; identical to SEQ ID NO: 54
  amidated human PTH 1-31
  MOD_RES (31)..(31): AMIDATION

SEQ ID NO: 115 - protein, 30 aa, Artificial Sequence; identical to SEQ ID NO: 55
  amidated human PTH 1-30
  MOD_RES (30)..(30): AMIDATION

SEQ ID NO: 116 - protein, 29 aa, Artificial Sequence
  amidated human PTH 1-29
  MOD_RES (29)..(29): AMIDATION
SVSEIQLMHNLGKHLNSMERVEWLRKKLQ

SEQ ID NO: 117 - protein, 28 aa, Artificial Sequence
  amidated human PTH 1-28
  MOD_RES (28)..(28): AMIDATION
SVSEIQLMHNLGKHLNSMERVEWLRKKL

SEQ ID NO: 118 - protein, 27 aa, Artificial Sequence
  amidated human PTH 1-27
  MOD_RES (27)..(27): AMIDATION
SVSEIQLMHNLGKHLNSMERVEWLRKK

SEQ ID NO: 119 - protein, 26 aa, Artificial Sequence
  amidated human PTH 1-26
  MOD_RES (26)..(26): AMIDATION
SVSEIQLMHNLGKHLNSMERVEWLRK

SEQ ID NO: 120 - protein, 25 aa, Artificial Sequence
  amidated human PTH 1-25
  MOD_RES (25)..(25): AMIDATION
SVSEIQLMHNLGKHLNSMERVEWLR

SEQ ID NO: 121 - protein, 141 aa, Homo sapiens
AVSEHQLLHDKGKSIQDLRRRFFLHHLIAEIHTAEIRATSEVSPNSKPSPNTKNHPVRFGSDDEGRYLTQETNKVETYKEQPLKTPGKKKKGKPGKRKEQEKKKRRTRSAWLDSGVTGSGLEGDHLSDTSTTSLELDSRRH

(19) United States
(12) Patent Application Publication
(10) Pub. No.: US 2023/0042670 A1
(43) Pub. Date: Feb. 9, 2023

(54) Hypoparathyroidism Treatment

(71) Applicant: Ascendis Pharma Bone Diseases A/S, Hellerup (DK)

(72) Inventor: Kennett SPROGØE, Hellerup (DK)

(21) Appl. No.: 17/783,640

(22) PCT Filed: Jan. 12, 2021
(86) PCT No.: PCT/EP2021/050460

§ 371 Date: Jun. 8, 2022

(30) Foreign Application Priority Data
Jan. 13, 2020 (EP) 20151350.4
Aug. 25, 2020 (EP) 20192565.8
Dec. 21, 2020 (EP) 20216065.1

(51) Int. Cl. A61K 47/34; A61K 47/22; A61P 5/18

(52) U.S. Cl. CPC A61K 47/34; A61K 47/22; A61P 5/18

(57) ABSTRACT

The present invention relates to a PTH compound for use in the treatment of hypoparathyroidism, wherein the treatment comprises single daily administrations of the PTH compound to a patient and titrating the patient off of standard of care within four weeks from the time the first dose of the PTH compound was administered.

The present invention relates to a PTH compound for use in the treatment of hypoparathyroidism, wherein the treatment comprises single daily administrations of the PTH compound to a patient and titrating the patient off of standard of care within four weeks from the time the first dose of the PTH compound was administered.

PTH regulates the body's extracellular calcium level within a very narrow range as well as phosphate homeostasis and bone turnover. Hypoparathyroidism (HP) is a rare disease of impaired PTH production. The majority of cases (75-78%) are acquired, occurring secondary to anterior neck surgery (generally thyroidectomy) in which the parathyroid glands are inadvertently damaged or removed. Among patients with chronic HP after total thyroidectomy, the risk of death over approximately a 4-year follow-up is 2-fold higher compared to patients without HP (Almquist, M., et al., Mortality in patients with permanent hypoparathyroidism after total thyroidectomy. Br J Surg, 2018. 105(10): p. 1313-1318). When serum calcium (sCa) levels drop without compensatory PTH secretion, renal reabsorption of calcium and excretion of phosphate decreases. In addition, conversion of 25-hydroxy vitamin D to active vitamin D by the kidneys also lessens. Lack of active vitamin D leads to reduced calcium and phosphate absorption by the small intestines, and lack of PTH leads to decreased bone turnover. The net result is that patients with untreated HP develop hypocalcemia, hyperphosphatemia, and increased urinary calcium excretion along with overly mineralized bone.

Standard-of-care (SOC) for chronic HP—specifically high dose active vitamin D and calcium—only corrects hypocalcemia, targeting a sCa just below or in the lower level of normal range to avoid worsening hypercalciuria, and is frequently associated with hypocalcemia prior to the next dose. Doses of active vitamin D and calcium often exceed 3.0 mcg calcitriol and 3000 mg calcium, the former usually taken 2 to 3 times per day and the latter often taken 4 to 6 times per day. Long-term high dose active vitamin D and calcium may produce adverse effects beyond the original problems associated with HP, including an increase in calcium x phosphate product and urinary calcium (uCa) that together may lead to nephrocalcinosis, nephrolithiasis, and renal insufficiency as well as ectopic calcifications. It would therefore greatly benefit patients if calcium homeostasis could be maintained in the absence of SOC, such as providing continuous physiological levels of PTH and maintaining a daily dietary intake with or without nutritional supplementation of Ca to achieve the recommended daily intake of 1000 to 1200 mg/day for the healthy population. This level of nutritional supplementation is required to maintain total body calcium homeostasis and balance, so that skeletal reservoirs of calcium are not depleted over the long-term to subsidize serum calcium. This recommended daily intake is what is necessary to replenish the calcium excreted daily through the kidneys and gastrointestinal tract. Some healthy individuals are able to achieve the recommended intake by diet, ie, food sources, alone; others—due to dietary preferences, or individual tolerance eg, dairy intolerance—must rely on calcium supplements in addition to food sources to approach or achieve 1000 to 1200 mg/day. In North America, it is estimated that adult men and women in the 25^{th} and 50^{th} percentiles for dietary, i.e. originating from food sources, calcium intake ingest 600-800 mg/day by diet and thus require an additional 400 to 600 mg/day by calcium tablet supplementation. 600 mg is proposed as the threshold because calcium carbonate tablets are commonly available and purchased in the 600 mg strength. Thus, calcium 600 mg per day would align with a single tablet of calcium per day.

According to literature from the US and Europe, 600 mg is considered an insufficient dose for the treatment of moderate-to-severe biochemical hypoparathyroidism. Instead, a typical calcium dose for the treatment of hypoparathyroidism is 1500 to 2000 mg per day. Thus, in order to meet recommended dietary intake of calcium, calcium supplements ≤600 mg/day as a nutritional supplement for the sake of reaching the recommended dietary intake are not considered treatment related.

Achieving the goal of calcium homeostasis in the absence of SOC has been attempted by administering short acting PTH molecules. Both PTH(1-34) and PTH(1-84) are approved drugs for osteoporosis and hypoparathyroidism, respectively. Both have been used clinically to treat hypoparathyroidism, but have failed to adequately address the disease, in part due to inadequate PTH activity throughout the day. In a double-blind, placebo-controlled, randomized phase 3 study in patients with hypoparathyroidism, patients were randomized to 50 μg per day of PTH(1-84) with a half-life ˜3 hours, or placebo for 24 weeks. Active vitamin D and calcium were progressively reduced, while rhPTH(1-84) could be titrated up from 50 μg to 75 μg and then 100 μg during a titration period of 5 weeks. The primary endpoint was the proportion of patients at week 24 who achieved a 50% or greater reduction from baseline in their daily dose of oral calcium and active vitamin D while maintaining a serum calcium concentration at or slightly below the lower limit of normal (ClinicalTrials.gov, number NCT00732615). In this study, only 53% patients in the rhPTH(1-84) group achieved the primary endpoint. However, there was no significant difference observed in urinary calcium excretion, or in clinical episodes of hypocalcemia, whereas clinical episodes of hypercalcemia showed a numerical increase on treatment.

In summary, there is a need for a more convenient and safer treatment of hypoparathyroidism with reduced side-effects.

It is therefore an object of the present invention to at least partially overcome the shortcomings described above.

This object is achieved with a PTH compound for use in the treatment of hypoparathyroidism, wherein the treatment comprises single daily administrations of the PTH compound to a patient and titrating the patient off of standard of care within four weeks from the time the first dose of the PTH compound was administered.

In another aspect the present invention relates to a method of treating or controlling a patient, wherein the patient is in need of the treatment of hypoparathyroidism, the method comprising the step of administering to said patient single daily administrations of a PTH compound and titrating the patient off of standard of care within four weeks from the time the first dose of the PTH compound was administered.

It was surprisingly found, that daily administration of a PTH compound, in particular a long-acting PTH preparation could restore serum calcium levels to normal levels (8.3 to 10.6 mg/dL or 2.075 to 2.65 mmol/L) and enabled withdrawal of SOC within just 28 days of starting the PTH therapy.

Within the present invention the terms are used having the meaning as follows.

As used herein the term “standard of care” or “SOC” refers to oral administration of calcium und active vitamin D.

As used herein the phrase “titrating off of standard of care” refers to removing oral calcium and active vitamin D supplementation in case of a daily nutritional calcium uptake of >750 mg/day and in case of a daily nutritional uptake of ≤750 mg/day refers to the removal of oral active vitamin D administration and a reduction in calcium supplement to ≤1000 mg/day, in certain embodiments to ≤630 mg/day, in certain embodiments to ≤500 mg/day while maintaining normal serum calcium levels (8.3 to 10.6 mg/dL or 2.075 to 2.65 mmol/L).

As used herein the terms “within normal level” and “within the normal range” with regard to serum calcium levels refer to the calcium level ordinarily found in a subject of a given species, sex and age, provided as the range given by the lower limit of normal and the upper limit of normal. In humans, the normal level in certain embodiments corresponds to a serum calcium level of above 8.5 mg/dL (albumin-adjusted). In humans the upper limit of normal is 10.5 mg/dL As used herein the term “serum calcium above 8.5 mg/dL” refers to albumin-adjusted calcium concentrations.

As used herein the term “albumin-adjusted” with regard to calcium levels means that the measured serum calcium level is corrected for calcium bound to albumin according to the following formula:

albumin-adjusted serum calcium (mg/dL)=measured total Ca (mg/dL)+0.8 (4.0−serum albumin [g/dL]).

As used herein the term “controlled-release PTH compound” refers to any compound, conjugate, crystal or admixture that comprises at least one PTH molecule or PTH moiety and from which the at least one PTH molecule or PTH moiety is released with a release half-life of at least 12 hours. In certain embodiments the release half-life is no more than 1 month. In certain embodiments the release half-life is no more than three weeks. In certain embodiments the release half-life is no more than two weeks. In certain embodiments the release half-life is no more than one week.

As used herein the terms “release half-life” and “half-life” refer to the time required under physiological conditions (i.e. aqueous buffer, pH 7.4, 37° C.) until half of all PTH or PTH moieties, respectively, of a PTH compound, in particular of a controlled-release PTH compound, are released from said controlled-release PTH compound.

As used herein the term “PTH” refers all PTH polypeptides, in certain embodiments from mammalian species, in certain embodiments from human and mammalian species, in certain embodiments from human and murine species, as well as their variants, analogs, orthologs, homologs, and derivatives and fragments thereof, that are characterized by raising serum calcium and renal phosphorus excretion, and lowering serum phosphorus and renal calcium excretion. The term “PTH” also refers to all PTH-related polypeptides (PTHrP), such as the polypeptide of SEQ ID NO:121, that bind to and activate the common PTH/PTHrP1 receptor. In certain embodiments the term “PTH” refers to the PTH polypeptide of SEQ ID NO:51 as well as its variants, homologs and derivatives exhibiting essentially the same biological activity, i.e. raising serum calcium and renal phosphorus excretion, and lowering serum phosphorus and renal calcium excretion.

In certain embodiments the term “PTH” refers to the following sequences:

(PTH 1-84)
SEQ ID NO: 1
SVSEIQLMHNLGKHLNSMERVEWLRKKLQDVHNFVALGAPLAPRDAGSQRPRKKED
NVLVESHEKSLGEADKADVNVLTKAKSQ
(PTH 1-83)
SEQ ID NO: 2
SVSEIQLMHNLGKHLNSMERVEWLRKKLQDVHNFVALGAPLAPRDAGSQRPRKKED
NVLVESHEKSLGEADKADVNVLTKAKS
(PTH 1-82)
SEQ ID NO: 3
SVSEIQLMHNLGKHLNSMERVEWLRKKLQDVHNFVALGAPLAPRDAGSQRPRKKED
NVLVESHEKSLGEADKADVNVLTKAK
(PTH 1-81)
SEQ ID NO: 4
SVSEIQLMHNLGKHLNSMERVEWLRKKLQDVHNFVALGAPLAPRDAGSQRPRKKED
NVLVESHEKSLGEADKADVNVLTKA
(PTH 1-80)
SEQ ID NO: 5
SVSEIQLMHNLGKHLNSMERVEWLRKKLQDVHNFVALGAPLAPRDAGSQRPRKKED
NVLVESHEKSLGEADKADVNVLTK
(PTH 1-79)
SEQ ID NO: 6
SVSEIQLMHNLGKHLNSMERVEWLRKKLQDVHNFVALGAPLAPRDAGSQRPRKKED
NVLVESHEKSLGEADKADVNVLT
(PTH 1-78)
SEQ ID NO: 7
SVSEIQLMHNLGKHLNSMERVEWLRKKLQDVHNFVALGAPLAPRDAGSQRPRKKED
NVLVESHEKSLGEADKADVNVL
(PTH 1-77)
SEQ ID NO: 8
SVSEIQLMHNLGKHLNSMERVEWLRKKLQDVHNFVALGAPLAPRDAGSQRPRKKED
NVLVESHEKSLGEADKADVNV
(PTH 1-76)
SEQ ID NO: 9
SVSEIQLMHNLGKHLNSMERVEWLRKKLQDVHNFVALGAPLAPRDAGSQRPRKKED
NVLVESHEKSLGEADKADVN
(PTH 1-75)
SEQ ID NO: 10
SVSEIQLMHNLGKHLNSMERVEWLRKKLQDVHNFVALGAPLAPRDAGSQRPRKKED
NVLVESHEKSLGEADKADV
(PTH 1-74)
SEQ ID NO: 11
SVSEIQLMHNLGKHLNSMERVEWLRKKLQDVHNFVALGAPLAPRDAGSQRPRKKED
NVLVESHEKSLGEADKAD
(PTH 1-73)
SEQ ID NO: 12
SVSEIQLMHNLGKHLNSMERVEWLRKKLQDVHNFVALGAPLAPRDAGSQRPRKKED
NVLVESHEKSLGEADKA
(PTH 1-72)
SEQ ID NO: 13
SVSEIQLMHNLGKHLNSMERVEWLRKKLQDVHNFVALGAPLAPRDAGSQRPRKKED
NVLVESHEKSLGEADK
(PTH 1-71)
SEQ ID NO: 14
SVSEIQLMHNLGKHLNSMERVEWLRKKLQDVHNFVALGAPLAPRDAGSQRPRKKED
NVLVESHEKSLGEAD
(PTH 1-70)
SEQ ID NO: 15
SVSEIQLMHNLGKHLNSMERVEWLRKKLQDVHNFVALGAPLAPRDAGSQRPRKKED
NVLVESHEKSLGEA
(PTH 1-69)
SEQ ID NO: 16
SVSEIQLMHNLGKHLNSMERVEWLRKKLQDVHNFVALGAPLAPRDAGSQRPRKKED
NVLVESHEKSLGE
(PTH 1-68)
SEQ ID NO: 17
SVSEIQLMHNLGKHLNSMERVEWLRKKLQDVHNFVALGAPLAPRDAGSQRPRKKED
NVLVESHEKSLG
(PTH 1-67)
SEQ ID NO: 18
SVSEIQLMHNLGKHLNSMERVEWLRKKLQDVHNFVALGAPLAPRDAGSQRPRKKED
NVLVESHEKSL
(PTH 1-66)
SEQ ID NO: 19
SVSEIQLMHNLGKHLNSMERVEWLRKKLQDVHNFVALGAPLAPRDAGSQRPRKKED
NVLVESHEKS
(PTH 1-65)
SEQ ID NO: 20
SVSEIQLMHNLGKHLNSMERVEWLRKKLQDVHNFVALGAPLAPRDAGSQRPRKKED
NVLVESHEK
(PTH 1-64)
SEQ ID NO: 21
SVSEIQLMHNLGKHLNSMERVEWLRKKLQDVHNFVALGAPLAPRDAGSQRPRKKED
NVLVESHE
(PTH 1-63)
SEQ ID NO: 22
SVSEIQLMHNLGKHLNSMERVEWLRKKLQDVHNFVALGAPLAPRDAGSQRPRKKED
NVLVESH
(PTH 1-62)
SEQ ID NO: 23
SVSEIQLMHNLGKHLNSMERVEWLRKKLQDVHNFVALGAPLAPRDAGSQRPRKKED
NVLVES
(PTH 1-61)
SEQ ID NO: 24
SVSEIQLMHNLGKHLNSMERVEWLRKKLQDVHNFVALGAPLAPRDAGSQRPRKKED
NVLVE
(PTH 1-60)
SEQ ID NO: 25
SVSEIQLMHNLGKHLNSMERVEWLRKKLQDVHNFVALGAPLAPRDAGSQRPRKKED
NVLV
(PTH 1-59)
SEQ ID NO: 26
SVSEIQLMHNLGKHLNSMERVEWLRKKLQDVHNFVALGAPLAPRDAGSQRPRKKEDNVL
(PTH 1-58)
SEQ ID NO: 27
SVSEIQLMHNLGKHLNSMERVEWLRKKLQDVHNFVALGAPLAPRDAGSQRPRKKEDNV
(PTH 1-57)
SEQ ID NO: 28
SVSEIQLMHNLGKHLNSMERVEWLRKKLQDVHNFVALGAPLAPRDAGSQRPRKKEDN
(PTH 1-56)
SEQ ID NO: 29
SVSEIQLMHNLGKHLNSMERVEWLRKKLQDVHNFVALGAPLAPRDAGSQRPRKKED
(PTH 1-55)
SEQ ID NO: 30
SVSEIQLMHNLGKHLNSMERVEWLRKKLQDVHNFVALGAPLAPRDAGSQRPRKKE
(PTH 1-54)
SEQ ID NO: 31
SVSEIQLMHNLGKHLNSMERVEWLRKKLQDVHNFVALGAPLAPRDAGSQRPRKK
(PTH 1-53)
SEQ ID NO: 32
SVSEIQLMHNLGKHLNSMERVEWLRKKLQDVHNFVALGAPLAPRDAGSQRPRK
(PTH 1-52)
SEQ ID NO: 33
SVSEIQLMHNLGKHLNSMERVEWLRKKLQDVHNFVALGAPLAPRDAGSQRPR
(PTH 1-51)
SEQ ID NO: 34
SVSEIQLMHNLGKHLNSMERVEWLRKKLQDVHNFVALGAPLAPRDAGSQRP
(PTH 1-50)
SEQ ID NO: 35
SVSEIQLMHNLGKHLNSMERVEWLRKKLQDVHNFVALGAPLAPRDAGSQR
(PTH 1-49)
SEQ ID NO: 36
SVSEIQLMHNLGKHLNSMERVEWLRKKLQDVHNFVALGAPLAPRDAGSQ
(PTH 1-48)
SEQ ID NO: 37
SVSEIQLMHNLGKHLNSMERVEWLRKKLQDVHNFVALGAPLAPRDAGS
(PTH 1-47)
SEQ ID NO: 38
SVSEIQLMHNLGKHLNSMERVEWLRKKLQDVHNFVALGAPLAPRDAG
(PTH 1-46)
SEQ ID NO: 39
SVSEIQLMHNLGKHLNSMERVEWLRKKLQDVHNFVALGAPLAPRDA
(PTH 1-45)
SEQ ID NO: 40
SVSEIQLMHNLGKHLNSMERVEWLRKKLQDVHNFVALGAPLAPRD
(PTH 1-44)
SEQ ID NO: 41
SVSEIQLMHNLGKHLNSMERVEWLRKKLQDVHNFVALGAPLAPR
(PTH 1-43)
SEQ ID NO: 42
SVSEIQLMHNLGKHLNSMERVEWLRKKLQDVHNFVALGAPLAP
(PTH 1-42)
SEQ ID NO: 43
SVSEIQLMHNLGKHLNSMERVEWLRKKLQDVHNFVALGAPLA
(PTH 1-41)
SEQ ID NO: 44
SVSEIQLMHNLGKHLNSMERVEWLRKKLQDVHNFVALGAPL
(PTH 1-40)
SEQ ID NO: 45
SVSEIQLMHNLGKHLNSMERVEWLRKKLQDVHNFVALGAP
(PTH 1-39)
SEQ ID NO: 46
SVSEIQLMHNLGKHLNSMERVEWLRKKLQDVHNFVALGA
(PTH 1-38)
SEQ ID NO: 47
SVSEIQLMHNLGKHLNSMERVEWLRKKLQDVHNFVALG
(PTH 1-37)
SEQ ID NO: 48
SVSEIQLMHNLGKHLNSMERVEWLRKKLQDVHNFVAL
(PTH 1-36)
SEQ ID NO: 49
SVSEIQLMHNLGKHLNSMERVEWLRKKLQDVHNFVA
(PTH 1-35)
SEQ ID NO: 50
SVSEIQLMHNLGKHLNSMERVEWLRKKLQDVHNFV
(PTH 1-34)
SEQ ID NO: 51
SVSEIQLMHNLGKHLNSMERVEWLRKKLQDVHNF
(PTH 1-33)
SEQ ID NO: 52
SVSEIQLMHNLGKHLNSMERVEWLRKKLQDVHN
(PTH 1-32)
SEQ ID NO: 53
SVSEIQLMHNLGKHLNSMERVEWLRKKLQDVH
(PTH 1-31)
SEQ ID NO: 54
SVSEIQLMHNLGKHLNSMERVEWLRKKLQDV
(PTH 1-30)
SEQ ID NO: 55
SVSEIQLMHNLGKHLNSMERVEWLRKKLQD
(PTH 1-29)
SEQ ID NO: 56
SVSEIQLMHNLGKHLNSMERVEWLRKKLQ
(PTH 1-28)
SEQ ID NO: 57
SVSEIQLMHNLGKHLNSMERVEWLRKKL
(PTH 1-27)
SEQ ID NO: 58
SVSEIQLMHNLGKHLNSMERVEWLRKK
(PTH 1-26)
SEQ ID NO: 59
SVSEIQLMHNLGKHLNSMERVEWLRK
(PTH 1-25)
SEQ ID NO: 60
SVSEIQLMHNLGKHLNSMERVEWLR
(amidated PTH 1-84)
SEQ ID NO: 61
SVSEIQLMHNLGKHLNSMERVEWLRKKLQDVHNFVALGAPLAPRDAGSQRPRKKED
NVLVESHEKSLGEADKADVNVLTKAKSQ;
wherein the C-terminus is amidated
(amidated PTH 1-83)
SEQ ID NO: 62
SVSEIQLMHNLGKHLNSMERVEWLRKKLQDVHNFVALGAPLAPRDAGSQRPRKKED
NVLVESHEKSLGEADKADVNVLTKAKS;
wherein the C-terminus is amidated
(amidated PTH 1-82)
SEQ ID NO: 63
SVSEIQLMHNLGKHLNSMERVEWLRKKLQDVHNFVALGAPLAPRDAGSQRPRKKED
NVLVESHEKSLGEADKADVNVLTKAK;
wherein the C-terminus is amidated
(amidated PTH 1-81)
SEQ ID NO: 64
SVSEIQLMHNLGKHLNSMERVEWLRKKLQDVHNFVALGAPLAPRDAGSQRPRKKED
NVLVESHEKSLGEADKADVNVLTKA;
wherein the C-terminus is amidated
(amidated PTH 1-80)
SEQ ID NO: 65
SVSEIQLMHNLGKHLNSMERVEWLRKKLQDVHNFVALGAPLAPRDAGSQRPRKKED
NVLVESHEKSLGEADKADVNVLTK;
wherein the C-terminus is amidated
(amidated PTH 1-79)
SEQ ID NO: 66
SVSEIQLMHNLGKHLNSMERVEWLRKKLQDVHNFVALGAPLAPRDAGSQRPRKKED
NVLVESHEKSLGEADKADVNVLT;
wherein the C-terminus is amidated
(amidated PTH 1-78)
SEQ ID NO: 67
SVSEIQLMHNLGKHLNSMERVEWLRKKLQDVHNFVALGAPLAPRDAGSQRPRKKED
NVLVESHEKSLGEADKADVNVL;
wherein the C-terminus is amidated
(amidated PTH 1-77)
SEQ ID NO: 68
SVSEIQLMHNLGKHLNSMERVEWLRKKLQDVHNFVALGAPLAPRDAGSQRPRKKED
NVLVESHEKSLGEADKADVNV;
wherein the C-terminus is amidated
(amidated PTH 1-76)
SEQ ID NO: 69
SVSEIQLMHNLGKHLNSMERVEWLRKKLQDVHNFVALGAPLAPRDAGSQRPRKKED
NVLVESHEKSLGEADKADVN;
wherein the C-terminus is amidated
(amidated PTH 1-75)
SEQ ID NO: 70
SVSEIQLMHNLGKHLNSMERVEWLRKKLQDVHNFVALGAPLAPRDAGSQRPRKKED
NVLVESHEKSLGEADKADV;
wherein the C-terminus is amidated
(amidated PTH 1-74)
SEQ ID NO: 71
SVSEIQLMHNLGKHLNSMERVEWLRKKLQDVHNFVALGAPLAPRDAGSQRPRKKED
NVLVESHEKSLGEADKAD;
wherein the C-terminus is amidated
(amidated PTH 1-73)
SEQ ID NO: 72
SVSEIQLMHNLGKHLNSMERVEWLRKKLQDVHNFVALGAPLAPRDAGSQRPRKKED
NVLVESHEKSLGEADKA;
wherein the C-terminus is amidated
(amidated PTH 1-72)
SEQ ID NO: 73
SVSEIQLMHNLGKHLNSMERVEWLRKKLQDVHNFVALGAPLAPRDAGSQRPRKKED
NVLVESHEKSLGEADK;
wherein the C-terminus is amidated
(amidated PTH 1-71)
SEQ ID NO: 74
SVSEIQLMHNLGKHLNSMERVEWLRKKLQDVHNFVALGAPLAPRDAGSQRPRKKED
NVLVESHEKSLGEAD;
wherein the C-terminus is amidated
(amidated PTH 1-70)
SEQ ID NO: 75
SVSEIQLMHNLGKHLNSMERVEWLRKKLQDVHNFVALGAPLAPRDAGSQRPRKKED
NVLVESHEKSLGEA;
wherein the C-terminus is amidated
(amidated PTH 1-69)
SEQ ID NO: 76
SVSEIQLMHNLGKHLNSMERVEWLRKKLQDVHNFVALGAPLAPRDAGSQRPRKKED
NVLVESHEKSLGE;
wherein the C-terminus is amidated
(amidated PTH 1-68)
SEQ ID NO: 77
SVSEIQLMHNLGKHLNSMERVEWLRKKLQDVHNFVALGAPLAPRDAGSQRPRKKED
NVLVESHEKSLG;
wherein the C-terminus is amidated
(amidated PTH 1-67)
SEQ ID NO: 78
SVSEIQLMHNLGKHLNSMERVEWLRKKLQDVHNFVALGAPLAPRDAGSQRPRKKED
NVLVESHEKSL;
wherein the C-terminus is amidated
(amidated PTH 1-66)
SEQ ID NO: 79
SVSEIQLMHNLGKHLNSMERVEWLRKKLQDVHNFVALGAPLAPRDAGSQRPRKKED
NVLVESHEKS;
wherein the C-terminus is amidated
(amidated PTH 1-65)
SEQ ID NO: 80
SVSEIQLMHNLGKHLNSMERVEWLRKKLQDVHNFVALGAPLAPRDAGSQRPRKKED
NVLVESHEK;
wherein the C-terminus is amidated
(amidated PTH 1-64)
SEQ ID NO: 81
SVSEIQLMHNLGKHLNSMERVEWLRKKLQDVHNFVALGAPLAPRDAGSQRPRKKED
NVLVESHE;
wherein the C-terminus is amidated
(amidated PTH 1-63)
SEQ ID NO: 82
SVSEIQLMHNLGKHLNSMERVEWLRKKLQDVHNFVALGAPLAPRDAGSQRPRKKED
NVLVESH;
wherein the C-terminus is amidated
(amidated PTH 1-62)
SEQ ID NO: 83
SVSEIQLMHNLGKHLNSMERVEWLRKKLQDVHNFVALGAPLAPRDAGSQRPRKKED
NVLVES;
wherein the C-terminus is amidated
(amidated PTH 1-61)
SEQ ID NO: 84
SVSEIQLMHNLGKHLNSMERVEWLRKKLQDVHNFVALGAPLAPRDAGSQRPRKKED
NVLVE;
wherein the C-terminus is amidated
(amidated PTH 1-60)
SEQ ID NO: 85
SVSEIQLMHNLGKHLNSMERVEWLRKKLQDVHNFVALGAPLAPRDAGSQRPRKKED
NVLV;
wherein the C-terminus is amidated
(amidated PTH 1-59)
SEQ ID NO: 86
SVSEIQLMHNLGKHLNSMERVEWLRKKLQDVHNFVALGAPLAPRDAGSQRPRKKED
NVL;
wherein the C-terminus is amidated
(amidated PTH 1-58)
SEQ ID NO: 87
SVSEIQLMHNLGKHLNSMERVEWLRKKLQDVHNFVALGAPLAPRDAGSQRPRKKEDNV;
wherein the C-terminus is amidated
(amidated PTH 1-57)
SEQ ID NO: 88
SVSEIQLMHNLGKHLNSMERVEWLRKKLQDVHNFVALGAPLAPRDAGSQRPRKKEDN;
wherein the C-terminus is amidated
(amidated PTH 1-56)
SEQ ID NO: 89
SVSEIQLMHNLGKHLNSMERVEWLRKKLQDVHNFVALGAPLAPRDAGSQRPRKKED;
wherein the C-terminus is amidated
(amidated PTH 1-55)
SEQ ID NO: 90
SVSEIQLMHNLGKHLNSMERVEWLRKKLQDVHNFVALGAPLAPRDAGSQRPRKKE;
wherein the C-terminus is amidated
(amidated PTH 1-54)
SEQ ID NO: 91
SVSEIQLMHNLGKHLNSMERVEWLRKKLQDVHNFVALGAPLAPRDAGSQRPRKK;
wherein the C-terminus is amidated
(amidated PTH 1-53)
SEQ ID NO: 92
SVSEIQLMHNLGKHLNSMERVEWLRKKLQDVHNFVALGAPLAPRDAGSQRPRK;
wherein the C-terminus is amidated
(amidated PTH 1-52)
SEQ ID NO: 93
SVSEIQLMHNLGKHLNSMERVEWLRKKLQDVHNFVALGAPLAPRDAGSQRPR;
wherein the C-terminus is amidated
(amidated PTH 1-51)
SEQ ID NO: 94
SVSEIQLMHNLGKHLNSMERVEWLRKKLQDVHNFVALGAPLAPRDAGSQRP;
wherein the C-terminus is amidated
(amidated PTH 1-50)
SEQ ID NO: 95
SVSEIQLMHNLGKHLNSMERVEWLRKKLQDVHNFVALGAPLAPRDAGSQR;
wherein the C-terminus is amidated
(amidated PTH 1-49)
SEQ ID NO: 96
SVSEIQLMHNLGKHLNSMERVEWLRKKLQDVHNFVALGAPLAPRDAGSQ;
wherein the C-terminus is amidated
(amidated PTH 1-48)
SEQ ID NO: 97
SVSEIQLMHNLGKHLNSMERVEWLRKKLQDVHNFVALGAPLAPRDAGS;
wherein the C-terminus is amidated
(amidated PTH 1-47)
SEQ ID NO: 98
SVSEIQLMHNLGKHLNSMERVEWLRKKLQDVHNFVALGAPLAPRDAG;
wherein the C-terminus is amidated
(amidated PTH 1-46)
SEQ ID NO: 99
SVSEIQLMHNLGKHLNSMERVEWLRKKLQDVHNFVALGAPLAPRDA;
wherein the C-terminus is amidated
(amidated PTH 1-45)
SEQ ID NO: 100
SVSEIQLMHNLGKHLNSMERVEWLRKKLQDVHNFVALGAPLAPRD;
wherein the C-terminus is amidated
(amidated PTH 1-44)
SEQ ID NO: 101
SVSEIQLMHNLGKHLNSMERVEWLRKKLQDVHNFVALGAPLAPR;
wherein the C-terminus is amidated
(amidated PTH 1-43)
SEQ ID NO: 102
SVSEIQLMHNLGKHLNSMERVEWLRKKLQDVHNFVALGAPLAP;
wherein the C-terminus is amidated
(amidated PTH 1-42)
SEQ ID NO: 103
SVSEIQLMHNLGKHLNSMERVEWLRKKLQDVHNFVALGAPLA;
wherein the C-terminus is amidated
(amidated PTH 1-41)
SEQ ID NO: 104
SVSEIQLMHNLGKHLNSMERVEWLRKKLQDVHNFVALGAPL;
wherein the C-terminus is amidated
(amidated PTH 1-40)
SEQ ID NO: 105
SVSEIQLMHNLGKHLNSMERVEWLRKKLQDVHNFVALGAP;
wherein the C-terminus is amidated
(amidated PTH 1-39)
SEQ ID NO: 106
SVSEIQLMHNLGKHLNSMERVEWLRKKLQDVHNFVALGA;
wherein the C-terminus is amidated
(amidated PTH 1-38)
SEQ ID NO: 107
SVSEIQLMHNLGKHLNSMERVEWLRKKLQDVHNFVALG;
wherein the C-terminus is amidated
(amidated PTH 1-37)
SEQ ID NO: 108
SVSEIQLMHNLGKHLNSMERVEWLRKKLQDVHNFVAL;
wherein the C-terminus is amidated
(amidated PTH 1-36)
SEQ ID NO: 109
SVSEIQLMHNLGKHLNSMERVEWLRKKLQDVHNFVA;
wherein the C-terminus is amidated
(amidated PTH 1-35)
SEQ ID NO: 110
SVSEIQLMHNLGKHLNSMERVEWLRKKLQDVHNFV;
wherein the C-terminus is amidated
(amidated PTH 1-34)
SEQ ID NO: 111
SVSEIQLMHNLGKHLNSMERVEWLRKKLQDVHNF;
wherein the C-terminus is amidated
(amidated PTH 1-33)
SEQ ID NO: 112
SVSEIQLMHNLGKHLNSMERVEWLRKKLQDVHN;
wherein the C-terminus is amidated
(amidated PTH 1-32)
SEQ ID NO: 113
SVSEIQLMHNLGKHLNSMERVEWLRKKLQDVH;
wherein the C-terminus is amidated
(amidated PTH 1-31)
SEQ ID NO: 114
SVSEIQLMHNLGKHLNSMERVEWLRKKLQDV;
wherein the C-terminus is amidated
(amidated PTH 1-30)
SEQ ID NO: 115
SVSEIQLMHNLGKHLNSMERVEWLRKKLQD;
wherein the C-terminus is amidated
(amidated PTH 1-29)
SEQ ID NO: 116
SVSEIQLMHNLGKHLNSMERVEWLRKKLQ;
wherein the C-terminus is amidated
(amidated PTH 1-28)
SEQ ID NO: 117
SVSEIQLMHNLGKHLNSMERVEWLRKKL;
wherein the C-terminus is amidated
(amidated PTH 1-27)
SEQ ID NO: 118
SVSEIQLMHNLGKHLNSMERVEWLRKK;
wherein the C-terminus is amidated
(amidated PTH 1-26)
SEQ ID NO: 119
SVSEIQLMHNLGKHLNSMERVEWLRK;
wherein the C-terminus is amidated
(amidated PTH 1-25)
SEQ ID NO: 120
SVSEIQLMHNLGKHLNSMERVEWLR;
wherein the C-terminus is amidated
(PTHrP)
SEQ ID NO: 121
AVSEHQLLHDKGKSIQDLRRRFFLHHLIAEIHTAEIRATSEVSPNSKPSPNTKNHPVRF
GSDDEGRYLTQETNKVETYKEQPLKTPGKKKKGKPGKRKEQEKKKRRTRSAWLDS
GVTGSGLEGDHLSDTSTTSLELDSRRH

In certain embodiments the term “PTH” refers to the sequence of SEQ ID:NOs 47, 48, 49, 50, 51, 52, 53, 54, 55, 107, 108, 109, 110, 111, 112, 113, 114 and 115. In certain embodiments the term “PTH” refers to the sequence of SEQ ID:NOs 50, 51, 52, 110, 111 and 112. In certain embodiments the term “PTH” refers to the sequence of SEQ ID NO:51.

As used herein, the term “PTH polypeptide variant” refers to a sequence from the same species that differs from a reference PTH or PTHrP sequence. In certain embodiments such reference is a PTH sequence and has the sequence of SEQ ID NO:51. Generally, differences are limited so that the amino acid sequence of the reference and the variant are closely similar overall and, in many regions, identical. In certain embodiments PTH variants are at least 70%, 80%, 90%, or 95% identical to a reference PTH or PTHrP, in certain embodiments to the PTH of SEQ ID NO:51. By a PTH or PTHrP having an amino acid sequence at least, for example, 95% “identical” to a query amino acid sequence, it is intended that said amino acid sequence is identical to the query sequence except that it may include up to five amino acid alterations per each 100 amino acids of the query amino acid sequence. These alterations of the reference sequence may occur at the amino (N-terminal) or carboxy terminal (C-terminal) positions of the reference amino acid sequence or anywhere between those terminal positions, interspersed either individually among residues in the reference sequence or in one or more contiguous groups within the reference sequence. The query sequence may be an entire amino acid sequence of the reference sequence or any fragment specified as described herein. In certain embodiments the query sequence is the sequence of SEQ ID NO:51.

Such PTH variants may be naturally occurring variants, such as naturally occurring allelic variants encoded by one of several alternate forms of a PTH or PTHrP occupying a given locus on a chromosome or an organism, or isoforms encoded by naturally occurring splice variants originating from a single primary transcript. Alternatively, a PTH variant may be a variant that is not known to occur naturally and that can be made by mutagenesis techniques known in the art.

It is known in the art that one or more amino acids may be deleted from the N-terminus or C-terminus of a bioactive protein or peptide without substantial loss of biological function. Such N- and/or C-terminal deletions are also encompassed by the term PTH variant.

It is also recognized by one of ordinary skill in the art that some amino acid sequences of PTH or PTHrP may be varied without significant effect of the structure or function of the PTH or PTHrP. Such mutants include deletions, insertions, inversions, repeats, and substitutions selected according to general rules known in the art so as to have little effect on activity. For example, guidance concerning how to make phenotypically silent amino acid substitutions is provided in Bowie et al. (1990), Science 247:1306-1310, which is hereby incorporated by reference in its entirety, wherein the authors indicate that there are two main approaches for studying the tolerance of the amino acid sequence to change.

The term PTH also encompasses all PTH and PTHrP sequences encoded by PTH and PTHrP analogs, orthologs, and/or species homologs. It is also recognized by one of ordinary skill in the art that PTHrP and PTHrP analogs bind to activate the common PTH/PTHrP1 receptor, so the term PTH sequence also encompasses all PTHrP analogs. As used herein, the term “PTH analog” refers to PTH and PTHrP of different and unrelated organisms which perform the same functions in each organism, but which did not originate from an ancestral structure that the organisms' ancestors had in common. Instead, analogous PTH and PTHrP arose separately and then later evolved to perform the same or similar functions. In other words, analogous PTH and PTHrP sequences are proteins or peptides with quite different amino acid sequences but that perform the same biological activity, namely raising serum calcium and renal phosphorus excretion, and lowering serum phosphorus and renal calcium excretion.

As used herein, the term “PTH homolog” refers to PTH and PTHrP of different organisms which perform the same functions in each organism, and which originate from an ancestral structure that the organisms' ancestors had in common. In other words, homologous PTH sequences are proteins or peptides with quite similar amino acid sequences that perform the same biological activity, namely raising serum calcium and renal phosphorus excretion, and lowering serum phosphorus and renal calcium excretion. In certain embodiments PTH homologs may be defined as proteins or peptides exhibiting at least 40%, 50%, 60%, 70%, 80%, 90% or 95% identity to a reference PTH or PTHrP protein or peptide, in certain embodiments the PTH sequence of SEQ ID NO:51.

Thus, a PTH according to the invention may be, for example: (i) one in which at least one of the amino acids residues is substituted with a conserved or non-conserved amino acid residue, in certain embodiments a conserved amino acid residue, and such substituted amino acid residue may or may not be one encoded by the genetic code; and/or (ii) one in which at least one of the amino acid residues includes a substituent group; and/or (iii) one in which the PTH sequence is fused with another compound, such as a compound to increase the half-life of the polypeptide (for example, polyethylene glycol); and/or (iv) one in which additional amino acids are fused to the PTH sequence, such as an IgG Fc fusion region peptide or protein or leader or secretory sequence or a sequence which is employed for purification of the above form of the protein or peptide or a pre-protein sequence.

As used herein, the term “PTH fragment” refers to any protein or peptide comprising a contiguous span of a part of the amino acid sequence of a PTH or PTHrP sequence, in certain embodiments the sequence of SEQ ID NO:51.

More specifically, a PTH fragment comprises at least 6, such as at least 8, at least 10 or at least 17 consecutive amino acids of a PTH or PTHrP sequence, in certain embodiments of the sequence of SEQ ID NO:51. A PTH fragment may additionally be described as sub-genuses of PTH or PTHrP sequences comprising at least 6 amino acids, wherein “at least 6” is defined as any integer between 6 and the integer representing the C-terminal amino acid of a PTH or PTHrP sequence, in certain embodiments of the sequence of SEQ ID No:51. Further included are species of PTH or PTHrP fragments of at least 6 amino acids in length, as described above, that are further specified in terms of their N-terminal and C-terminal positions. Also encompassed by the term “PTH fragment” as individual species are all PTH or PTHrP fragments, at least 6 amino acids in length, as described above, that may be particularly specified by a N-terminal and C-terminal position. That is, every combination of a N-terminal and C-terminal position that a fragment at least 6 contiguous amino acid residues in length could occupy, on any given amino acid sequence of a PTH or PTHrP, in certain embodiments the PTH of SEQ ID:NO51, is included in the present invention.

The term “PTH” also includes poly(amino acid) conjugates which have a sequence as described above, but having a backbone that comprises both amide and non-amide linkages, such as ester linkages, like for example depsipeptides. Depsipeptides are chains of amino acid residues in which the backbone comprises both amide (peptide) and ester bonds. Accordingly, the term “side chain” as used herein refers either to the moiety attached to the alpha-carbon of an amino acid moiety, if the amino acid moiety is connected through amine bonds such as in proteins and peptides, or to any carbon atom-comprising moiety attached to the backbone of a poly(amino acid) conjugate, such as for example in the case of depsipeptides. In certain embodiments the term “PTH” refers to sequences having a backbone formed through amide (peptide) bonds.

As the term PTH includes the above-described variants, analogs, orthologs, homologs, derivatives and fragments of PTH and PTHrP, all references to specific positions within a reference sequence also include the equivalent positions in variants, analogs, orthologs, homologs, derivatives and fragments of a PTH or PTHrP molecule or moiety, even if not specifically mentioned.

The term “peptide” as used herein refers to a chain of at least 2 and up to and including 50 amino acid monomer moieties, which may also be referred to as “amino acid residues”, linked by peptide (amide) linkages. The amino acid monomers may be selected from the group consisting of proteinogenic amino acids and non-proteinogenic amino acids and may be D- or L-amino acids. The term “peptide” also includes peptidomimetics, such as peptoids, beta-peptides, cyclic peptides and depsipeptides and covers such peptidomimetic chains with up to and including 50 monomer moieties.

As used herein, the term “protein” refers to a chain of more than 50 amino acid monomer moieties, which may also be referred to as “amino acid residues”, linked by peptide linkages, in which preferably no more than 12000 amino acid monomers are linked by peptide linkages, such as no more than 10000 amino acid monomer moieties, no more than 8000 amino acid monomer moieties, no more than 5000 amino acid monomer moieties or no more than 2000 amino acid monomer moieties.

As used herein the term “physiological conditions” refers to an aqueous buffer at pH 7.4, 37° C.

As used herein the term “pharmaceutical composition” refers to a composition containing one or more active ingredients, such as for example at least one PTH compound, and one or more excipients, as well as any product which results, directly or indirectly, from combination, complexation or aggregation of any two or more of the ingredients of the composition, or from dissociation of one or more of the ingredients, or from other types of reactions or interactions of one or more of the ingredients. Accordingly, a pharmaceutical composition for use of the present invention encompasses any composition made by admixing one or more PTH compound and a pharmaceutically acceptable excipient.

As used herein, the term “excipient” refers to a diluent, adjuvant, or vehicle with which the therapeutic, such as a drug or prodrug, is administered. Such pharmaceutical excipient can be sterile liquids, such as water and oils, including those of petroleum, animal, vegetable or synthetic origin, including but not limited to peanut oil, soybean oil, mineral oil, sesame oil and the like. Water is an example for an excipient when the pharmaceutical composition is administered orally. Saline and aqueous dextrose are examples of excipients when the pharmaceutical composition is administered intravenously. Saline solutions and aqueous dextrose and glycerol solutions are in certain embodiments employed as liquid excipients for injectable solutions. Suitable pharmaceutical excipients include starch, glucose, lactose, sucrose, mannitol, trehalose, gelatin, malt, rice, flour, chalk, silica gel, sodium stearate, glycerol monostearate, talc, sodium chloride, dried skim milk, glycerol, propylene, glycol, water, ethanol and the like. The pharmaceutical composition, if desired, can also contain minor amounts of wetting or emulsifying agents, pH buffering agents, like, for example, acetate, succinate, tris, carbonate, phosphate, HEPES (4-(2-hydroxyethyl)-1-piperazineethanesulfonic acid), MES (2-(N-morpholino)ethanesulfonic acid), or can contain detergents, like Tween, poloxamers, poloxamines, CHAPS, Igepal, or amino acids like, for example, glycine, lysine, or histidine. These pharmaceutical compositions can take the form of solutions, suspensions, emulsions, tablets, pills, capsules, powders, sustained-release formulations and the like. The pharmaceutical composition can be formulated as a suppository, with traditional binders and excipients such as triglycerides. Oral formulation can include standard excipients such as pharmaceutical grades of mannitol, lactose, starch, magnesium stearate, sodium saccharine, cellulose, magnesium carbonate, etc. Such compositions will contain a therapeutically effective amount of the drug or biologically active moiety, together with a suitable amount of excipient so as to provide the form for proper administration to the patient. The formulation should suit the mode of administration.

As used herein the term “liquid composition” refers to a mixture comprising a water-soluble PTH compound and one or more solvents, such as water.

The term “suspension composition” relates to a mixture comprising at least one PTH compound and one or more solvents, such as water.

As used herein, the term “dry composition” means that a pharmaceutical composition is provided in a dry form. Suitable methods for drying are spray-drying and lyophilization, i.e. freeze-drying. Such dry composition has a residual water content of a maximum of 10%, such as less than 5% or less than 2%, determined according to Karl Fischer. In certain embodiments such dry pharmaceutical composition is dried by lyophilization.

The term “drug” as used herein refers to a substance, such as PTH, used in the treatment, cure, prevention, or diagnosis of a disease or used to otherwise enhance physical or mental well-being. If a drug is conjugated to another moiety, the moiety of the resulting product that originated from the drug is referred to as “drug moiety”.

As used herein the term “prodrug” refers to a covalent conjugate in which a drug moiety is reversibly and covalently connected to a specialized protective group through a reversible linker moiety, also referred to as “reversible prodrug linker moiety” or “reversible linker moiety”, which comprises a reversible linkage with the biologically active moiety and wherein the specialized protective group alters or eliminates undesirable properties in the parent molecule. This also includes the enhancement of desirable properties in the drug and the suppression of undesirable properties. The specialized non-toxic protective group is referred to as “carrier”. A prodrug releases the reversibly and covalently bound drug moiety in the form of its corresponding drug. In other words, a prodrug is a conjugate comprising a drug moiety which is covalently and reversibly conjugated to a carrier moiety via a reversible linker moiety, which covalent and reversible conjugation of the carrier to the reversible linker moiety is either directly or through a spacer. Such conjugate releases the formerly conjugated drug moiety in the form of a free unmodified drug.

A “biodegradable linkage” or a “reversible linkage” is a linkage that is hydrolytically degradable, i.e. cleavable, in the absence of enzymes under physiological conditions (aqueous buffer at pH 7.4, 37° C.) with a half-life ranging from one hour to three months, in certain embodiments from one hour to two months, in certain embodiments from one hour to one month, in certain embodiments from one hour to three weeks, in certain embodiments from one hour to two weeks, in certain embodiments from 12 hours to two weeks, in certain embodiments from 12 hours to one week. Accordingly, a stable linkage is a linkage having a half-life under physiological conditions (aqueous buffer at pH 7.4, 37° C.) of more than three months.

As used herein, the terms “traceless prodrug linker” or “traceless linker” means a reversible prodrug linker, i.e. a linker moiety reversibly and covalently connecting the drug moiety with the carrier, which upon cleavage releases the drug in its free form. As used herein, the term “free form” of a drug means the drug in its unmodified, pharmacologically active form.

As used herein, the term “reagent” means a chemical compound which comprises at least one functional group for reaction with the functional group of another chemical compound or drug. It is understood that a drug comprising a functional group (such as a primary or secondary amine or hydroxyl functional group) is also a reagent.

As used herein, the term “moiety” means a part of a molecule, which lacks one or more atom(s) compared to the corresponding reagent. If, for example, a reagent of the formula “H—X—H” reacts with another reagent and becomes part of the reaction product, the corresponding moiety of the reaction product has the structure “H—X—” or “—X—”, whereas each “-” indicates attachment to another moiety. Accordingly, a drug moiety is released from a prodrug as a drug.

It is understood that if the chemical structure of a group of atoms is provided which group of atoms is attached to at least one other moiety or is interrupting a moiety, said chemical structure may be attached to the at least one further or interrupted moiety in either orientation, unless explicitly stated otherwise. For example, a moiety “—C(O)N(R¹)—” may be attached to two moieties or interrupting a moiety either as “—C(O)N(R¹)—” or as “—N(R¹)C(O)—”. Similarly, a moiety

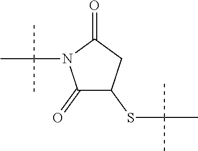

may be attached to two moieties or can interrupt a moiety either as

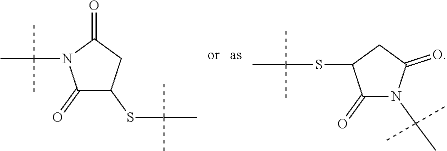

As used herein, the term “functional group” means a group of atoms which can react with other groups of atoms. Functional groups include but are not limited to the following groups: carboxylic acid (—(C═O)OH), primary or secondary amine (—NH₂, —NH—), maleimide, thiol (—SH), sulfonic acid (—(O=S=O)OH), carbonate, carbamate (—O(C═O)N<), hydroxyl (—OH), aldehyde (—(C═O)H), ketone (—(C═O)—), hydrazine (>N—N<), isocyanate, isothiocyanate, phosphoric acid (—O(P═O)OHOH), phosphonic acid (—O(P═O)OHH), haloacetyl, alkyl halide, acryloyl, aryl fluoride, hydroxylamine, disulfide, sulfonamides, sulfuric acid, vinyl sulfone, vinyl ketone, diazoalkane, oxirane, and aziridine.

In case the PTH compound for use of the present invention comprises one or more acidic or basic groups, the invention also comprises their corresponding pharmaceutically or toxicologically acceptable salts, in particular their pharmaceutically utilizable salts. Thus, the PTH compound for use of the present invention comprising acidic groups may be used according to the invention, for example, as alkali metal salts, alkaline earth metal salts or as ammonium salts. More precise examples of such salts include sodium salts, potassium salts, calcium salts, magnesium salts or salts with ammonia or organic amines such as, for example, ethylamine, ethanolamine, triethanolamine or amino acids. A PTH compound for use of the present invention comprising one or more basic groups, i.e. groups which can be protonated, may be present and may be used according to the invention in the form of their addition salts with inorganic or organic acids. Examples for suitable acids include hydrogen chloride, hydrogen bromide, phosphoric acid, sulfuric acid, nitric acid, methanesulfonic acid, p-toluenesulfonic acid, naphthalenedisulfonic acids, oxalic acid, acetic acid, tartaric acid, lactic acid, salicylic acid, benzoic acid, formic acid, propionic acid, pivalic acid, diethylacetic acid, malonic acid, succinic acid, pimelic acid, fumaric acid, maleic acid, malic acid, sulfaminic acid, phenylpropionic acid, gluconic acid, ascorbic acid, isonicotinic acid, citric acid, adipic acid, and other acids known to the person skilled in the art. For the person skilled in the art further methods are known for converting the basic group into a cation like the alkylation of an amine group resulting in a positively-charge ammonium group and an appropriate counterion of the salt. If the PTH compound for use of the present invention simultaneously comprises acidic and basic groups, the invention also includes, in addition to the salt forms mentioned above, inner salts or betaines (zwitterions). The respective salts may be obtained by customary methods which are known to the person skilled in the art like, for example by contacting these compounds with an organic or inorganic acid or base in a solvent or dispersant, or by anion exchange or cation exchange with other salts. The present invention also includes all salts of the compounds for use of the present invention which, owing to low physiological compatibility, are not directly suitable for use in pharmaceuticals but which can be used, for example, as intermediates for chemical reactions or for the preparation of pharmaceutically acceptable salts.

The term “pharmaceutically acceptable” means a substance that does not cause harm when administered to a patient and in certain embodiments means approved by a regulatory agency, such as the EMA (Europe) and/or the FDA (US) and/or any other national regulatory agency for use in animals, in particular for use in humans.

As used herein the term “about” in combination with a numerical value is used to indicate a range ranging from and including the numerical value plus and minus no more than 10% of said numerical value, in certain embodiments no more than 8% of said numerical value, in certain embodiments no more than 5% of said numerical value and in certain embodiments no more than 2% of said numerical value. For example, the phrase “about 200” is used to mean a range ranging from and including 200+/−10%, i.e. ranging from and including 180 to 220; in certain embodiments 200+/−8%, i.e. ranging from and including 184 to 216; in certain embodiments ranging from and including 200+/−5%, i.e. ranging from and including 190 to 210; and in certain embodiments 200+/−2%, i.e. ranging from and including 196 to 204. It is understood that a percentage given as “about 20%” does not mean “20%+/−10%”, i.e. ranging from and including 10 to 30%, but “about 20%” means ranging from and including 18 to 22%, i.e. plus and minus 10% of the numerical value which is 20.

As used herein, the term “polymer” means a molecule comprising repeating structural units, i.e. the monomers, connected by chemical bonds in a linear, circular, branched, crosslinked or dendrimeric way or a combination thereof, which may be of synthetic or biological origin or a combination of both. It is understood that a polymer may also comprise one or more other chemical groups and/or moieties, such as, for example, one or more functional groups. In certain embodiments a soluble polymer has a molecular weight of at least 0.5 kDa, e.g. a molecular weight of at least 1 kDa, a molecular weight of at least 2 kDa, a molecular weight of at least 3 kDa or a molecular weight of at least 5 kDa. If the polymer is soluble, it in certain embodiments has a molecular weight of at most 1000 kDa, such as at most 750 kDa, such as at most 500 kDa, such as at most 300 kDa, such as at most 200 kDa, such as at most 100 kDa. It is understood that for water-insoluble polymers, such as hydrogels, no meaningful molecular weight ranges can be provided. It is understood that also a peptide or protein is a polymer in which the amino acids are the repeating structural units, even though the side chains of each amino acid may be different.

As used herein, the term “polymeric” means a reagent or a moiety comprising one or more polymers or polymer moieties. A polymeric reagent or moiety may optionally also comprise one or more other moiety/moieties, which are in certain embodiments selected from the group consisting of:
C₁₋₅₀ alkyl, C₂₋₅₀ alkenyl, C₂₋₅₀ alkynyl, C₃₋₁₀ cycloalkyl, 3- to 10-membered heterocyclyl, 8- to 11-membered heterobicyclyl, phenyl, naphthyl, indenyl, indanyl, and tetralinyl; and
linkages selected from the group comprising

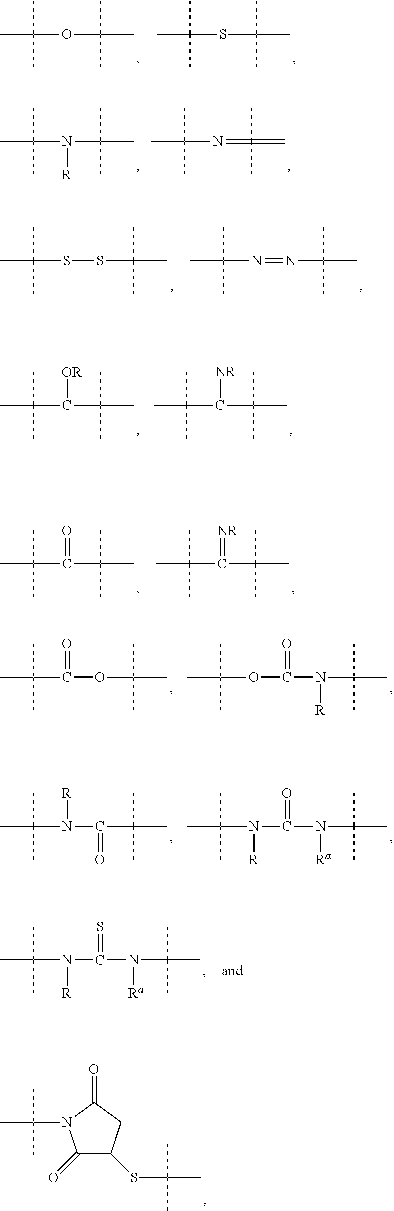

wherein
dashed lines indicate attachment to the remainder of the moiety or reagent, and —R and —R^{a} are independently of each other selected from the group consisting of —H, methyl, ethyl, n-propyl, isopropyl, n-butyl, isobutyl, sec-butyl, tert-butyl, n-pentyl, 2-methylbutyl, 2,2-dimethylpropyl, n-hexyl, 2-methylpentyl, 3-methylpentyl, 2,2-dimethylbutyl, 2,3-dimethylbutyl and 3,3-dimethylpropyl.

The person skilled in the art understands that the polymerization products obtained from a polymerization reaction do not all have the same molecular weight, but rather exhibit a molecular weight distribution. Consequently, the molecular weight ranges, molecular weights, ranges of numbers of monomers in a polymer and numbers of monomers in a polymer as used herein, refer to the number average molecular weight and number average of monomers, i.e. to the arithmetic mean of the molecular weight of the polymer or polymeric moiety and the arithmetic mean of the number of monomers of the polymer or polymeric moiety.

Accordingly, in a polymeric moiety comprising “x” monomer units any integer given for “x” therefore corresponds to the arithmetic mean number of monomers. Any range of integers given for “x” provides the range of integers in which the arithmetic mean numbers of monomers lies. An integer for “x” given as “about x” means that the arithmetic mean numbers of monomers lies in a range of integers of x+/−10%, in certain embodiments x+/−8%, in certain embodiments x+/−5% and in certain embodiments x+/−2%.

As used herein, the term “number average molecular weight” means the ordinary arithmetic mean of the molecular weights of the individual polymers.

As used herein the term “water-soluble” with reference to a carrier means that when such carrier is part of the PTH compound for use of the present invention at least 1 g of the PTH compound comprising such water-soluble carrier may be dissolved in one liter of water at 20° C. to form a homogeneous solution. Accordingly, the term “water-insoluble” with reference to a carrier means that when such carrier is part of a PTH compound for use of the present invention less than 1 g of the PTH compound comprising such water-insoluble carrier can be dissolved in one liter of water at 20° C. to form a homogeneous solution.

As used herein the term “water-soluble” with reference to the PTH compound means that at least 1 g of the PTH compound may be dissolved in one liter of water at 20° C. to form a homogeneous solution. Accordingly, the term “water-insoluble” with reference to the PTH compound means that less than 1 g of the PTH compound may be dissolved in one liter of water at 20° C. to form a homogeneous solution.

As used herein, the term “PEG-based” in relation to a moiety or reagent means that said moiety or reagent comprises PEG. In certain embodiments a PEG-based moiety or reagent comprises at least 10% (w/w) PEG, such as at least 20% (w/w) PEG, such as at least 30% (w/w) PEG, such as at least 40% (w/w) PEG, such as at least 50% (w/w), such as at least 60 (w/w) PEG, such as at least 70% (w/w) PEG, such as at least 80% (w/w) PEG, such as at least 90% (w/w) PEG, such as at least 95%. The remaining weight percentage of the PEG-based moiety or reagent are other moieties that in certain embodiments are selected from the following moieties and linkages:
C₁₋₅₀ alkyl, C₂₋₅₀ alkenyl, C₂₋₅₀ alkynyl, C₃₋₁₀ cycloalkyl, 3- to 10-membered heterocyclyl, 8- to 11-membered heterobicyclyl, phenyl, naphthyl, indenyl, indanyl, and tetralinyl; and
linkages selected from the group comprising

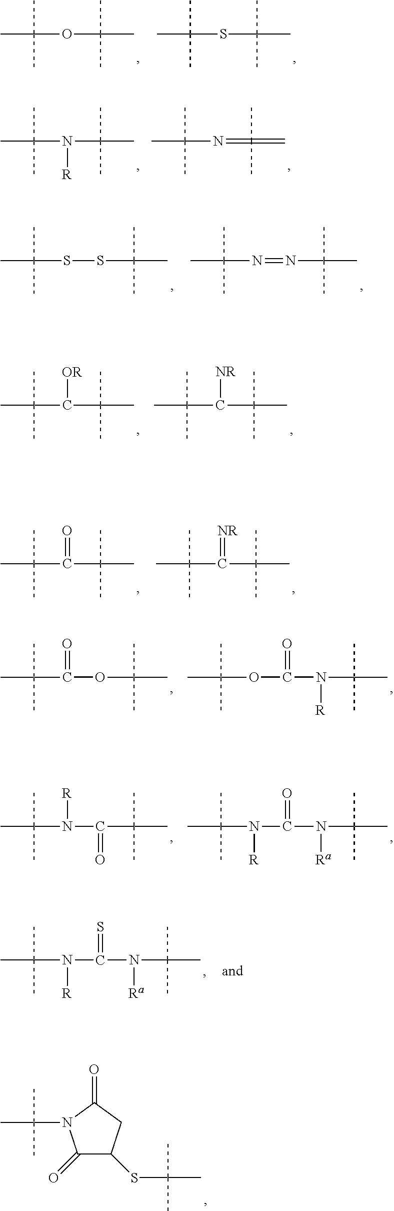

wherein
dashed lines indicate attachment to the remainder of the moiety or reagent, and —R and —R^{a} are independently of each other selected from the group consisting of —H, methyl, ethyl, n-propyl, isopropyl, n-butyl, isobutyl, sec-butyl, tert-butyl, n-pentyl, 2-methylbutyl, 2,2-dimethylpropyl, n-hexyl, 2-methylpentyl, 3-methylpentyl, 2,2-dimethylbutyl, 2,3-dimethylbutyl and 3,3-dimethylpropyl.

As used herein, the term “PEG-based comprising at least X % PEG” in relation to a moiety or reagent means that said moiety or reagent comprises at least X % (w/w) ethylene glycol units (—CH₂CH₂O—), wherein the ethylene glycol units may be arranged blockwise, alternating or may be randomly distributed within the moiety or reagent and in certain embodiments all ethylene glycol units of said moiety or reagent are present in one block; the remaining weight percentage of the PEG-based moiety or reagent are other moieties that in certain embodiments are selected from the following moieties and linkages:
C₁₋₅₀ alkyl, C₂₋₅₀ alkenyl, C₂₋₅₀ alkynyl, C₃₋₁₀ cycloalkyl, 3- to 10-membered heterocyclyl, 8- to 11-membered heterobicyclyl, phenyl, naphthyl, indenyl, indanyl, and tetralinyl; and
linkages selected from the group comprising

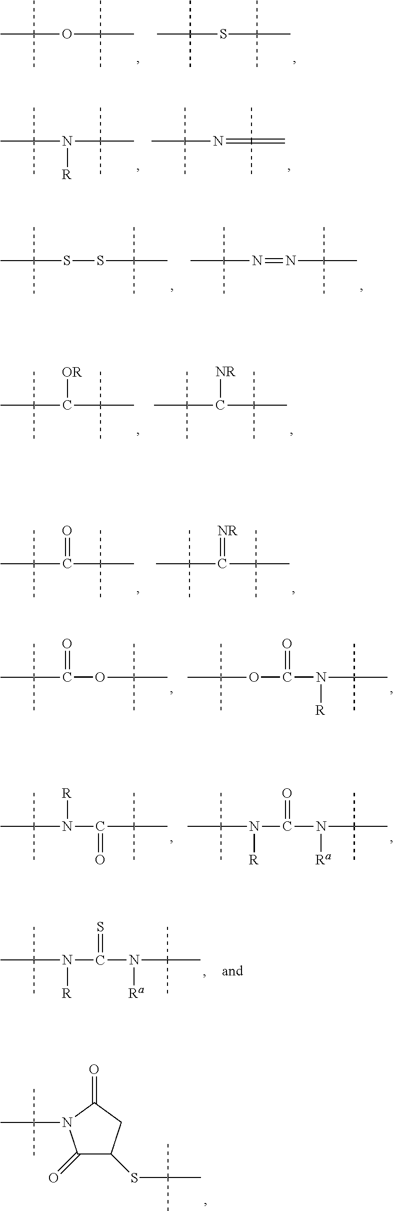

wherein
dashed lines indicate attachment to the remainder of the moiety or reagent, and —R and —R^{a} are independently of each other selected from the group consisting of —H, methyl, ethyl, n-propyl, isopropyl, n-butyl, isobutyl, sec-butyl, tert-butyl, n-pentyl, 2-methylbutyl, 2,2-dimethylpropyl, n-hexyl, 2-methylpentyl, 3-methylpentyl, 2,2-dimethylbutyl, 2,3-dimethylbutyl and 3,3-dimethylpropyl.

The term “hyaluronic acid-based comprising at least X % hyaluronic acid” is used accordingly.

The term “substituted” as used herein means that one or more —H atom(s) of a molecule or moiety are replaced by a different atom or a group of atoms, which are referred to as “substituent”.

In certain embodiments the one or more further optional substituents are independently of each other selected from the group consisting of halogen, —CN, —COOR^{x1}, —OR^{x1}, —C(O)R^{x1}, —C(O)N(R^{x1}R^{x1a}), —S(O)₂N(R^{x1}R^{x1a}), —S(O)N(R^{x1}R^{x1a}), —S(O)₂R^{x1}, —S(O)R^{x1}, —N(R^{x1})S(O)₂N(Rₓ₁ₐR^{x1b}), —SR^{x1}, —N(R^{x1}R^{x1a}), —NO₂, —OC(O)R^{x1}, —N(R^{x1})C(O)R^{x1a}, —N(R^{x1})S(O)₂R^{x1a}, —N(R^{x1})S(O)R^{x1a}, —N(R^{x1})C(O)OR^{x1a}, —N(R^{x1})C(O)N(R^{x1a}R^{x1b}), —OC(O)N(R^{x1}R^{x1a}), -T⁰, C₁₋₅₀ alkyl, C₂₋₅₀ alkenyl, and C₂₋₅₀ alkynyl; wherein -T⁰, C₁₋₅₀ alkyl, C₂₋₅₀ alkenyl, and C₂₋₅₀ alkynyl are optionally substituted with one or more —R^{x2}, which are the same or different and wherein C₁₋₅₀ alkyl, C₂₋₅₀ alkenyl, and C₂₋₅₀ alkynyl are optionally interrupted by one or more groups selected from the group consisting of -T⁰-, —C(O)O—, —O—, —C(O)—, —C(O)N(R^{x3})—, —S(O)₂N(R^{x3})—, —S(O)N(R^{x3})—, —S(O)₂—, —S(O)—, —N(R^{x3})S(O)₂N(R^{x3a})—, —S—, —N(R^{x3})—, —OC(OR^{x3})(R^{x3a})—, —N(R^{x3})C(O)N(R^{x3a})—, and —OC(O)N(R^{x3})—; —R^{x1}, —R^{x1a}, —R^{x1b} are independently of each other selected from the group consisting of —H, -T⁰, C₁₋₅₀ alkyl, C₂₋₅₀ alkenyl, and C₂₋₅₀ alkynyl; wherein -T⁰, C₁₋₅₀ alkyl, C₂₋₅₀ alkenyl, and C₂₋₅₀ alkynyl are optionally substituted with one or more —R^{x2}, which are the same or different and wherein C₁₋₅₀ alkyl, C₂₋₅₀ alkenyl, and C₂₋₅₀ alkynyl are optionally interrupted by one or more groups selected from the group consisting of -T⁰-, —C(O)O—, —O—, —C(O)—, —C(O)N(R^{x3})—, —S(O)₂N(R^{x3})—, —S(O)N(R^{x3})—; —S(O)₂—, —S(O)—, —N(R^{x3})S(O)₂N(R^{x3a})—, —S—, —N(R^{x3})—, —OC(OR^{x3})(R^{x3a})—, —N(R^{x3})C(O)N(R^{x3a})—, and —OC(O)N(R^{x3})—;

each T⁰ is independently selected from the group consisting of phenyl, naphthyl, indenyl, indanyl, tetralinyl, C₃₋₁₀ cycloalkyl, 3- to 10-membered heterocyclyl, and 8- to 11-membered heterobicyclyl; wherein each T⁰ is independently optionally substituted with one or more —R^{x2} which are the same or different;

each —R^{x2} is independently selected from the group consisting of halogen, —CN, oxo (═O), —COOR^{x4}, —OR^{x4}, —C(O)R^{x4}, —C(O)N(R^{x4}R^{x4a}), —S(O)₂N(R^{x4}R^{x4a}), —S(O)N(R^{x4}R^{x4a}), —S(O)₂R^{x4}, —S(O)R^{x4}, —N(R^{x4})S(O)₂N(R^{x4a}R^{x4b}), —SR^{x4}, —N(R^{x4}R^{x4a}), —NO₂, —OC(O)R^{x4}, —N(R^{x4})C(O)R^{x4a}, —N(R^{x4})S(O)₂R^{x4a}, —N(R^{x4})S(O)R^{x4a}, —N(R^{x4})C(O)OR^{x4a}, —N(R^{x4})C(O)N(R^{x4a}R^{x4b}), —OC(O)N(R^{x4}R^{x4a}), and C₁₋₆ alkyl; wherein C₁₋₆ alkyl is optionally substituted with one or more halogen, which are the same or different;

each —R^{x3}, —R^{x3a}, —R^{x4}, —R^{x4a}, —R^{x4b} is independently selected from the group consisting of —H and C₁₋₆ alkyl; wherein C₁₋₆ alkyl is optionally substituted with one or more halogen, which are the same or different.

In certain embodiments the one or more further optional substituents are independently of each other selected from the group consisting of halogen, —CN, —COOR^{x1}, —OR^{x1}, —C(O)R^{x1}, —C(O)N(R^{x1}R^{x1a}), —S(O)₂N(R^{x1}R^{x1a}), —S(O)N(R^{x1}R^{x1a}), —S(O)₂R^{x1}, —S(O)R^{x1}, —N(R^{x1})S(O)₂N(R^{x1a}R^{x1b}), —SR^{x1}, —N(R^{x1}R^{x1a}), —NO₂, —OC(O)R^{x1}, —N(R^{x1})C(O)R^{x1a}, —N(R^{x1})S(O)₂R^{x1a}, —N(R^{x1})S(O)R^{x1a}, —N(R^{x1})C(O)OR^{x1a}, —N(R^{x1})C(O)N(R^{x1a}R^{x1b}), —OC(O)N(R^{x1}R^{x1a}), -T⁰, C₁₋₁₀ alkyl, C₂₋₁₀ alkenyl, and C₂₋₁₀ alkynyl; wherein -T⁰, C₁₋₁₀ alkyl, C₂₋₁₀ alkenyl, and C₂₋₁₀ alkynyl are optionally substituted with one or more —R^{x2}, which are the same or different and wherein C₁₋₁₀ alkyl, C₂₋₁₀ alkenyl, and C₂₋₁₀ alkynyl are optionally interrupted by one or more groups selected from the group consisting of -T⁰-, —C(O)O—, —O—, —C(O)—, —C(O)N(R^{x3})—, —S(O)₂N(R^{x3})—, —S(O)N(R^{x3})—, —S(O)₂—, —S(O)—, —N(R^{x3})S(O)₂N(R^{x3a})—, —S—, —N(R^{x3})—, —OC(OR^{x3})(R^{x3a})—, —N(R^{x3})C(O)N(R^{x3a})—, and —OC(O)N(R^{x3})—;

each —R^{x1}, —R^{x1a}, —R^{x1b}, —R^{x3}, —R^{x3a} is independently selected from the group consisting of —H, halogen, C₁₋₆ alkyl, C₂₋₆ alkenyl, and C₂₋₆ alkynyl;

each T⁰ is independently selected from the group consisting of phenyl, naphthyl, indenyl, indanyl, tetralinyl, C₃₋₁₀ cycloalkyl, 3- to 10-membered heterocyclyl, and 8- to 11-membered heterobicyclyl; wherein each T⁰ is independently optionally substituted with one or more —R^{x2} which are the same or different;

each —R^{x2} is independently selected from the group consisting of halogen, —CN, oxo (═O), —COOR^{x4}, —OR^{x4}, —C(O)R^{x4}, —C(O)N(R^{x4}R^{x4a}), —S(O)₂N(R^{x4}R^{x4a}), —S(O)N(R^{x4}R^{x4a}), —S(O)₂R^{x4}, —S(O)R^{x4}, —N(R^{x4})S(O)₂N(R^{x4a}R^{x4b}), —SR^{x4}, —N(R^{x4}R^{x4a}), —NO₂, —OC(O)R^{x4}, —N(R^{x4})C(O)R^{x4a}, —N(R^{x4})S(O)₂R^{x4a}, —N(R^{x4})S(O)R^{x4a}, —N(R^{x4})C(O)OR^{x4a}, —N(R^{x4})C(O)N(R^{x4a}R^{x4b}), —OC(O)N(R^{x4}R^{x4a}), and C₁₋₆ alkyl; wherein C₁₋₆ alkyl is optionally substituted with one or more halogen, which are the same or different;

each —R^{x4}, —R^{x4a}, —R^{x4b} is independently selected from the group consisting of —H, halogen, C₁₋₆ alkyl, C₂₋₆ alkenyl, and C₂₋₆ alkynyl;

In certain embodiments the one or more further optional substituents are independently of each other selected from the group consisting of halogen, —CN, —COOR^{x1}, —OR^{x1}, —C(O)R^{x1}, —C(O)N(R^{x1}R^{x1a}), —S(O)₂N(R^{x1}R^{x1a}), —S(O)N(R^{x1}R^{x1a}), —S(O)₂R^{x1}, —S(O)R^{x1}, —N(R^{x1})S(O)₂N(R^{x1a}R^{x1b}), —SR^{x1}, —N(R^{x1}R^{x1a}), —NO₂, —OC(O)R^{x1}, —N(R^{x1})C(O)R^{x1a}, —N(R^{x1})S(O)₂R^{x1a}, —N(R^{x1})S(O)R^{x1a}, —N(R^{x1})C(O)OR^{x1a}, —N(R^{x1})C(O)N(R^{x1a}R^{x1b}), —OC(O)N(R^{x1}R^{x1a}), -T⁰, C₁₋₆ alkyl, C₂₋₆ alkenyl, and C₂₋₆ alkynyl; wherein -T⁰, C₁₋₆ alkyl, C₂₋₆ alkenyl, and C₂₋₆ alkynyl are optionally substituted with one or more —R^{x2}, which are the same or different and wherein C₁₋₆ alkyl, C₂₋₆ alkenyl, and C₂₋₆ alkynyl are optionally interrupted by one or more groups selected from the group consisting of -T⁰-, —C(O)O—, —O—, —C(O)—, —C(O)N(R^{x3})—, —S(O)₂N(R^{x3})—, —S(O)N(R^{x3})—, —S(O)₂—, —S(O)—, —N(R^{x3})S(O)₂N(R^{x3a})—, —S—, —N(R^{x3})—, —OC(OR^{x3})(R^{x3a})—, —N(R^{x3})C(O)N(R^{x3a})—, and —OC(O)N(R^{x3})—;

each —R^{x1}, —R^{x1a}, —R^{x1b}, —R^{x2}, —R^{x3}, —R^{x3a} is independently selected from the group consisting of —H, halogen, C₁₋₆ alkyl, C₂₋₆ alkenyl, and C₂₋₆ alkynyl;

each T⁰ is independently selected from the group consisting of phenyl, naphthyl, indenyl, indanyl, tetralinyl, C₃₋₁₀ cycloalkyl, 3- to 10-membered heterocyclyl, and 8- to 11-membered heterobicyclyl; wherein each T⁰ is independently optionally substituted with one or more —R^{x2}, which are the same or different.

In certain embodiments a maximum of 6 —H atoms of an optionally substituted molecule are independently replaced by a substituent, e.g. 5 —H atoms are independently replaced by a substituent, 4 —H atoms are independently replaced by a substituent, 3 —H atoms are independently replaced by a substituent, 2 —H atoms are independently replaced by a substituent, or 1 —H atom is replaced by a substituent.

The term “interrupted” means that a moiety is inserted between two carbon atoms or—if the insertion is at one of the moiety's ends—between a carbon or heteroatom and a hydrogen atom, in certain embodiments between a carbon and a hydrogen atom.

As used herein, the term “C₁₋₄ alkyl” alone or in combination means a straight-chain or branched alkyl moiety having 1 to 4 carbon atoms. If present at the end of a molecule, examples of straight-chain or branched C₁₋₄ alkyl are methyl, ethyl, n-propyl, isopropyl, n-butyl, isobutyl, sec-butyl and tert-butyl. When two moieties of a molecule are linked by the C₁₋₄ alkyl, then examples for such C₁₋₄ alkyl groups are —CH₂—, —CH₂—CH₂—, —CH(CH₃)—, —CH₂—CH₂—CH₂—, —CH(C₂H₅)—, —C(CH₃)₂—. Each hydrogen of a C₁₋₄ alkyl carbon may optionally be replaced by a substituent as defined above. Optionally, a C₁₋₄ alkyl may be interrupted by one or more moieties as defined below.

As used herein, the term “C₁₋₆ alkyl” alone or in combination means a straight-chain or branched alkyl moiety having 1 to 6 carbon atoms. If present at the end of a molecule, examples of straight-chain and branched C₁₋₆ alkyl groups are methyl, ethyl, n-propyl, isopropyl, n-butyl, isobutyl, sec-butyl, tert-butyl, n-pentyl, 2-methylbutyl, 2,2-dimethylpropyl, n-hexyl, 2-methylpentyl, 3-methylpentyl, 2,2-dimethylbutyl, 2,3-dimethylbutyl and 3,3-dimethylpropyl. When two moieties of a molecule are linked by the C₁₋₆ alkyl group, then examples for such C₁₋₆ alkyl groups are —CH₂—, —CH₂—CH₂—, —CH(CH₃)—, —CH₂—CH₂—CH₂—, —CH(C₂H₅)— and —C(CH₃)₂—. Each hydrogen atom of a C₁₋₆ carbon may optionally be replaced by a substituent as defined above. Optionally, a C₁₋₆ alkyl may be interrupted by one or more moieties as defined below.

Accordingly, “C₁₋₁₀ alkyl”, “C₁₋₂₀ alkyl” or “C₁₋₅₀ alkyl” means an alkyl chain having 1 to 10, 1 to 20 or 1 to 50 carbon atoms, respectively, wherein each hydrogen atom of the C₁₋₁₀, C₁₋₂₀ or C₁₋₅₀ carbon may optionally be replaced by a substituent as defined above. Optionally, a C₁₋₁₀ or C₁₋₅₀ alkyl may be interrupted by one or more moieties as defined below.

As used herein, the term “C₂₋₆ alkenyl” alone or in combination means a straight-chain or branched hydrocarbon moiety comprising at least one carbon-carbon double bond having 2 to 6 carbon atoms. If present at the end of a molecule, examples are —CH═CH₂, —CH═CH—CH₃, —CH₂—CH═CH₂, —CH═CHCH₂—CH₃ and —CH═CH—CH═CH₂. When two moieties of a molecule are linked by the C₂₋₆ alkenyl group, then an example for such C₂₋₆ alkenyl is —CH═CH—. Each hydrogen atom of a C₂₋₆ alkenyl moiety may optionally be replaced by a substituent as defined above. Optionally, a C₂₋₆ alkenyl may be interrupted by one or more moieties as defined below.

Accordingly, the term “C₂₋₁₀ alkenyl”, “C₂₋₂₀ alkenyl” or “C₂₋₅₀ alkenyl” alone or in combination means a straight-chain or branched hydrocarbon moiety comprising at least one carbon-carbon double bond having 2 to 10, 2 to 20 or 2 to 50 carbon atoms. Each hydrogen atom of a C₂₋₁₀ alkenyl, C₂₋₂₀ alkenyl or C₂₋₅₀ alkenyl group may optionally be replaced by a substituent as defined above. Optionally, a C₂₋₁₀ alkenyl, C₂₋₂₀ alkenyl or C₂₋₅₀ alkenyl may be interrupted by one or more moieties as defined below.

As used herein, the term “C₂₋₆ alkynyl” alone or in combination means straight-chain or branched hydrocarbon moiety comprising at least one carbon-carbon triple bond having 2 to 6 carbon atoms. If present at the end of a molecule, examples are —C≡CH, —CH₂—C≡CH, CH₂—CH₂—C≡CH and CH₂—C≡C—CH₃. When two moieties of a molecule are linked by the alkynyl group, then an example is —C≡C—. Each hydrogen atom of a C₂₋₆ alkynyl group may optionally be replaced by a substituent as defined above. Optionally, one or more double bond(s) may occur. Optionally, a C₂₋₆ alkynyl may be interrupted by one or more moieties as defined below.

Accordingly, as used herein, the term “C₂₋₁₀ alkynyl”, “C₂₋₂₀ alkynyl” and “C₂₋₅₀ alkynyl” alone or in combination means a straight-chain or branched hydrocarbon moiety comprising at least one carbon-carbon triple bond having 2 to 10, 2 to 20 or 2 to 50 carbon atoms, respectively. Each hydrogen atom of a C₂₋₁₀ alkynyl, C₂₋₂₀ alkynyl or C₂₋₅₀ alkynyl group may optionally be replaced by a substituent as defined above. Optionally, one or more double bond(s) may occur. Optionally, a C₂₋₁₀ alkynyl, C₂₋₂₀ alkynyl or C₂₋₅₀ alkynyl may be interrupted by one or more moieties as defined below.

As mentioned above, a Cia alkyl, C₁₋₆ alkyl, C₁₋₁₀ alkyl, C₁₋₂₀ alkyl, C₁₋₅₀ alkyl, C₂₋₆ alkenyl, C₂₋₁₀ alkenyl, C₂₋₂₀ alkenyl, C₂₋₅₀ alkenyl, C₂₋₆ alkynyl, C₂₋₁₀ alkynyl, C₂₋₂₀ alkenyl or C₂₋₅₀ alkynyl may optionally be interrupted by one or more moieties which in certain embodiments are selected from the group consisting of

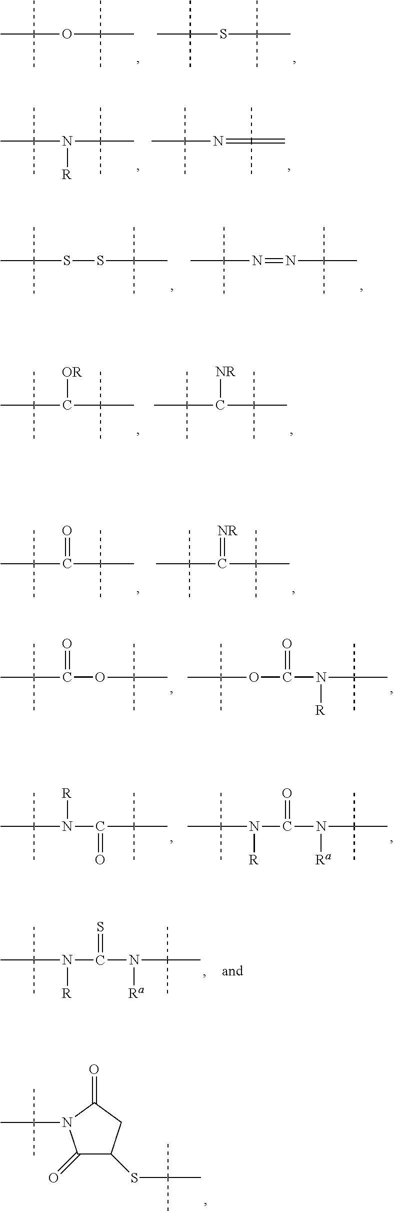

wherein
dashed lines indicate attachment to the remainder of the moiety or reagent; and —R and —R^{a} are independently of each other selected from the group consisting of —H, methyl, ethyl, n-propyl, isopropyl, n-butyl, isobutyl, sec-butyl, tert-butyl, n-pentyl, 2-methylbutyl, 2,2-dimethylpropyl, n-hexyl, 2-methylpentyl, 3-methylpentyl, 2,2-dimethylbutyl, 2,3-dimethylbutyl and 3,3-dimethylpropyl.

As used herein, the term “C₃₋₁₀ cycloalkyl” means a cyclic alkyl chain having 3 to 10 carbon atoms, which may be saturated or unsaturated, e.g. cyclopropyl, cyclobutyl, cyclopentyl, cyclohexyl, cyclohexenyl, cycloheptyl, cyclooctyl, cyclononyl or cyclodecyl. Each hydrogen atom of a C₃₋₁₀ cycloalkyl carbon may be replaced by a substituent as defined above. The term “C₃₋₁₀ cycloalkyl” also includes bridged bicycles like norbornane or norbornene.

The term “8- to 30-membered carbopolycyclyl” or “8- to 30-membered carbopolycycle” means a cyclic moiety of two or more rings with 8 to 30 ring atoms, where two neighboring rings share at least one ring atom and that may contain up to the maximum number of double bonds (aromatic or non-aromatic ring which is fully, partially or un-saturated). In certain embodiments a 8- to 30-membered carbopolycyclyl means a cyclic moiety of two, three, four or five rings, in certain embodiments of two, three or four rings.

As used herein, the term “3- to 10-membered heterocyclyl” or “3- to 10-membered heterocycle” means a ring with 3, 4, 5, 6, 7, 8, 9 or 10 ring atoms that may contain up to the maximum number of double bonds (aromatic or non-aromatic ring which is fully, partially or un-saturated) wherein at least one ring atom up to 4 ring atoms are replaced by a heteroatom selected from the group consisting of sulfur (including —S(O)—, —S(O)₂—), oxygen and nitrogen (including ═N(O)—) and wherein the ring is linked to the rest of the molecule via a carbon or nitrogen atom. Examples for 3- to 10-membered heterocycles include but are not limited to aziridine, oxirane, thiirane, azirine, oxirene, thiirene, azetidine, oxetane, thietane, furan, thiophene, pyrrole, pyrroline, imidazole, imidazoline, pyrazole, pyrazoline, oxazole, oxazoline, isoxazole, isoxazoline, thiazole, thiazoline, isothiazole, isothiazoline, thiadiazole, thiadiazoline, tetrahydrofuran, tetrahydrothiophene, pyrrolidine, imidazolidine, pyrazolidine, oxazolidine, isoxazolidine, thiazolidine, isothiazolidine, thiadiazolidine, sulfolane, pyran, dihydropyran, tetrahydropyran, imidazolidine, pyridine, pyridazine, pyrazine, pyrimidine, piperazine, piperidine, morpholine, tetrazole, triazole, triazolidine, tetrazolidine, diazepane, azepine and homopiperazine. Each hydrogen atom of a 3- to 10-membered heterocyclyl or 3- to 10-membered heterocyclic group may be replaced by a substituent as defined below.

As used herein, the term “8- to 11-membered heterobicyclyl” or “8- to 11-membered heterobicycle” means a heterocyclic moiety of two rings with 8 to 11 ring atoms, where at least one ring atom is shared by both rings and that may contain up to the maximum number of double bonds (aromatic or non-aromatic ring which is fully, partially or un-saturated) wherein at least one ring atom up to 6 ring atoms are replaced by a heteroatom selected from the group consisting of sulfur (including —S(O)—, —S(O)₂—), oxygen and nitrogen (including ═N(O)—) and wherein the ring is linked to the rest of the molecule via a carbon or nitrogen atom. Examples for an 8- to 11-membered heterobicycle are indole, indoline, benzofuran, benzothiophene, benzoxazole, benzisoxazole, benzothiazole, benzisothiazole, benzimidazole, benzimidazoline, quinoline, quinazoline, dihydroquinazoline, quinoline, dihydroquinoline, tetrahydroquinoline, decahydroquinoline, isoquinoline, decahydroisoquinoline, tetrahydroisoquinoline, dihydroisoquinoline, benzazepine, purine and pteridine. The term 8- to 11-membered heterobicycle also includes spiro structures of two rings like 1,4-dioxa-8-azaspiro[4.5]decane or bridged heterocycles like 8-aza-bicyclo[3.2.1]octane. Each hydrogen atom of an 8- to 11-membered heterobicyclyl or 8- to 11-membered heterobicycle carbon may be replaced by a substituent as defined below.

Similarly, the term “8- to 30-membered heteropolycyclyl” or “8- to 30-membered heteropolycycle” means a heterocyclic moiety of more than two rings with 8 to 30 ring atoms, in certain embodiments of three, four or five rings, where two neighboring rings share at least one ring atom and that may contain up to the maximum number of double bonds (aromatic or non-aromatic ring which is fully, partially or unsaturated), wherein at least one ring atom up to 10 ring atoms are replaced by a heteroatom selected from the group of sulfur (including —S(O)—, —S(O)₂—), oxygen and nitrogen (including ═N(O)—) and wherein the ring is linked to the rest of a molecule via a carbon or nitrogen atom.

It is understood that the phrase “the pair R^{x}/R^{y} is joined together with the atom to which they are attached to form a C₃₋₁₀ cycloalkyl or a 3- to 10-membered heterocyclyl” in relation with a moiety of the structure

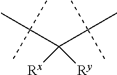

means that R^{x} and R^{y} form the following structure:

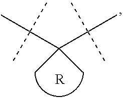

wherein R is C₃₋₁₀ cycloalkyl or 3- to 10-membered heterocyclyl.

It is also understood that the phrase “the pair R^{x}/R^{y} is joint together with the atoms to which they are attached to form a ring A” in relation with a moiety of the structure

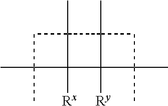

means that R^{x} and R^{y} form the following structure:

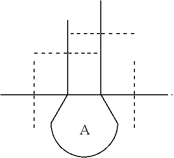

As used herein, “halogen” means fluoro, chloro, bromo or iodo. In certain embodiments halogen is fluoro or chloro.

In general, the term “comprise” or “comprising” also encompasses “consist of” or “consisting of”.

In certain embodiments the patient is titrated off standard of care within four weeks from the time of the first dose of PTH compound is administered. In certain embodiments the patient is titrated off standard of care within three weeks from the time the first dose of the PTH compound is administered. In certain embodiments the patient is titrated off standard of care within two weeks from the time the first dose of the PTH compound is administered. In certain embodiments the patient is titrated off standard of care within two weeks from the time the first dose of the PTH compound is administered. In certain embodiments the patient is titrated off standard of care within 12 days from the time the first dose of the PTH compound is administered. In certain embodiments the patient is titrated off standard of care within 10 days from the time the first dose of the PTH compound is administered.

In certain embodiments administration of the PTH compound is by injection, such as be intramuscular, intravenous or subcutaneous injection. In certain embodiments administration is by intramuscular injection. In certain embodiments administration is by intravenous injection. In certain embodiments administration is by subcutaneous injection.

In certain embodiments administration is with a syringe. In certain embodiments administration is with a pen injector. In certain embodiments is with an auto injector.

In certain embodiments the patient, such as a mammalian patient, is selected from mouse, rat, non-human primate and human. In certain embodiments the patient is a human patient. In certain embodiments the patient is a child. In certain embodiments the patient is an adult.

In certain embodiments the single daily dose of the PTH compound administered to the patient is below 31 μg/day. In certain embodiments the single daily dose of the PTH compound administered to the patient is 30 μg/day. In certain embodiments the single daily dose of the PTH compound administered to the patient is 27 μg/day. In certain embodiments the single daily dose of the PTH compound administered to the patient is 24 μg/day. In certain embodiments the single daily dose of the PTH compound administered to the patient is 18 μg/day. In certain embodiments the single daily dose of the PTH compound administered to the patient is 15 μg/day. In certain embodiments the single daily dose of the PTH compound administered to the patient is 12 μg/day. In certain embodiments the single daily dose of the PTH compound administered to the patient is 9 μg/day. In certain embodiments the single daily dose of the PTH compound administered to the patient is 6 μg/day. All doses are provided as PTH equivalents. It is understood that the amount of PTH compound administered to a patient depends on the patient and the severity of the disease.

In certain embodiments the daily dose of the PTH compound administered to the patient is adjusted in response to serum calcium levels, e.g. to avoid hypocalcemia. If serum calcium levels are adequate, no adjustments of the daily dose of the PTH compound may be necessary.

In certain embodiments a patient undergoing the treatment of hypoparathyroidism of the present invention achieves 4 weeks after administration of the first dose of the PTH compound a statistically significant change in the Short Form-36 Physical Component Summary (SF-36 PCS), in the SF-36 Mental Component Summary (MCS) or both the SF-36 PCS and SF-36 MCS. Such change is measured from the respective baseline (BL). As used herein the term “baseline” refers to the numeric SF-36 PCS or SF-36 MCS scores in the respective patient or group of patients before the start of the treatment. A change is statistically significant if the p-value is 0.05 or lower. In certain embodiments such statistically significant change from BL is a change of at least 3, particularly of at least 4.

Both the SF-36 PCS and SF-36 MCS score are generated using a normative scoring system with a score of 50 as the norm for the general population. Unless stated otherwise, numbers are not placebo-adjusted, i.e. the change in the respective SF-36 Component achieved in the placebo group during the same time period is not subtracted.

In certain embodiments the SF-36 PCS improves by at least 4 (not placebo-adjusted) or at least 5 (placebo-adjusted). In certain embodiments the SF-36 MCS improves by at least 5 (not placebo-adjusted) or at least 6 (placebo-adjusted), such as at least 7 or at least 8.

In certain embodiments the PTH compound comprises a PTH moiety having the sequence of SEQ ID NO:47, SEQ ID NO:48, SEQ ID NO:49, SEQ ID NO:50, SEQ ID NO:51, SEQ ID NO:52, SEQ ID NO:53, SEQ ID NO:54, SEQ ID NO:55, SEQ ID NO:107, SEQ ID NO:108, SEQ ID NO:109, SEQ ID NO:110, SEQ ID NO:111, SEQ ID NO:112, SEQ ID NO:113, SEQ ID NO: 114 or SEQ ID NO: 115. More preferably the PTH moiety has the sequence of SEQ ID NO:50, SEQ ID NO:51, SEQ ID NO:52, SEQ ID NO:110, SEQ ID NO:111 or SEQ ID NO: 112. In certain embodiments the PTH moiety has the sequence of SEQ ID NO:50. In certain embodiment the PTH moiety has the sequence of SEQ ID NO:52. In certain embodiments the PTH moiety has the sequence of SEQ ID NO:110. In certain embodiments the PTH moiety has the sequence of SEQ ID NO:111. In certain embodiments PTH moiety has the sequence of SEQ ID NO: 112. In certain embodiments PTH moiety has the sequence of SEQ ID NO:51.

In certain embodiments the PTH compound is water-soluble.

In certain embodiment the PTH compound is a conjugate or a pharmaceutically acceptable salt thereof comprising at least one moiety -D conjugated via at least one moiety -L¹-L²- to at least one moiety Z, wherein the linkage between -D and -L¹- is reversible and wherein a moiety -L²- is conjugated to Z, wherein each -D is independently a PTH moiety; each -L¹- is independently a reversible linker moiety; each -L²- is independently a single chemical bond or a spacer moiety; and each Z is independently a polymeric moiety or a C₈₋₂₄ alkyl moiety.

In certain embodiments the PTH compound is a compound of formula (Ia) or (Ib) or a pharmaceutically acceptable salt thereof

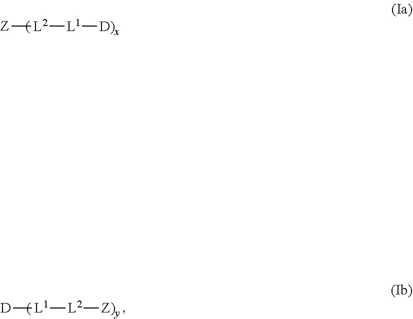

wherein
-D is a PTH moiety;
-L¹- is a linker moiety reversibly and covalently connected to the PTH moiety -D through a functional group of PTH;
-L²- is a single chemical bond or a spacer moiety;
—Z is a polymer moiety or a C₈₋₂₄ alkyl moiety;
x is an integer selected from the group consisting of 1, 2, 3, 4, 5, 6, 7, 8, 9, 10, 11, 12, 13, 14, 15 or 16; and
y is an integer selected from the group consisting of 2, 3, 4 and 5.

It is understood that the compounds of formula (Ia) and (Ib) are PTH prodrugs. Such PTH prodrugs are controlled-release PTH compounds.

In certain embodiments the PTH compound is of formula (Ia). In certain embodiments the PTH compound is of formula (Ib).

In certain embodiments -D has the sequence of SEQ ID NO:47, SEQ ID NO:48, SEQ ID NO:49, SEQ ID NO:50, SEQ ID NO:51, SEQ ID NO:52, SEQ ID NO:53, SEQ ID NO:54, SEQ ID NO:55, SEQ ID NO:107, SEQ ID NO:108, SEQ ID NO:109, SEQ ID NO:110, SEQ ID NO:111, SEQ ID NO:112, SEQ ID NO:113, SEQ ID NO:114 or SEQ ID NO:115. In certain embodiments -D has the sequence of SEQ ID NO:50, SEQ ID NO:51, SEQ ID NO:52, SEQ ID NO:110, SEQ ID NO:111 or SEQ ID NO:112. In certain embodiments -D has the sequence of SEQ ID NO:50. In certain embodiments -D has the sequence of SEQ ID NO:52. In certain embodiments -D has the sequence of SEQ ID NO:110. In certain embodiments -D has the sequence of SEQ ID NO:111. In certain embodiments -D has the sequence of SEQ ID NO:112. In certain embodiments -D has the sequence of SEQ ID NO:51.

The moiety -L¹- is either conjugated to a functional group of the side chain of an amino acid residue of -D, to the N-terminal amine functional group or to the C-terminal carboxyl functional group of -D or to a nitrogen atom in the backbone polypeptide chain of -D. Attachment to either the N-terminus or C-terminus can either be directly through the corresponding amine or carboxyl functional group, respectively, or indirectly wherein a spacer moiety is first conjugated to the amine or carboxyl functional group to which spacer moiety -L¹- is conjugated. In certain embodiments -L¹- is conjugated to a functional group of the side chain of an amino acid residue of -D. In certain embodiments -L¹- is conjugated to the N-terminal amine functional group. In certain embodiments -L¹- is conjugated to the C-terminal carboxyl functional group. In certain embodiments -L¹- is conjugated to a nitrogen atom in the backbone polypeptide chain of -D.

In certain embodiments the amino acid residue of PTH to which -L¹- is conjugated comprises a functional group selected from the group consisting carboxylic acid, primary and secondary amine, maleimide, thiol, sulfonic acid, carbonate, carbamate, hydroxyl, aldehyde, ketone, hydrazine, isocyanate, isothiocyanate, phosphoric acid, phosphonic acid, haloacetyl, alkyl halide, acryloyl, aryl fluoride, hydroxylamine, sulfate, disulfide, vinyl sulfone, vinyl ketone, diazoalkane, oxirane, guanidine and aziridine. In certain embodiments the amino acid residue of PTH to which -L¹- is conjugated comprises a functional group selected from the group consisting hydroxyl, primary and secondary amine and guanidine. In certain embodiments the amino acid residue of PTH to which -L¹- is conjugated comprises a primary or secondary amine functional group. In certain embodiments the amino acid residue of PTH to which -L¹- is conjugated comprises a primary amine functional group.

If the moiety -L¹- is conjugated to a functional group of the side chain of an amino acid residue of PTH said amino acid residue may be selected from the group consisting of proteinogenic amino acid residues and non-proteinogenic amino acid residues. In certain embodiments -L¹- is conjugated to a functional group of the side chain of a non-proteinogenic amino acid residue of PTH. It is understood that such non-proteinogenic amino acid is not found in the sequence of native PTH or fragments thereof and that it may only be present in variants and derivatives of PTH. In certain embodiments -L¹- is conjugated to a functional group of the side chain of a proteinogenic amino acid residue of PTH, such as an amino acid selected from the group consisting of histidine, lysine, tryptophan, serine, threonine, tyrosine, aspartic acid, glutamic acid and arginine. In certain embodiments said amino acid is selected from the group consisting of lysine, aspartic acid, arginine and serine. In certain embodiments said amino acid is selected from the group consisting of lysine, arginine and serine. In certain embodiments -L¹- is conjugated to a functional group of the side chain of a histidine of PTH. In certain embodiments -L¹- is conjugated to a functional group of the side chain of a lysine of PTH. In certain embodiments -L¹- is conjugated to a functional group of the side chain of a tryptophan of PTH. In certain embodiments -L¹- is conjugated to a functional group of the side chain of a serine of PTH. In certain embodiments -L¹- is conjugated to a functional group of the side chain of a threonine of PTH. In certain embodiments -L¹- is conjugated to a functional group of the side chain of a tyrosine of PTH. In certain embodiments -L¹- is conjugated to a functional group of the side chain of an aspartic acid of PTH. In certain embodiments -L¹- is conjugated to a functional group of the side chain of a glutamic acid of PTH. In certain embodiments -L¹- is conjugated to a functional group of the side chain of an arginine of PTH. It is understood that not every PTH moiety may comprise all of these amino acid residues.

In certain embodiments -L¹- is conjugated to the N-terminal amine functional group of PTH, either directly through the corresponding amine functional group or indirectly wherein a spacer moiety is first conjugated to the amine functional group to which spacer moiety -L¹- is conjugated. In certain embodiments -L¹- is directly conjugated to the N-terminal amine functional group of PTH.

In certain embodiments -L¹- is conjugated to the C-terminal functional group of PTH, either directly through the corresponding carboxyl functional group or indirectly wherein a spacer moiety is first conjugated to the carboxyl functional group to which spacer moiety -L¹- is conjugated.

In certain embodiments -L¹- is directly conjugated to the N-terminal amine functional group of PTH.

The moiety -L¹- may be connected to -D through any type of linkage, provided that it is reversible. In certain embodiments -L¹- is connected to -D through a linkage selected from the group consisting of amide, ester, carbamate, acetal, aminal, imine, oxime, hydrazone, disulfide and acylguanidine. In certain embodiments -L¹- is connected to -D through a linkage selected from the group consisting of amide, ester, carbamate and acylguanidin. It is understood that some of these linkages may not reversible per se, but that in the present invention neighboring groups of -L¹- render these linkages reversible.

In certain embodiments -L¹- is connected to -D through an ester linkage. In certain embodiments -L¹- is connected to -D through a carbamate linkage. In certain embodiments -L¹- is connected to -D through an acylguanidine. In certain embodiments -L¹- is connected to -D through an amide linkage.

The moiety -L¹- is a reversible linker from which the drug, i.e. PTH, is released in its free form, i.e. it is a traceless linker. Suitable reversible linkers are known in the art, such as for example the reversible linker moieties disclosed in WO 2005/099768 A2, WO 2006/136586 A2, WO 2011/089216 A1 and WO 2013/024053 A1, which are incorporated by reference herewith.

In certain embodiments -L¹- is a reversible prodrug linker as described in WO 2011/012722 A1, WO 2011/089214 A1, WO 2011/089215 A1, WO 2013/024052 A1 and WO 2013/160340 A1 which are incorporated by reference herewith.

In certain embodiments -L¹- is disclosed in WO 2009/095479 A2. Accordingly, in certain embodiments -L¹- is of formula (II):

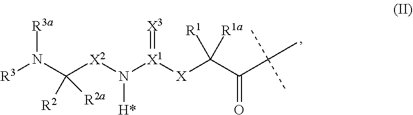

wherein the dashed line indicates attachment to a nitrogen, hydroxyl or thiol of -D which is a PTH moiety;
—X— is selected from the group consisting of —C(R⁴R^{4a})—; —N(R⁴)—; —O—; —C(R⁴R^{4a})—C(R⁵R^{5a})—; —C(R⁵R^{5a})—C(R⁴R^{4a})—; —C(R⁴R^{4a})—N(R⁶)—; —N(R⁶)—C(R⁴R^{4a})—; —C(R⁴R^{4a})—O—; —O—C(R⁴R^{4a})—; and —C(R⁷R^{7a})—;
X¹ is selected from the group consisting of C; and S(O);
—X²— is selected from the group consisting of —C(R⁸R^{8a})—; and —C(R⁸R^{8a})—C(R⁹R^{9a})—;
═X³ is selected from the group consisting of ═O; ═S; and ═N—CN;
—R¹, —R^{1a}, —R², —R^{2a}, —R⁴, —R^{4a}, —R⁵, —R^{5a}, —R⁶, —R⁸, —R^{8a}, —R⁹ and —R^{9a} are independently selected from the group consisting of —H; and C₁₋₆ alkyl;
—R³ and —R^{3a} are independently selected from the group consisting of —H; and C₁₋₆ alkyl, provided that in case one of —R³ and —R^{3a} or both are other than —H they are connected to N to which they are attached through an sp³-hybridized carbon atom;
—R⁷ is selected from the group consisting of —N(R¹⁰R^{10a}); and —NR¹⁰—(C═O)—R¹¹;
—R^{7a} —R¹⁰, —R^{10a} and —R¹¹ are independently of each other selected from the group consisting of —H; and C₁₋₆ alkyl;
optionally, one or more of the pairs —R^{1a}/—R^{4a}, —R^{1a}/—R^{5a}, —R^{1a}/—R^{7a}, —R^{4a}/—R^{5a} and —R^{8a}/—R^{9a} form a chemical bond;
optionally, one or more of the pairs —R¹/—R^{1a}, —R²/—R^{2a}, —R⁴/—R^{4a}, —R⁵/—R^{5a}, —R⁸/—R^{8a} and —R⁹/—R^{9a} are joined together with the atom to which they are attached to form a C₃₋₁₀ cycloalkyl; or 3- to 10-membered heterocyclyl;
optionally, one or more of the pairs —R¹/—R⁴, —R¹/—R⁵, —R¹/—R⁶, —R¹/—R^{7a}, —R⁴/—R⁵, —R⁴/—R⁶, —R⁸/—R⁹ and —R²/—R³ are joined together with the atoms to which they are attached to form a ring A;
optionally, R³/R^{3a} are joined together with the nitrogen atom to which they are attached to form a 3- to 10-membered heterocycle;
A is selected from the group consisting of phenyl; naphthyl; indenyl; indanyl; tetralinyl; C₃₋₁₀ cycloalkyl; 3- to 10-membered heterocyclyl; and 8- to 11-membered heterobicyclyl; and
wherein -L¹- is substituted with -L²-Z and wherein -L¹- is optionally further substituted, provided that the hydrogen marked with the asterisk in formula (II) is not replaced by -L²-Z or a substituent;
   wherein
   -L²- is a single chemical bond or a spacer; and
   —Z is a water-soluble carrier.

In certain embodiments -L¹- of formula (II) is substituted with one moiety -L²-Z.

In certain embodiments -L¹- of formula (II) is not further substituted.

It is understood that if —R³/—R^{3a} of formula (II) are joined together with the nitrogen atom to which they are attached to form a 3- to 10-membered heterocycle, only such 3- to 10-membered heterocycles may be formed in which the atoms directly attached to the nitrogen are sp³-hybridized carbon atoms. In other words, such 3- to 10-membered heterocycle formed by —R³/—R^{3a} together with the nitrogen atom to which they are attached has the following structure:

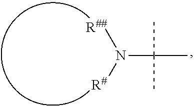

wherein
the dashed line indicates attachment to the rest of -L¹-;
the ring comprises 3 to 10 atoms comprising at least one nitrogen; and
R^{#} and R^{##} represent an sp³-hybridized carbon atom.

It is also understood that the 3- to 10-membered heterocycle may be further substituted.

Exemplary embodiments of suitable 3- to 10-membered heterocycles formed by —R³/—R^{3a} of formula (II) together with the nitrogen atom to which they are attached are the following:

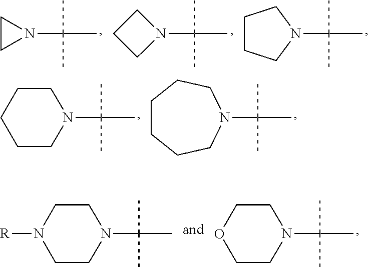

wherein
dashed lines indicate attachment to the rest of the molecule; and
—R is selected from the group consisting of —H and C₁₋₆ alkyl.

-L¹- of formula (II) may optionally be further substituted. In general, any substituent may be used as far as the cleavage principle is not affected, i.e. the hydrogen marked with the asterisk in formula (II) is not replaced and the nitrogen of the moiety

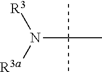

of formula (II) remains part of a primary, secondary or tertiary amine, i.e. —R³ and —R^{3a} are independently of each other —H or are connected to —N< through an sp³-hybridized carbon atom.

In certain embodiments —R¹ or —R^{1a} of formula (II) is substituted with -L²-Z. In certain embodiments —R² or —R^{2a} of formula (II) is substituted with -L²-Z. In certain embodiments —R³ or —R^{3a} of formula (II) is substituted with -L²-Z. In certain embodiments —R⁴ of formula (II) is substituted with -L²-Z. In certain embodiments —R⁵ or —R^{5a} of formula (II) is substituted with -L²-Z. In certain embodiments —R⁶ of formula (II) is substituted with -L²-Z. In certain embodiments —R⁷ or —R^{7a} of formula (II) is substituted with -L²-Z. In certain embodiments —R⁸ or —R^{8a} of formula (II) is substituted with -L²-Z. In certain embodiments —R⁹ or —R^{9a} of formula (II) is substituted with -L²-Z. In certain embodiments —R¹⁰ is substituted with -L²-Z. In certain embodiments —R¹¹ is substituted with -L²-Z.

In certain embodiments —X— of formula (II) is selected from the group consisting of —C(R⁴R^{4a})—, —N(R⁴)— and —C(R⁷R^{7a})—. In certain embodiments —X— of formula (II) is —C(R⁴R^{4a})—. In certain embodiments —X— of formula (II) is —C(R⁷R^{7a})—.

In certain embodiments —R⁷ of formula (II) is —NR¹⁰—(C═O)—R¹¹.

In certain embodiments —R^{7a} of formula (II) is selected from —H, methyl and ethyl. In certain embodiments —R^{7a} of formula (II) is —H.

In certain embodiments —R¹⁰ is selected from —H, methyl and ethyl. In certain embodiments —R¹⁰ is methyl.

In certain embodiments —R¹¹ is selected from —H, methyl and ethyl. In certain embodiments —R¹¹ is —H.

In certain embodiments —R¹¹ is substituted with -L²-Z.

In certain embodiments —X— of formula (II) is —N(R⁴)—.

In certain embodiments —R⁴ is selected from the group consisting of —H, methyl and ethyl. In certain embodiments —R⁴ is —H.

In certain embodiments X¹ of formula (II) is C.

In certain embodiments ═X³ of formula (II) is ═O.

In certain embodiments —X²— of formula (II) is —C(R⁸R^{8a})—.

In certain embodiments —R⁸ and —R^{8a} of formula (II) are independently selected from the group consisting of —H, methyl and ethyl. In certain embodiments at least one of —R⁸ and —R^{8a} of formula (II) is —H. In certain embodiments both —R⁸ and —R^{8a} of formula (II) are —H.

In certain embodiments —R¹ and —R^{1a} of formula (II) are independently selected from the group consisting of —H, methyl and ethyl.

In certain embodiments at least one of —R¹ and —R^{1a} of formula (II) is —H, more preferably both —R¹ and —R^{1a} of formula (II) are —H.

In certain embodiments at least one of —R¹ and —R^{1a} of formula (II) is methyl, In certain embodiments both —R¹ and —R^{1a} of formula (II) are methyl.

In certain embodiments —R² and —R^{2a} of formula (II) are independently selected from the group consisting of —H, methyl and ethyl. In certain embodiments at least one of —R² and —R^{2a} of formula (II) is —H. In certain embodiments both —R² and —R^{2a} of formula (II) are H.

In certain embodiments —R³ and —R^{3a} of formula (II) are independently selected from the group consisting of —H, methyl, ethyl, propyl and butyl.

In certain embodiments at least one of —R³ and —R^{3a} of formula (II) is methyl, In certain embodiments —R³ of formula (II) is methyl and —R^{3a} of formula (II) is —H.

In certain embodiments —R³ and —R^{3a} of formula (II) are both —H.

In certain embodiments -D is connected to -L¹- of formula (II) through a nitrogen by forming an amide bond.

In certain embodiments the moiety -L¹- is of formula (IIa-i)

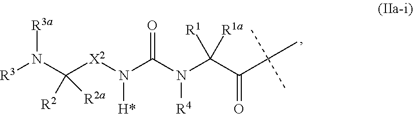

wherein
the dashed line indicates the attachment to a nitrogen of -D which is a PTH moiety by forming an amide bond;
—R¹, —R^{1a}, —R², —R^{2a}, —R³, —R^{3a}, —R⁴ and —X²— are used as defined in formula (II); and
wherein -L¹- is substituted with -L²-Z and wherein -L¹- is optionally further substituted, provided that the hydrogen marked with the asterisk in formula (IIa-i) is not replaced by -L²-Z or a substituent.

It is understood that in case one of —R³, —R^{3a} of formula (IIa-i) or both are other than —H they are connected to N to which they are attached through an sp³-hybridized carbon atom.

In certain embodiments -L¹- of formula (IIa-i) is substituted with one moiety -L²-Z.

In certain embodiments the moiety -L¹- of formula (IIa-i) is not further substituted.

In certain embodiments —R¹ and —R^{1a} of formula (IIa-i) are independently selected from the group consisting of —H, methyl and ethyl. In certain embodiments at least one of —R¹ and —R^{1a} of formula (IIa-i) is methyl. In certain embodiments both —R¹ and —R^{1a} of formula (IIa-i) are methyl.

In certain embodiments —R⁴ of formula (IIa-i) is selected from the group consisting of —H, methyl and ethyl. In certain embodiments —R⁴ of formula (IIa-i) is —H.

In certain embodiments —X²— of formula (IIa-i) is —C(R⁸R^{8a})—.

In certain embodiments —R⁸ and —R^{8a} of formula (IIa-i) are independently selected from the group consisting of —H, methyl and ethyl. In certain embodiments at least one of —R⁸ and —R^{8a} of formula (IIa-i) is —H. In certain embodiments both —R⁸ and —R^{8a} of formula (IIa-i) are —H.

In certain embodiments —R² and —R^{2a} of formula (IIa-i) are independently selected from the group consisting of —H, methyl and ethyl. In certain embodiments, at least one of —R² and —R^{2a} of formula (IIa-i) is —H. In certain embodiments both —R² and —R^{2a} of formula (IIa-i) are H.

In certain embodiments —R³ and —R^{3a} of formula (IIa-i) are independently selected from the group consisting of —H, methyl, ethyl, propyl and butyl. In certain embodiments at least one of —R³ and —R^{3a} of formula (IIa-i) is —H. In certain embodiments both —R³ and —R^{3a} of formula (IIa-i) are —H.

In certain embodiments the moiety -L¹- is of formula (IIa-ii):

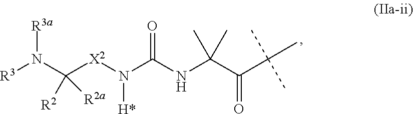

wherein the dashed line indicates the attachment to a nitrogen of -D which is a PTH moiety by forming an amide bond;
—R², —R^{2a}, —R³, —R^{3a} and —X²— are used as defined in formula (II); and
wherein -L¹- is substituted with -L²-Z and wherein -L¹- is optionally further substituted, provided that the hydrogen marked with the asterisk in formula (IIa-ii) is not replaced by -L²-Z or a substituent.

It is understood that in case one of —R³, —R^{3a} of formula (IIa-ii) or both are other than —H they are connected to N to which they are attached through an sp³-hybridized carbon atom.

In certain embodiments -L¹- of formula (IIa-ii) is substituted with one moiety -L²-Z.

In certain embodiments the moiety -L¹- of formula (IIa-ii) is not further substituted.

In certain embodiments —X²— of formula (IIa-ii) is —C(R⁸R^{8a})—.

In certain embodiments —R⁸ and —R^{8a} of formula (IIa-ii) are independently selected from the group consisting of —H, methyl and ethyl. In certain embodiments at least one of —R⁸ and —R^{8a} of formula (IIa-ii) is —H. In certain embodiments both —R⁸ and —R^{8a} of formula (IIa-ii) are —H.

In certain embodiments —R² and —R^{2a} of formula (IIa-ii) are independently selected from the group consisting of —H, methyl and ethyl. In certain embodiments at least one of —R² and —R^{2a} of formula (IIa-ii) is —H. In certain embodiments both —R² and —R^{2a} of formula (IIa-ii) are H.

In certain embodiments —R³ and —R^{3a} of formula (IIa-ii) are independently selected from the group consisting of —H, methyl, ethyl, propyl and butyl. In certain embodiments at least one of —R³ and —R^{3a} of formula (IIa-ii) is —H. In certain embodiments both —R³ and —R^{3a} of formula (IIa-ii) are —H.

In certain embodiments the moiety -L¹- is of formula (IIa-ii′):

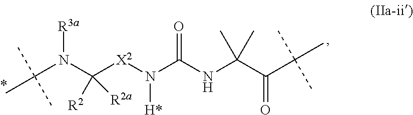

wherein
the dashed line indicates the attachment to a nitrogen of -D which is a PTH moiety by forming an amide bond;
—R², —R^{2a}, —R^{3a} and —X²— are used as defined in formula (II); and
wherein -L¹- is substituted with -L²-Z and wherein -L¹- is optionally further substituted, provided that the hydrogen marked with the asterisk in formula (IIa-ii′) is not replaced by -L²-Z or a substituent.

It is understood that in case —R^{3a} of formula (IIa-ii′) is other than —H it are connected to N to which it is attached through an sp³-hybridized carbon atom.

In certain embodiments the moiety -L¹- of formula (IIa-ii′) is not further substituted.

In certain embodiments —X²— of formula (IIa-ii′) is —C(R⁸R^{8a})—.

In certain embodiments —R⁸ and —R^{8a} of formula (IIa-ii′) are independently selected from the group consisting of —H, methyl and ethyl. In certain embodiments at least one of —R⁸ and —R^{8a} of formula (IIa-ii′) is —H. In certain embodiments both —R⁸ and —R^{8a} of formula (IIa-ii′) are —H.

In certain embodiments —R² and —R^{2a} of formula (IIa-ii′) are independently selected from the group consisting of —H, methyl and ethyl. In certain embodiments at least one of —R² and —R^{2a} of formula (IIa-ii′) is —H. In certain embodiments both —R² and —R^{2a} of formula (IIa-ii′) are H.

In certain embodiments —R^{3a} of formula (IIa-ii′) is selected from the group consisting of —H, methyl, ethyl, propyl and butyl. In certain embodiments —R^{3a} of formula (IIa-ii′) is —H.

In certain embodiments the moiety -L¹- is of formula (IIa-iii):

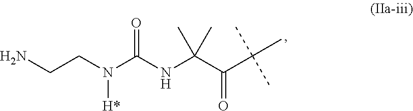

wherein
the dashed line indicates the attachment to a nitrogen of -D which is a PTH moiety by forming an amide bond; and
wherein -L¹- is substituted with -L²-Z and wherein -L¹- is optionally further substituted, provided that the hydrogen marked with the asterisk in formula (IIa-iii) is not replaced by -L²-Z or a substituent.

It is understood that in case one of —R³, —R^{3a} of formula (IIa-iii) or both are other than —H they are connected to N to which they are attached through an sp³-hybridized carbon atom.

In certain embodiments -L¹- of formula (IIa-iii) is substituted with one moiety -L²-Z.

In certain embodiments the moiety -L¹- of formula (IIa-iii) is not further substituted.

In certain embodiments the moiety -L¹- is of formula (IIa-iii′):

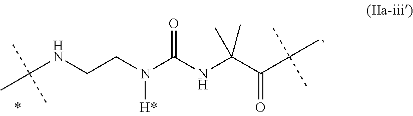

wherein
the dashed line indicates the attachment to a nitrogen of -D which is a PTH moiety by forming an amide bond;
the dashed line marked with the asterisk indicates attachment to -L²-; and
wherein -L¹- is optionally further substituted, provided that the hydrogen marked with the asterisk in formula (IIa-iii′) is not replaced by -L²-Z or a substituent.

It is understood that the nitrogen adjacent to the dashed line marked with the asterisk in formula (IIa-iii′) is attached to -L²- through an sp³-hybridized carbon atom.

In certain embodiments the moiety -L¹- of formula (IIa-iii′) is not further substituted.

In certain embodiments moiety -L¹- is disclosed in WO2016/020373A1. Accordingly, In certain embodiments the moiety -L¹- is of formula (III):

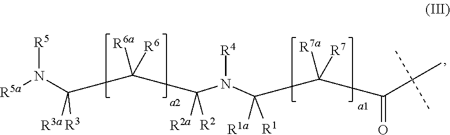

wherein
the dashed line indicates attachment to a primary or secondary amine or hydroxyl of -D which is a PTH moiety by forming an amide or ester linkage, respectively;
—R¹, —R^{1a}, —R², —R^{2a}, —R³ and —R^{3a} are independently of each other selected from the group consisting of —H, —C(R⁸R^{8a}R^{8b}), —C(═O)R⁸, —C≡N, —C(═NR⁸)R^{8a}, —CR⁸(═CR^{8a}R^{8b}), —C≡CR^{B} and -T;
—R⁴, —R⁵ and —R^{5a} are independently of each other selected from the group consisting of —H, —C(R⁹R^{9a}R^{9b}) and -T;
a1 and a2 are independently of each other 0 or 1;
each —R⁶, —R^{6a}, —R⁷, —R^{7a}, —R⁸, —R^{8a}, —R^{8b}, —R⁹, —R^{9a}, —R⁹ are independently of each other selected from the group consisting of —H, halogen, —CN, —COOR¹⁰, —OR¹⁰, —C(O)R¹⁰, —C(O)N(R¹⁰R^{10a}), —S(O)₂N(R¹⁰R^{10a}), —S(O)N(R¹⁰R^{10a}), —S(O)₂R¹⁰, —S(O)R¹⁰, —N(R¹⁰)S(O)₂N(R^{10a}R^{10b}), —SR¹⁰, —N(R¹⁰R^{10a}), —NO₂, —OC(O)R¹⁰, —N(R¹⁰)C(O)R^{10a}, —N(R¹⁰)S(O)₂R^{10a}, —N(R¹⁰)S(O)R^{10a}, —N(R¹⁰)C(O)OR^{10a}, —N(R¹⁰)C(O)N(R^{10a}R^{10b}), —OC(O)N(R¹⁰R^{10a}), -T, C₁₋₂₀ alkyl, C₂₋₂₀ alkenyl, and C₂₋₂₀ alkynyl; wherein -T, C₁₋₂₀ alkyl, C₂₋₂₀ alkenyl, and C₂₋₂₀ alkynyl are optionally substituted with one or more —R¹¹, which are the same or different and wherein C₁₋₂₀ alkyl, C₂₋₂₀ alkenyl, and C₂₋₂₀ alkynyl are optionally interrupted by one or more groups selected from the group consisting of -T-, —C(O)O—, —O—, —C(O)—, —C(O)N(R¹²)—, —S(O)₂N(R¹²)—, —S(O)N(R¹²)—, —S(O)₂—, —S(O)—, —N(R¹²)S(O)₂N(R^{12a})—, —S—, —N(R¹²)—, —OC(OR¹²)(R^{12a})—, —N(R¹²)C(O)N(R^{12a})—, and —OC(O)N(R¹²)—;
each —R¹⁰, —R^{10a}, —R^{10b} is independently selected from the group consisting of —H, -T, C₁₋₂₀ alkyl, C₂₋₂₀ alkenyl, and C₂₋₂₀ alkynyl; wherein -T, C₁₋₂₀ alkyl, C₂₋₂₀ alkenyl, and C₂₋₂₀ alkynyl are optionally substituted with one or more —R¹¹, which are the same or different and wherein C₁₋₂₀ alkyl, C₂₋₂₀ alkenyl, and C₂₋₂₀ alkynyl are optionally interrupted by one or more groups selected from the group consisting of -T-, —C(O)O—, —O—, —C(O)—, —C(O)N(R¹²)—, —S(O)₂N(R¹²)—, —S(O)N(R¹²)—, —S(O)₂—, —S(O)—, —N(R¹²)S(O)₂N(R^{12a})—, —S—, —N(R¹²)—, —OC(OR¹²)(R^{12a})—, —N(R¹²)C(O)N(R^{12a})—, and —OC(O)N(R¹²)—;
each T is independently of each other selected from the group consisting of phenyl, naphthyl, indenyl, indanyl, tetralinyl, C₃₋₁₀ cycloalkyl, 3- to 10-membered heterocyclyl, and 8- to 11-membered heterobicyclyl; wherein each T is independently optionally substituted with one or more —R¹¹, which are the same or different;
each —R¹¹ is independently of each other selected from halogen, —CN, oxo (═O), —COOR¹³, —OR¹³, —C(O)R¹³, —C(O)N(R¹³R^{13a}), —S(O)₂N(R¹³R^{13a}), —S(O)N(R¹³R^{13a}), —S(O)₂R¹³, —S(O)R¹³, —N(R¹³)S(O)₂N(R^{13a}R^{13b}), —SR¹³, —N(R¹³R^{13a}), —NO₂, —OC(O)R¹³, —N(R¹³)C(O)R^{13a}, —N(R¹³)S(O)₂R^{13a}, —N(R¹³)S(O)R^{13a}, —N(R¹³)C(O)OR^{13a}, —N(R¹³)C(O)N(R^{13a}R^{13b}), —OC(O)N(R¹³R^{13a}), and C₁₋₆ alkyl; wherein C₁₋₆ alkyl is optionally substituted with one or more halogen, which are the same or different;
each —R¹², —R^{12a}, —R¹³, —R^{13a}, —R^{13b} is independently selected from the group consisting of —H, and C₁₋₆ alkyl; wherein C₁₋₆ alkyl is optionally substituted with one or more halogen, which are the same or different;
optionally, one or more of the pairs -R/—R^{1a}, —R²/—R^{2a}, —R³/—R^{3a}, —R⁶/—R^{6a}, —R⁷/—R^{7a} are joined together with the atom to which they are attached to form a C₃₋₁₀ cycloalkyl or a 3- to 10-membered heterocyclyl;
optionally, one or more of the pairs —R¹/—R², —R¹/—R³, —R¹/—R⁴, —R¹/—R⁵, —R¹/—R⁶, —R¹/—R⁷, —R²/—R³, —R²/—R⁴, —R²/—R⁵, —R²/—R⁶, —R²/—R⁷, —R³/—R⁴, —R³/—R⁵, —R³/—R⁶, —R³/—R⁷, —R⁴/—R⁵, —R⁴/—R⁶, —R⁴/—R⁷, —R⁵/—R⁶, —R⁵/—R⁷, —R⁶/—R⁷ are joint together with the atoms to which they are attached to form a ring A;
A is selected from the group consisting of phenyl; naphthyl; indenyl; indanyl; tetralinyl; C₃₋₁₀ cycloalkyl; 3- to 10-membered heterocyclyl; and 8- to 11-membered heterobicyclyl;
wherein -L¹- is substituted with -L²-Z and wherein -L¹- is optionally further substituted;
   wherein
   -L²- is a single chemical bond or a spacer; and
   —Z is a water-soluble carrier.

The optional further substituents of -L¹- of formula (III) are preferably as described above.

In certain embodiments -L¹- of formula (III) is substituted with one moiety -L²-Z.

In certain embodiments -L¹- of formula (III) is not further substituted.

More embodiments for -L¹- are disclosed in EP1536334B1, WO2009/009712A1, WO2008/034122A1, WO2009/143412A2, WO2011/082368A2, and U.S. Pat. No. 8,618,124B2, which are herewith incorporated by reference in their entirety.

More embodiments for -L¹- are disclosed in U.S. Pat. No. 8,946,405B2 and U.S. Pat. No. 8,754,190B2, which are herewith incorporated by reference in their entirety. Accordingly, a In certain embodiments moiety -L¹- is of formula (IV):

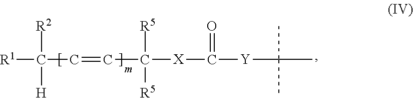

wherein
the dashed line indicates attachment to -D which is a PTH moiety and wherein attachment is through a functional group of -D selected from the group consisting of —OH, —SH and —NH₂;
m is 0 or 1;
at least one or both of —R¹ and —R² is/are independently of each other selected from the group consisting of —CN, —NO₂, optionally substituted aryl, optionally substituted heteroaryl, optionally substituted alkenyl, optionally substituted alkynyl, —C(O)R³, —S(O)R³, —S(O)₂R³, and —SR⁴,
one and only one of —R¹ and —R² is selected from the group consisting of —H, optionally substituted alkyl, optionally substituted arylalkyl, and optionally substituted heteroarylalkyl;
—R³ is selected from the group consisting of —H, optionally substituted alkyl, optionally substituted aryl, optionally substituted arylalkyl, optionally substituted heteroaryl, optionally substituted heteroarylalkyl, —OR⁹ and —N(R⁹)₂;
—R⁴ is selected from the group consisting of optionally substituted alkyl, optionally substituted aryl, optionally substituted arylalkyl, optionally substituted heteroaryl, and optionally substituted heteroarylalkyl;
each —R⁵ is independently selected from the group consisting of —H, optionally substituted alkyl, optionally substituted alkenylalkyl, optionally substituted alkynylalkyl, optionally substituted aryl, optionally substituted arylalkyl, optionally substituted heteroaryl and optionally substituted heteroarylalkyl;
—R⁹ is selected from the group consisting of —H and optionally substituted alkyl;
—Y— is absent and —X— is —O— or —S—; or
—Y— is —N(Q)CH₂— and —X— is —O—;
Q is selected from the group consisting of optionally substituted alkyl, optionally substituted aryl, optionally substituted arylalkyl, optionally substituted heteroaryl and optionally substituted heteroarylalkyl;
optionally, —R¹ and —R² may be joined to form a 3 to 8-membered ring; and
optionally, both —R⁹ together with the nitrogen to which they are attached form a heterocyclic ring;
wherein -L¹- is substituted with -L²-Z and wherein -L¹- is optionally further substituted;
   wherein
   -L²- is a single chemical bond or a spacer; and
   —Z is a water-soluble carrier.

Only in the context of formula (IV) the terms used have the following meaning:

The term “alkyl” as used herein includes linear, branched or cyclic saturated hydrocarbon groups of 1 to 8 carbons, or in some embodiments 1 to 6 or 1 to 4 carbon atoms.

The term “alkoxy” includes alkyl groups bonded to oxygen, including methoxy, ethoxy, isopropoxy, cyclopropoxy, cyclobutoxy, and similar.

The term “alkenyl” includes non-aromatic unsaturated hydrocarbons with carbon-carbon double bonds.

The term “alkynyl” includes non-aromatic unsaturated hydrocarbons with carbon-carbon triple bonds.

The term “aryl” includes aromatic hydrocarbon groups of 6 to 18 carbons, preferably 6 to 10 carbons, including groups such as phenyl, naphthyl, and anthracenyl. The term “heteroaryl” includes aromatic rings comprising 3 to 15 carbons containing at least one N, O or S atom, preferably 3 to 7 carbons containing at least one N, O or S atom, including groups such as pyrrolyl, pyridyl, pyrimidinyl, imidazolyl, oxazolyl, isoxazolyl, thiazolyl, isothiazolyl, quinolyl, indolyl, indenyl, and similar.

In some instance, alkenyl, alkynyl, aryl or heteroaryl moieties may be coupled to the remainder of the molecule through an alkylene linkage. Under those circumstances, the substituent will be referred to as alkenylalkyl, alkynylalkyl, arylalkyl or heteroarylalkyl, indicating that an alkylene moiety is between the alkenyl, alkynyl, aryl or heteroaryl moiety and the molecule to which the alkenyl, alkynyl, aryl or heteroaryl is coupled.

The term “halogen” includes bromo, fluoro, chloro and iodo.

The term “heterocyclic ring” refers to a 4 to 8 membered aromatic or non-aromatic ring comprising 3 to 7 carbon atoms and at least one N, O, or S atom. Examples are piperidinyl, piperazinyl, tetrahydropyranyl, pyrrolidine, and tetrahydrofuranyl, as well as the exemplary groups provided for the term “heteroaryl” above.

When a ring system is optionally substituted, suitable substituents are selected from the group consisting of alkyl, alkenyl, alkynyl, or an additional ring, each optionally further substituted.

Optional substituents on any group, including the above, include halo, nitro, cyano, —OR, —SR, —NR₂, —OCOR, —NRCOR, —COOR, —CONR₂, —SOR, —SO₂R, —SONR₂, —SO₂NR₂, wherein each R is independently alkyl, alkenyl, alkynyl, aryl or heteroaryl, or two R groups taken together with the atoms to which they are attached form a ring.

In certain embodiments -L¹- of formula (IV) is substituted with one moiety -L²-Z.

Another embodiment for -L¹- is disclosed in WO2013/036857A1, which is herewith incorporated by reference in its entirety. Accordingly, In certain embodiments -L¹- is of formula (V):

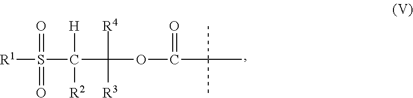

wherein
the dashed line indicates attachment to -D which is a PTH moiety and wherein attachment is through an amine functional group of -D;
—R¹ is selected from the group consisting of optionally substituted C₁-C₆ linear, branched, or cyclic alkyl; optionally substituted aryl; optionally substituted heteroaryl; alkoxy; and —NR⁵₂;
—R² is selected from the group consisting of —H; optionally substituted C₁-C₆ alkyl; optionally substituted aryl; and optionally substituted heteroaryl;
—R³ is selected from the group consisting of —H; optionally substituted C₁-C₆ alkyl; optionally substituted aryl; and optionally substituted heteroaryl;
—R⁴ is selected from the group consisting of —H; optionally substituted C₁-C₆ alkyl; optionally substituted aryl; and optionally substituted heteroaryl;
each —R⁵ is independently of each other selected from the group consisting of —H; optionally substituted C₁-C₆ alkyl; optionally substituted aryl; and optionally substituted heteroaryl; or when taken together two —R⁵ can be cycloalkyl or cycloheteroalkyl;
wherein -L¹- is substituted with -L²-Z and wherein -L¹- is optionally further substituted;
   wherein
   -L²- is a single chemical bond or a spacer; and
   —Z is a water-soluble carrier.

Only in the context of formula (V) the terms used have the following meaning:

“Alkyl”, “alkenyl”, and “alkynyl” include linear, branched or cyclic hydrocarbon groups of 1-8 carbons or 1-6 carbons or 1-4 carbons wherein alkyl is a saturated hydrocarbon, alkenyl includes one or more carbon-carbon double bonds and alkynyl includes one or more carbon-carbon triple bonds. Unless otherwise specified these contain 1-6 C.

“Aryl” includes aromatic hydrocarbon groups of 6-18 carbons, preferably 6-10 carbons, including groups such as phenyl, naphthyl, and anthracene “Heteroaryl” includes aromatic rings comprising 3-15 carbons containing at least one N, O or S atom, preferably 3-7 carbons containing at least one N, O or S atom, including groups such as pyrrolyl, pyridyl, pyrimidinyl, imidazolyl, oxazolyl, isoxazolyl, thiazolyl, isothiazolyl, quinolyl, indolyl, indenyl, and similar.

The term “substituted” means an alkyl, alkenyl, alkynyl, aryl, or heteroaryl group comprising one or more substituent groups in place of one or more hydrogen atoms. Substituents may generally be selected from halogen including F, Cl, Br, and I; lower alkyl including linear, branched, and cyclic; lower haloalkyl including fluoroalkyl, chloroalkyl, bromoalkyl, and iodoalkyl; OH; lower alkoxy including linear, branched, and cyclic; SH; lower alkylthio including linear, branched and cyclic; amino, alkylamino, dialkylamino, silyl including alkylsilyl, alkoxysilyl, and arylsilyl; nitro; cyano; carbonyl; carboxylic acid, carboxylic ester, carboxylic amide, aminocarbonyl; aminoacyl; carbamate; urea; thiocarbamate; thiourea; ketne; sulfone; sulfonamide; aryl including phenyl, naphthyl, and anthracenyl; heteroaryl including 5-member heteroaryls including as pyrrole, imidazole, furan, thiophene, oxazole, thiazole, isoxazole, isothiazole, thiadiazole, triazole, oxadiazole, and tetrazole, 6-member heteroaryls including pyridine, pyrimidine, pyrazine, and fused heteroaryls including benzofuran, benzothiophene, benzoxazole, benzimidazole, indole, benzothiazole, benzisoxazole, and benzisothiazole.

In certain embodiments -L¹- of formula (V) is substituted with one moiety -L²-Z.

Another embodiment for -L¹- is disclosed in U.S. Pat. No. 7,585,837B2, which is herewith incorporated by reference in its entirety. Accordingly, in certain embodiments -L¹- is of formula (VI):

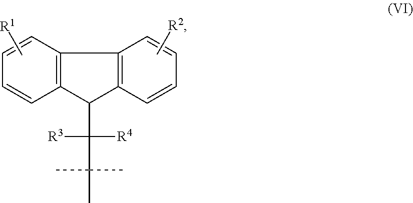

wherein
the dashed line indicates attachment to -D which is a PTH moiety and wherein attachment is through an amine functional group of -D;
R¹ and R² are independently selected from the group consisting of hydrogen, alkyl, alkoxy, alkoxyalkyl, aryl, alkaryl, aralkyl, halogen, nitro, —SO₃H, —SO₂NHR⁵, amino, ammonium, carboxyl, PO₃H₂, and OPO₃H₂;
R³, R⁴, and R⁵ are independently selected from the group consisting of hydrogen, alkyl, and aryl;
wherein -L¹- is substituted with -L²-Z and wherein -L¹- is optionally further substituted;
   wherein
   -L²- is a single chemical bond or a spacer; and
   —Z is a water-soluble carrier.

Suitable substituents for formulas (VI) are alkyl (such as C₁₋₆ alkyl), alkenyl (such as C₂₋₆ alkenyl), alkynyl (such as C₂₋₆ alkynyl), aryl (such as phenyl), heteroalkyl, heteroalkenyl, heteroalkynyl, heteroaryl (such as aromatic 4 to 7 membered heterocycle) or halogen moieties.

Only in the context of formula (VI) the terms used have the following meaning:

The terms “alkyl”, “alkoxy”, “alkoxyalkyl”, “aryl”, “alkaryl” and “aralkyl” mean alkyl radicals of 1-8, preferably 1-4 carbon atoms, e.g. methyl, ethyl, propyl, isopropyl and butyl, and aryl radicals of 6-10 carbon atoms, e.g. phenyl and naphthyl. The term “halogen” includes bromo, fluoro, chloro and iodo.

In certain embodiments -L¹- of formula (VI) is substituted with one moiety -L²-Z.

Another embodiment for -L¹- is disclosed in WO2002/089789A1, which is herewith incorporated by reference in its entirety. Accordingly, in certain embodiments -L¹- is of formula (VII):

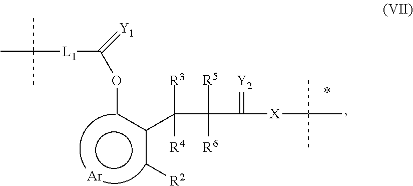

wherein
the dashed line indicates attachment to -D which is a PTH moiety and wherein attachment is through an amine functional group of -D;
L₁ is a bifunctional linking group,
Y₁ and Y₂ are independently O, S or NR⁷;
R², R³, R⁴, R⁵, R⁶ and R⁷ are independently selected from the group consisting of hydrogen, C₁₋₆ alkyls, C₃₋₁₂ branched alkyls, C₃₋₈ cycloalkyls, C₁₋₆ substituted alkyls, C₃₋₈ substituted cycloalkyls, aryls, substituted aryls, aralkyls, C₁₋₆ heteroalkyls, substituted C₁₋₆ heteroalkyls, C₁₋₆ alkoxy, phenoxy, and C₁₋₆ heteroalkoxy;
Ar is a moiety which when included in formula (VII) forms a multisubstituted aromatic hydrocarbon or a multi-substituted heterocyclic group;
X is a chemical bond or a moiety that is actively transported into a target cell, a hydrophobic moiety, or a combination thereof,
y is 0 or 1;
wherein -L¹- is substituted with -L²-Z and wherein -L¹- is optionally further substituted;
   wherein
   -L²- is a single chemical bond or a spacer; and
   —Z is a water-soluble carrier.

Only in the context of formula (VII) the terms used have the following meaning:

The term “alkyl” shall be understood to include, e.g. straight, branched, substituted C₁₋₁₂ alkyls, including alkoxy, C₃₋₈ cycloalkyls or substituted cycloalkyls, etc.

The term “substituted” shall be understood to include adding or replacing one or more atoms contained within a functional group or compounds with one or more different atoms.

Substituted alkyls include carboxyalkyls, aminoalkyls, dialkylaminos, hydroxyalkyls and mercaptoalkyls; substituted cycloalkyls include moieties such as 4-chlorocyclohexyl; aryls include moieties such as napthyl; substituted aryls include moieties such as 3-bromo-phenyl; aralkyls include moieties such as toluyl; heteroalkyls include moieties such as ethylthiophene; substituted heteroalkyls include moieties such as 3-methoxythiophone; alkoxy includes moeities such as methoxy; and phenoxy includes moieties such as 3-nitrophenoxy. Halo-shall be understood to include fluoro, chloro, iodo and bromo.

In certain embodiments -L¹- of formula (VII) is substituted with one moiety -L²-Z.

In certain embodiments -L¹- comprises a substructure of formula (VIII)

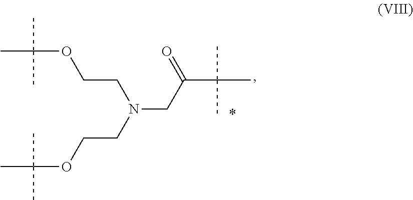

wherein
the dashed line marked with the asterisk indicates attachment to a nitrogen of -D which is a PTH moiety by forming an amide bond;
the unmarked dashed lines indicate attachment to the remainder of -L¹-; and
wherein -L¹- is substituted with -L²-Z and wherein -L¹- is optionally further substituted;
   wherein
   -L²- is a single chemical bond or a spacer; and
   —Z is a water-soluble carrier.

In certain embodiments -L¹- of formula (VIII) is substituted with one moiety -L²-Z.

In certain embodiments -L¹- of formula (VIII) is not further substituted.

In certain embodiments -L¹- comprises a substructure of formula (IX)

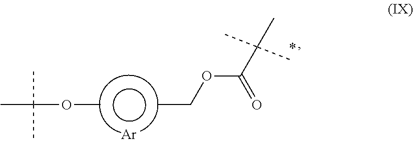

wherein
the dashed line marked with the asterisk indicates attachment to a nitrogen of -D which is a PTH moiety by forming a carbamate bond;
the unmarked dashed lines indicate attachment to the remainder of -L¹-; and
wherein -L¹- is substituted with -L²-Z and wherein -L¹- is optionally further substituted;
   wherein
   -L²- is a single chemical bond or a spacer; and
   —Z is a water-soluble carrier.

In certain embodiments -L¹- of formula (IX) is substituted with one moiety -L²-Z.

In certain embodiments -L¹- of formula (IX) is not further substituted.

In certain embodiments -L¹- has a structure as disclosed in WO2020/206358 A1.

Accordingly, in certain embodiments the moiety -L¹- is of formula (X):

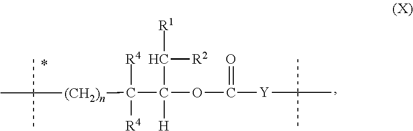

wherein
the unmarked dashed line indicates attachment to -D;
the dashed line marked with the asterisk indicates attachment to -L²-Z;
n is an integer selected from the group consisting of 0, 1, 2, 3, 4, 5 and 6;
—R¹ and —R² are independently an electron-withdrawing group, alkyl, or —H, and wherein at least one of —R¹ or —R² is an electron-withdrawing group;
each —R⁴ is independently C₁-C₃ alkyl or the two —R⁴ are taken together with the carbon atom to which they are attached to form a 3- to 6-membered ring; and —Y— is absent when -D is a drug moiety connected through an amine, or —Y— is —N(R⁶)CH₂— when -D is a drug moiety connected through a phenol, alcohol, thiol, thiophenol, imidazole, or non-basic amine; wherein —R⁶ is optionally substituted C₁-C₆ alkyl, optionally substituted aryl, or optionally substituted heteroaryl.

In certain embodiments n of formula (X) is an integer selected from 1, 2, 3, 4, 5 and 6. In certain embodiments n of formula (X) is an integer selected from 1, 2 and 3. In certain embodiments n of formula (X) is an integer from 0, 1, 2 and 3. In certain embodiments n of formula (X) is 1. In certain embodiments n of formula (X) is 2. In certain embodiments n of formula (X) is 3.

In certain embodiments the electron-withdrawing group of —R¹ and —R² of formula (X) is selected from the group consisting of —CN; —NO₂; optionally substituted aryl; optionally substituted heteroaryl; optionally substituted alkenyl; optionally substituted alkynyl; —COR³, —SOR³, or —SO₂R³, wherein —R³ is —H, optionally substituted alkyl, optionally substituted aryl, optionally substituted arylalkyl, optionally substituted heteroaryl, optionally substituted heteroarylalkyl, —OR⁸ or —NR⁸₂, wherein each —R⁸ is independently —H or optionally substituted alkyl, or both —R⁸ groups are taken together with the nitrogen to which they are attached to form a heterocyclic ring; or —SR⁹, wherein —R⁹ is optionally substituted alkyl, optionally substituted aryl, optionally substituted arylalkyl, optionally substituted heteroaryl, or optionally substituted heteroarylalkyl.

In certain embodiments the electron-withdrawing group of —R¹ and —R² of formula (X) is —CN. In certain embodiments the electron-withdrawing group of —R¹ and —R² of formula (X) is —NO₂. In certain embodiments the electron-withdrawing group of —R¹ and —R² of formula (X) is optionally substituted aryl comprising 6 to 10 carbons. In certain embodiments the electron-withdrawing group of —R¹ and —R² of formula (X) is optionally substituted phenyl, naphthyl, or anthracenyl. In certain embodiments the electron-withdrawing group of —R¹ and —R² of formula (X) is optionally substituted heteroaryl comprising 3 to 7 carbons and comprising at least one N, O, or S atom. In certain embodiments the electron-withdrawing group of —R¹ and —R² of formula (X) is optionally substituted pyrrolyl, pyridyl, pyrimidinyl, imidazolyl, oxazolyl, isoxazolyl, thiazolyl, isothiazolyl, quinolyl, indolyl, or indenyl. In certain embodiments the electron-withdrawing group of —R¹ and —R² of formula (X) is optionally substituted alkenyl containing 2 to 20 carbon atoms. In certain embodiments the electron-withdrawing group of —R¹ and —R² of formula (X) is optionally substituted alkynyl comprising 2 to 20 carbon atoms. In certain embodiments the electron-withdrawing group of —R¹ and —R² of formula (X) is —COR³, —SOR³, or —SO₂R³, wherein —R³ is —H, optionally substituted alkyl comprising 1 to 20 carbon atoms, optionally substituted aryl, optionally substituted arylalkyl, optionally substituted heteroaryl, optionally substituted heteroarylalkyl, —OR⁸ or —NR⁸², wherein each —R⁸ is independently —H or optionally substituted alkyl comprising 1 to 20 carbon atoms, or both —R⁸ groups are taken together with the nitrogen to which they are attached to form a heterocyclic ring. In certain embodiments the electron-withdrawing group of —R¹ and —R² of formula (X) is —SR⁹, wherein —R⁹ is optionally substituted alkyl comprising 1 to 20 carbon atoms, optionally substituted aryl, optionally substituted arylalkyl, optionally substituted heteroaryl, or optionally substituted heteroarylalkyl.

In certain embodiments at least one of —R¹ or —R² of formula (X) is —CN, —SOR³ or —SO₂R³. In certain embodiments at least one of —R¹ and —R² of formula (X) is —CN or —SO₂R³. In certain embodiments at least one of —R¹ and —R² of formula (X) is —CN or —SO₂R³, wherein —R³ is optionally substituted alkyl, optionally substituted aryl, or —NR⁸₂. In certain embodiments at least one of —R¹ and —R² of formula (X) is —CN, —SO₂N(CH₃)₂, —SO₂CH₃, phenyl substituted with —SO₂, phenyl substituted with —SO₂ and —Cl, —SO₂N(CH₂CH₂)₂₀, —SO₂CH(CH₃)₂, —SO₂N(CH₃)(CH₂CH₃), or —SO₂N(CH₂CH₂OCH₃)₂.

In certain embodiments each —R⁴ of formula (X) is independently C₁-C₃ alkyl. In certain embodiments both —R⁴ are methyl.

In certain embodiments —Y— of formula (X) is absent. In certain embodiments —Y— of formula (X) is —N(R⁶)CH₂—.

In certain embodiments -L¹- is of formula (X), wherein n is 1, —R¹ is —CN, —R² is —H, and —R⁴ is —CH₃. In certain embodiments -L¹- is of formula (X), wherein n is 1, —R¹ is —SO₂N(CH₃)₂, —R² is —H, and —R⁴ is —CH₃. In certain embodiments -L¹- is of formula (X), wherein n is 1, —R¹ is SO₂CH₃, —R² is —H, and —R⁴ is —CH₃. In certain embodiments -L¹- is of formula (X), wherein n is 1, —R¹ is —SO₂N(CH₂CH₂)₂CHCH₃, —R² is —H, and —R⁴ is —CH₃. In certain embodiments -L¹- is of formula (X), wherein n is 1, —R¹ is phenyl substituted with —SO₂, —R² is —H, and —R⁴ is —CH₃. In certain embodiments -L¹- is of formula (X), wherein n is 1, —R¹ is phenyl substituted with —SO₂ and —Cl, —R² is —H, and —R⁴ is —CH₃. In certain embodiments -L¹- is of formula (X), wherein n is 1, —R¹ is —SO₂N(CH₂CH₂)₂₀, —R² is —H, and —R⁴ is —CH₃. In certain embodiments -L¹- is of formula (X), wherein n is 1, —R¹ is —SO₂CH(CH₃)₂, —R² is —H, and —R⁴ is —CH₃. In certain embodiments -L¹- is of formula (X), wherein n is 1, —R¹ is —SO₂N(CH₃)(CH₂CH₃), —R² is —H, and —R⁴ is —CH₃. In certain embodiments -L¹- is of formula (X), wherein n is 1, —R¹ is —SO₂N(CH₂CH₂OCH₃)₂, —R² is —H, and —R⁴ is —CH₃. In certain embodiments -L¹- is of formula (X), wherein n is 1, —R¹ is phenyl substituted with —SO₂ and —CH₃, —R² is —H, and —R⁴ is —CH₃.

In certain embodiments -L¹- is of formula (X), wherein n is 2, —R¹ is —CN, —R² is —H, and —R⁴ is —CH₃. In certain embodiments -L¹- is of formula (X), wherein n is 2, —R¹ is —SO₂N(CH₃)₂, —R² is —H, and —R⁴ is —CH₃. In certain embodiments -L¹- is of formula (X), wherein n is 2, —R¹ is SO₂CH₃, —R² is —H, and —R⁴ is —CH₃. In certain embodiments -L¹- is of formula (X), wherein n is 2, —R¹ is —SO₂N(CH₂CH₂)₂CHCH₃, —R² is —H, and —R⁴ is —CH₃. In certain embodiments -L¹- is of formula (X), wherein n is 2, —R¹ is phenyl substituted with —SO₂, —R² is —H, and —R⁴ is —CH₃. In certain embodiments -L¹- is of formula (X), wherein n is 2, —R¹ is phenyl substituted with —SO₂ and —Cl, —R² is —H, and —R⁴ is —CH₃. In certain embodiments -L¹- is of formula (X), wherein n is 2, —R¹ is —SO₂N(CH₂CH₂)₂₀, —R² is —H, and —R⁴ is —CH₃. In certain embodiments -L¹- is of formula (X), wherein n is 2, —R¹ is —SO₂CH(CH₃)₂, —R² is —H, and —R⁴ is —CH₃. In certain embodiments -L¹- is of formula (X), wherein n is 2, —R¹ is —SO₂N(CH₃)(CH₂CH₃), —R² is —H, and —R⁴ is —CH₃. In certain embodiments -L¹- is of formula (X), wherein n is 2, —R¹ is —SO₂N(CH₂CH₂OCH₃)₂, —R² is —H, and —R⁴ is —CH₃. In certain embodiments -L¹- is of formula (X), wherein n is 2, —R¹ is phenyl substituted with —SO₂ and —CH₃, —R² is —H, and —R⁴ is —CH₃.

In certain embodiments -L¹- is of formula (X), wherein n is 3, —R¹ is —CN, —R² is —H, and —R⁴ is —CH₃. In certain embodiments -L¹- is of formula (X), wherein n is 3, —R¹ is —SO₂N(CH₃)₂, —R² is —H, and —R⁴ is —CH₃. In certain embodiments -L¹- is of formula (X), wherein n is 3, —R¹ is SO₂CH₃, —R² is —H, and —R⁴ is —CH₃. In certain embodiments -L¹- is of formula (X), wherein n is 3, —R¹ is —SO₂N(CH₂CH₂)₂CHCH₃, —R² is —H, and —R⁴ is —CH₃. In certain embodiments -L¹- is of formula (X), wherein n is 3, —R¹ is phenyl substituted with —SO₂, —R² is —H, and —R⁴ is —CH₃. In certain embodiments -L¹- is of formula (X), wherein n is 3, —R¹ is phenyl substituted with —SO₂ and —Cl, —R² is —H, and —R⁴ is —CH₃. In certain embodiments -L¹- is of formula (X), wherein n is 3, —R¹ is —SO₂N(CH₂CH₂)₂₀, —R² is —H, and —R⁴ is —CH₃. In certain embodiments -L¹- is of formula (X), wherein n is 3, —R¹ is —SO₂CH(CH₃)₂, —R² is —H, and —R⁴ is —CH₃. In certain embodiments -L¹- is of formula (X), wherein n is 3, —R¹ is —SO₂N(CH₃)(CH₂CH₃), —R² is —H, and —R⁴ is —CH₃. In certain embodiments -L¹- is of formula (X), wherein n is 3, —R¹ is —SO₂N(CH₂CH₂OCH₃)₂, —R² is —H, and —R⁴ is —CH₃. In certain embodiments -L¹- is of formula (X), wherein n is 3, —R¹ is phenyl substituted with —SO₂ and —CH₃, —R² is —H, and —R⁴ is —CH₃.

Only in the context of formula (X) the terms used have the following meaning:

The term “alkyl” refers to linear, branched, or cyclic saturated hydrocarbon groups of 1 to 20, 1 to 12, 1 to 8, 1 to 6, or 1 to 4 carbon atoms. In certain embodiments an alkyl is linear or branched. Examples of linear or branched alkyl groups include methyl, ethyl, n-propyl, isopropyl, n-butyl, t-butyl, isobutyl, sec-butyl, n-pentyl, n-hexyl, n-heptyl, n-octyl, n-nonyl, and n-decyl. In certain embodiments an alkyl is cyclic. Examples of cyclic alkyl groups include cyclopropyl, cyclobutyl, cyclopentyl, cyclopentadienyl, and cyclohexyl.

The term “alkoxy” refers to alkyl groups bonded to oxygen, including methoxy, ethoxy, isopropoxy, cyclopropoxy, and cyclobutoxy.

The term “alkenyl” refers to non-aromatic unsaturated hydrocarbons with carbon-carbon double bonds and 2 to 20, 2 to 12, 2 to 8, 2 to 6, or 2 to 4 carbon atoms.

The term “alkynyl” refers to non-aromatic unsaturated hydrocarbons with carbon-carbon triple bonds and 2 to 20, 2 to 12, 2 to 8, 2 to 6, or 2 to 4 carbon atoms.

The term “aryl” refers to aromatic hydrocarbon groups of 6 to 18 carbons, preferably 6 to 10 carbons, including groups such as phenyl, naphthyl, and anthracenyl. The term “heteroaryl” refers to aromatic rings comprising 3 to 15 carbons comprising at least one N, O or S atom, preferably 3 to 7 carbons comprising at least one N, O or S atom, including groups such as pyrrolyl, pyridyl, pyrimidinyl, imidazolyl, oxazolyl, isoxazolyl, thiazolyl, isothiazolyl, quinolyl, indolyl, and indenyl.

In certain embodiments alkenyl, alkynyl, aryl or heteroaryl moieties may be coupled to the remainder of the molecule through an alkyl linkage. Under those circumstances, the substituent will be referred to as alkenylalkyl, alkynylalkyl, arylalkyl or heteroarylalkyl, indicating that an alkylene moiety is between the alkenyl, alkynyl, aryl or heteroaryl moiety and the molecule to which the alkenyl, alkynyl, aryl or heteroaryl is coupled.

The term “halogen” or “halo” refers to bromo, fluoro, chloro and iodo.

The term “heterocyclic ring” or “heterocyclyl” refers to a 3- to 15-membered aromatic or non-aromatic ring comprising at least one N, O, or S atom. Examples include piperidinyl, piperazinyl, tetrahydropyranyl, pyrrolidine, and tetrahydrofuranyl, as well as the exemplary groups provided for the term “heteroaryl” above. In certain embodiments a heterocyclic ring or heterocyclyl is non-aromatic. In certain embodiments a heterocyclic ring or heterocyclyl is aromatic.

The term “optionally substituted” refers to a group may be unsubstituted or substituted by one or more (e.g., 1, 2, 3, 4 or 5) of the substituents which may be the same or different. Examples of substituents include alkyl, alkenyl, alkynyl, halogen, —CN, —OR^{aa}, —SR^{aa}, —NR^{aa}R^{bb}, —NO₂, —C═NH(OR^{aa}), —C(O)R^{aa}, —OC(O)R^{aa}, —C(O)OR^{aa}, —C(O)NR^{aa}R^{bb}, —OC(O)NR^{aa}R^{bb}, —NR^{aa}C(O)R^{bb}, —NR^{aa}C(O)OR^{bb}, —S(O)R^{aa}, —S(O)₂R^{aa}, —NR^{aa}S(O)R^{bb}, —C(O)NR^{aa}S(O)R^{bb}, —NR^{aa}S(O)₂R^{bb}, —C(O)NR^{aa}S(O)₂R^{bb}, —S(O)NR^{aa}R^{bb}, —S(O)₂NR^{aa}R^{bb}, —P(O)(OR^{aa})(OR^{bb}), heterocyclyl, heteroaryl, or aryl, wherein the alkyl, alkenyl, alkynyl, cycloalkyl, heterocyclyl, heteroaryl, and aryl are each independently optionally substituted by —R^{cc}, wherein —R^{aa} and —R^{bb} are each independently —H, alkyl, alkenyl, alkynyl, heterocyclyl, heteroaryl, or aryl, or —R^{aa} and —R^{bb} are taken together with the nitrogen atom to which they attach to form a heterocyclyl, which is optionally substituted by alkyl, alkenyl, alkynyl, halogen, hydroxyl, alkoxy, or —CN, and wherein: each —R^{cc} is independently alkyl, alkenyl, alkynyl, halogen, heterocyclyl, heteroaryl, aryl, —CN, or —NO₂.

-L²- is a chemical bond or a spacer moiety.

In certain embodiments -L²- is a chemical bond.

In certain embodiments -L²- is a spacer moiety, such as a spacer moiety selected from the group consisting of -T-, —C(O)O—, —O—, —C(O)—, —C(O)N(R^{y1})—, —S(O)₂N(R^{y1})—, —S(O)N(R^{y1})—, —S(O)₂—, —S(O)—, —N(R^{y1})S(O)₂N(R^{y1a})—, —S—, —N(R^{y1})—, —OC(OR^{y1})(R^{y1a})—, —N(R^{y1})C(O)N(R^{y1a})—, —OC(O)N(R^{y1})—, C₁₋₅₀ alkyl, C₂₋₅₀ alkenyl, and C₂₋₅₀ alkynyl; wherein -T-, C₁₋₅₀ alkyl, C₂₋₅₀ alkenyl, and C₂₋₅₀ alkynyl are optionally substituted with one or more —R^{y2}, which are the same or different and wherein C₁₋₅₀ alkyl, C₂₋₅₀ alkenyl, and C₂₋₅₀ alkynyl are optionally interrupted by one or more groups selected from the group consisting of -T-, —C(O)O—, —O—, —C(O)—, —C(O)N(R^{y3})—, —S(O)₂N(R^{y3})—, —S(O)N(R^{y3})—, —S(O)₂—, —S(O)—, —N(R^{y3})S(O)₂N(R^{y3a})—, —S—, —N(R^{y3})—, —OC(OR^{y3})(R^{y3a})—, —N(R^{y3})C(O)N(R^{y3a})—, and —OC(O)N(R^{y3})—;

—R^{y1} and —R^{y1a} are independently of each other selected from the group consisting of —H, -T, C₁₋₅₀ alkyl, C₂₋₅₀ alkenyl, and C₂₋₅₀ alkynyl; wherein -T, C₁₋₅₀ alkyl, C₂₋₅₀ alkenyl, and C₂₋₅₀ alkynyl are optionally substituted with one or more —R^{y2}, which are the same or different, and wherein C₁₋₅₀ alkyl, C₂₋₅₀ alkenyl, and C₂₋₅₀ alkynyl are optionally interrupted by one or more groups selected from the group consisting of -T-, —C(O)O—, —O—, —C(O)—, —C(O)N(R^{y4})—, —S(O)₂N(R^{y4})—, —S(O)N(R^{y4})—, —S(O)₂—, —S(O)—, —N(R^{y4})S(O)₂N(R^{y4a})—, —S—, —N(R^{y4})—, —OC(OR^{y4})(R^{y4a})—, —N(R^{y4})C(O)N(R^{y4a})—, and —OC(O)N(R^{y4})—;

each T is independently selected from the group consisting of phenyl, naphthyl, indenyl, indanyl, tetralinyl, C₃₋₁₀ cycloalkyl, 3- to 10-membered heterocyclyl, 8- to 11-membered heterobicyclyl, 8- to 30-membered carbopolycyclyl, and 8- to 30-membered heteropolycyclyl; wherein each T is independently optionally substituted with one or more —R^{y2}, which are the same or different;

each —R^{y2} is independently selected from the group consisting of halogen, —CN, oxo (═O), —COOR^{y5}, —OR^{y5}, —C(O)R^{y5}, —C(O)N(R^{y5}R^{y5a}), —S(O)₂N(R^{y5}R^{y5a}), —S(O)N(R^{y5}R^{y5a}), —S(O)₂R^{y5}, —S(O)R^{y5}, —N(R^{y5})S(O)₂N(R^{y5a}R^{y5}b), —SR^{y5}, —N(R^{y5}R^{y5a}), —NO₂, —OC(O)R^{y5}, —N(R^{y5})C(O)R^{y5a}, —N(R^{y5})S(O)₂R^{y5a}, —N(R^{y5})S(O)R^{y5a}, —N(R^{y5})C(O)OR^{y5a}, —N(R^{y5})C(O)N(R^{y5a}R^{y5b}), —OC(O)N(R^{y5}R^{y5a}), and C₁₋₆ alkyl; wherein C₁₋₆ alkyl is optionally substituted with one or more halogen, which are the same or different; and

each —R^{y3}, —R^{y3a}, —R^{y4}, —R^{y4a}, —R^{y5}, —R^{y5a} and —R^{y5b} is independently selected from the group consisting of —H, and C₁₋₆ alkyl, wherein C₁₋₆ alkyl is optionally substituted with one or more halogen, which are the same or different.

In certain embodiments -L²- selected from -T-, —C(O)O—, —O—, —C(O)—, —C(O)N(R^{y1})—, —S(O)₂N(R^{y1})—, —S(O)N(R^{y1})—, —S(O)₂—, —S(O)—, —N(R^{y1})S(O)₂N(R^{y1a})—, —S—, —N(R^{y1})—, —OC(OR^{y1})(R^{y1a})—, —N(R^{y1})C(O)N(R^{y1a})—, —OC(O)N(R^{y1})—, C₁₋₅₀ alkyl, C₂₋₅₀ alkenyl, and C₂₋₅₀ alkynyl; wherein -T-, C₁₋₂₀ alkyl, C₂₋₂₀ alkenyl, and C₂₋₂₀ alkynyl are optionally substituted with one or more —R^{y2}, which are the same or different and wherein C₁₋₂₀ alkyl, C₂₋₂₀ alkenyl, and C₂₋₂₀ alkynyl are optionally interrupted by one or more groups selected from the group consisting of -T-, —C(O)O—, —O—, —C(O)—, —C(O)N(R^{y3})—, —S(O)₂N(R^{y3})—, —S(O)N(R^{y3})—, —S(O)₂—, —S(O)—, —N(R^{y3})S(O)₂N(R^{y3a})—, —S—, —N(R^{y3})—, —OC(OR^{y3})(R^{y3a})—, —N(R^{y3})C(O)N(R^{y3a})—, and —OC(O)N(R^{y3})—;

—R^{y1} and —R^{y1a} are independently of each other selected from the group consisting of —H, -T, C₁₋₁₀ alkyl, C₂₋₁₀ alkenyl, and C₂₋₁₀ alkynyl; wherein -T, C₁₋₁₀ alkyl, C₂₋₁₀ alkenyl, and C₂₋₁₀ alkynyl are optionally substituted with one or more —R^{y2}, which are the same or different, and wherein C₁₋₁₀ alkyl, C₂₋₁₀ alkenyl, and C₂₋₁₀ alkynyl are optionally interrupted by one or more groups selected from the group consisting of -T-, —C(O)O—, —O—, —C(O)—, —C(O)N(R^{y4})—, —S(O)₂N(R^{y4})—, —S(O)N(R^{y4})—, —S(O)₂—, —S(O)—, —N(R^{y4})S(O)₂N(R^{y4a})—, —S—, —N(R^{y4})—, —OC(OR^{y4})(R^{y4a})—, —N(R^{y4})C(O)N(R^{y4a})—, and —OC(O)N(R^{y4})—;

each T is independently selected from the group consisting of phenyl, naphthyl, indenyl, indanyl, tetralinyl, C₃₋₁₀ cycloalkyl, 3- to 10-membered heterocyclyl, 8- to 11-membered heterobicyclyl, 8- to 30-membered carbopolycyclyl, and 8- to 30-membered heteropolycyclyl; wherein each T is independently optionally substituted with one or more —R^{y2}, which are the same or different; —R^{y2} is selected from the group consisting of halogen, —CN, oxo (═O), —COOR^{y5}, —OR^{y5}, —C(O)R^{y5}, —C(O)N(R^{y5}R^{y5a}), —S(O)₂N(R^{y5}R^{y5a}), —S(O)N(R^{y5}R^{y5a}), —S(O)₂R^{y5}, —S(O)R^{y5}, —N(R^{y5})S(O)₂N(R^{y5a}R^{y5}b), —SR^{y5}, —N(R^{y5}R^{y5a}), —NO₂, —OC(O)R^{y5}, —N(R^{y5})C(O)R^{y5a}, —N(R^{y5})S(O)₂R^{y5a}, —N(R^{y5})S(O)R^{y5a}, —N(R^{y5})C(O)OR^{y5a}, —N(R^{y5})C(O)N(R^{y5a}R^{y5}b), —OC(O)N(R^{y5}R^{y5a}), and C₁₋₆ alkyl; wherein C₁₋₆ alkyl is optionally substituted with one or more halogen, which are the same or different; and

each —R^{y3}, —R^{y3a}, —R^{y4}, —R^{y4a}, —R^{y5}, —R^{y5a} and —R^{y5b} is independently of each other selected from the group consisting of —H, and C₁₋₆ alkyl; wherein C₁₋₆ alkyl is optionally substituted with one or more halogen, which are the same or different.

In certain embodiments -L²- is selected from the group consisting of -T-, —C(O)O—, —O—, —C(O)—, —C(O)N(R^{y1})—, —S(O)₂N(R^{y1})—, —S(O)N(R^{y1})—, —S(O)₂—, —S(O)—, —N(R^{y1})S(O)₂N(R^{y1a})—, —S—, —N(R^{y1})—, —OC(OR^{y1})(R^{y1a})—, —N(R^{y1})C(O)N(R^{y1a})—, —OC(O)N(R^{y1})—, C₁₋₅₀ alkyl, C₂₋₅₀ alkenyl, and C₂₋₅₀ alkynyl; wherein -T-, C₁₋₅₀ alkyl, C₂₋₅₀ alkenyl, and C₂₋₅₀ alkynyl are optionally substituted with one or more —R^{y2}, which are the same or different and wherein C₁₋₅₀ alkyl, C₂₋₅₀ alkenyl, and C₂₋₅₀ alkynyl are optionally interrupted by one or more groups selected from the group consisting of -T-, —C(O)O—, —O—, —C(O)—, —C(O)N(R^{y3})—, —S(O)₂N(R^{y3})—, —S(O)N(R^{y3})—, —S(O)₂—, —S(O)—, —N(R^{y3})S(O)₂N(R^{y3a})—, —S—, —N(R^{y3})—, —OC(OR^{y3})(R^{y3a})—, —N(R^{y3})C(O)N(R^{y3a})—, and —OC(O)N(R^{y3})—;

—R^{y1} and —R^{y1a} are independently selected from the group consisting of —H, -T, C₁₋₁₀ alkyl, C₂₋₁₀ alkenyl, and C₂₋₁₀ alkynyl;

each T is independently selected from the group consisting of phenyl, naphthyl, indenyl, indanyl, tetralinyl, C₃₋₁₀ cycloalkyl, 3- to 10-membered heterocyclyl, 8- to 11-membered heterobicyclyl, 8- to 30-membered carbopolycyclyl, and 8- to 30-membered heteropolycyclyl;

each —R^{y2} is independently selected from the group consisting of halogen, and C₁₋₆ alkyl; and

each —R^{y3}, —R^{y3a}, —R^{y4}, —R^{y4a}, —R^{y5}, —R^{y5a} and —R^{y5b} is independently of each other selected from the group consisting of —H, and C₁₋₆ alkyl; wherein C₁₋₆ alkyl is optionally substituted with one or more halogen, which are the same or different.

In certain embodiments -L²- is a C₁₋₂₀ alkyl chain, which is optionally interrupted by one or more groups independently selected from —O—, -T- and —C(O)N(R^{y1})—; and which C₁₋₂₀ alkyl chain is optionally substituted with one or more groups independently selected from —OH, -T and —C(O)N(R^{y6}R^{y6a}); wherein —R^{y1}, —R^{y6}, —R^{y6a} are independently selected from the group consisting of —H and C₁₋₄ alkyl and wherein T is selected from the group consisting of phenyl, naphthyl, indenyl, indanyl, tetralinyl, C₃₋₁₀ cycloalkyl, 3- to 10-membered heterocyclyl, 8- to 11-membered heterobicyclyl, 8- to 30-membered carbopolycyclyl, and 8- to 30-membered heteropolycyclyl.

In certain embodiments -L²- has a molecular weight in the range of from 14 g/mol to 750 g/mol.

In certain embodiments -L²- comprises a moiety selected from

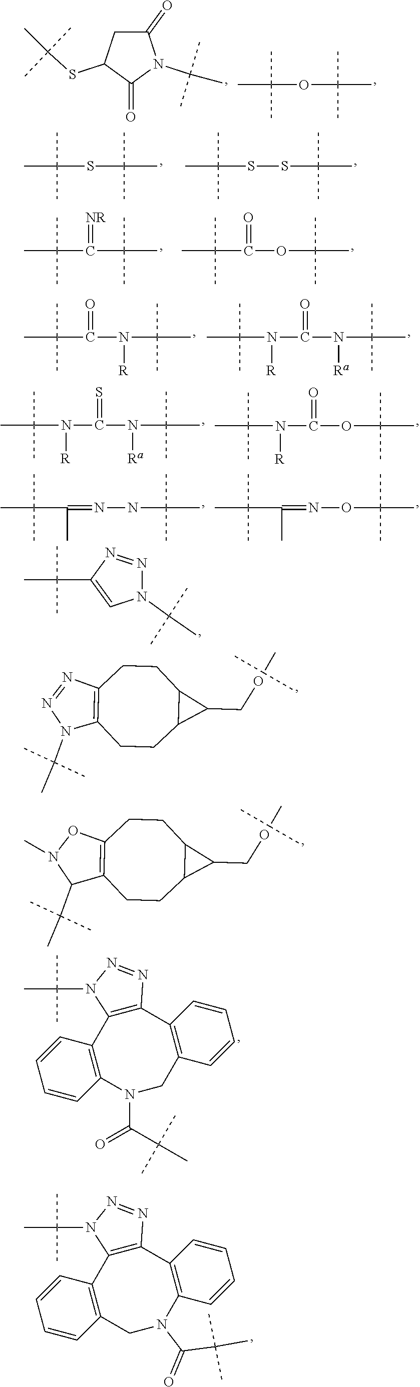

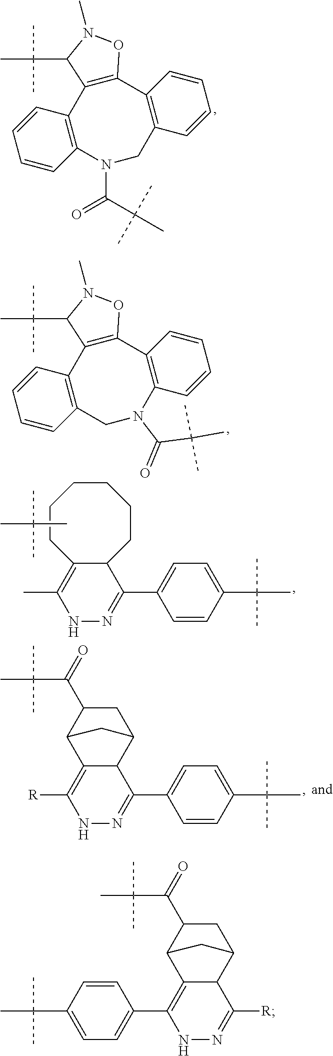

wherein
dashed lines indicate attachment to the rest of -L²-, -L¹- and/or —Z, respectively; and —R and —R^{a} are independently of each other selected from the group consisting of —H, methyl, ethyl, n-propyl, isopropyl, n-butyl, isobutyl, sec-butyl, tert-butyl, n-pentyl, 2-methylbutyl, 2,2-dimethylpropyl, n-hexyl, 2-methylpentyl, 3-methylpentyl, 2,2-dimethylbutyl, 2,3-dimethylbutyl and 3,3-dimethylpropyl.

In certain embodiments -L²- has a chain length of 1 to 20 atoms.

As used herein the term “chain length” with regard to the moiety -L²- refers to the number of atoms of -L²- present in the shortest connection between -L¹- and —Z.

In certain embodiments -L²- is of formula (i)

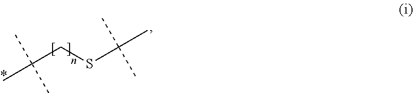

wherein
the dashed line marked with the asterisk indicates attachment to -L¹-;
the unmarked dashed line indicates attachment to —Z;
n is selected from the group consisting of 0, 1, 2, 3, 4, 5, 6, 7, 8, 9, 10, 11, 12, 13, 14, 15, 16, 17 and 18; and
wherein the moiety of formula (i) is optionally further substituted.

In certain embodiments n of formula (i) is selected from the group consisting of 3, 4, 5, 6, 7, 8, and 9. In certain embodiments n of formula (i) is 4, 5, 6, or 7. In certain embodiments n of formula (i) is 4. In certain embodiments n of formula (i) is 5. In certain embodiments n of formula (i) is 6.

In certain embodiments the moiety -L¹-L²- is selected from the group consisting of

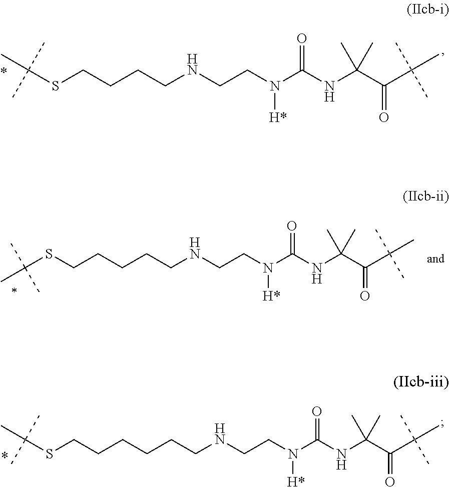

wherein
the unmarked dashed line indicates the attachment to a nitrogen of -D which is a PTH moiety by forming an amide bond; and
the dashed line marked with the asterisk indicates attachment to —Z.

In certain embodiments the moiety -L¹-L²- is of formula (IIca-i). In certain embodiments the moiety -L¹-L²- is of formula (IIca-ii). In certain embodiments the moiety -L¹-L²- is of formula (IIcb-iii).

In certain embodiments the PTH compound is of formula (Ia) with x=1. —Z comprises a C₈₋₂₄ alkyl or a polymer moiety. In certain embodiments —Z comprises a polymer moiety, such as a polymer selected from the group consisting of 2-methacryloyl-oxyethyl phosphoyl cholins, poly(acrylic acids), poly(acrylates), poly(acrylamides), poly(alkyloxy) polymers, poly(amides), poly(amidoamines), poly(amino acids), poly(anhydrides), poly(aspartamides), poly(butyric acids), poly(glycolic acids), polybutylene terephthalates, poly(caprolactones), poly(carbonates), poly(cyanoacrylates), poly(dimethylacrylamides), poly(esters), poly(ethylenes), poly(ethyleneglycols), poly(ethylene oxides), poly(ethyl phosphates), poly(ethyloxazolines), poly(glycolic acids), poly(hydroxyethyl acrylates), poly(hydroxyethyl-oxazolines), poly(hydroxymethacrylates), poly(hydroxypropylmethacrylamides), poly(hydroxypropyl methacrylates), poly(hydroxypropyloxazolines), poly(iminocarbonates), poly(lactic acids), poly(lactic-co-glycolic acids), poly(methacrylamides), poly(methacrylates), poly(methyloxazolines), poly(organophosphazenes), poly(ortho esters), poly(oxazolines), poly(propylene glycols), poly(siloxanes), poly(urethanes), poly(vinyl alcohols), poly(vinyl amines), poly(vinylmethylethers), poly(vinylpyrrolidones), silicones, celluloses, carbomethyl celluloses, hydroxypropyl methylcelluloses, chitins, chitosans, dextrans, dextrins, gelatins, hyaluronic acids and derivatives, functionalized hyaluronic acids, mannans, pectins, rhamnogalacturonans, starches, hydroxyalkyl starches, hydroxyethyl starches and other carbohydrate-based polymers, xylans, and copolymers thereof.

In certain embodiments —Z has a molecular weight ranging from 5 to 200 kDa. In certain embodiments —Z has a molecular weight ranging from 8 to 100 kDa, such as from 10 to 80 kDa, such as from 12 to 60 kDa, such as from 15 to 40 kDa. In certain embodiments —Z has a molecular weight of about 20 kDa. In certain embodiments —Z has a molecular weight of about 40 kDa.

In certain embodiments —Z comprises a protein. Preferred proteins are selected from the group consisting of carboxyl-terminal polypeptide of the chorionic gonadotropin as described in US 2012/0035101 A1 which are herewith incorporated by reference; albumin; XTEN sequences as described in WO 2011123813 A2, which are herewith incorporated by reference; proline/alanine random coil sequences as described in WO 2011/144756 A1 which are herewith incorporated by reference; proline/alanine/serine random coil sequences as described in WO 2008/155134 A1 and WO 2013/024049 A1 which are herewith incorporated by reference; and Fc fusion proteins.

In one embodiment —Z is a polysarcosine. In another preferred embodiment —Z comprises a poly(N-methylglycine). In a particularly preferred embodiment —Z comprises a random coil protein moiety. In one preferred embodiment —Z comprises at least one random coil protein moiety.

In certain embodiments —Z comprises a fatty acid derivate, such as a fatty acid derivative as disclosed in WO 2005/027978 A2 and WO 2014/060512 A1, which are herewith incorporated by reference.

In certain embodiments —Z is a hyaluronic acid-based polymer.

In certain embodiments —Z is a carrier as disclosed in WO 2012/02047 A1, which is herewith incorporated by reference.

In certain embodiments —Z is a carrier as disclosed in WO 2013/024048 A1, which is herewith incorporated by reference.

In certain embodiments —Z is a PEG-based polymer, such as a linear, branched or multi-arm PEG-based polymer. In certain embodiments —Z is a linear PEG-based polymer. In certain embodiments —Z is a multi-arm PEG-based polymer. In certain embodiments —Z is a multi-arm PEG-based polymer having at least 4 PEG-based arms.

In certain embodiments such multi-arm PEG-based polymer —Z is connected to a multitude of moieties -L²-L¹-D, wherein each moiety -L²-L¹-D is in certain embodiments connected to the end of an arm, in certain embodiments to the end of an arm. In certain embodiments such multi-arm PEG-based polymer —Z is connected to 2, 3, 4, 5, 6, 7, 8, 9, 10, 11, 12, 13, 14, 15 or 16 moieties -L²-L¹-D. In certain embodiments such multi-arm PEG-based polymer —Z is connected to 2, 3, 4, 6 or 8 moieties -L²-L¹-D. In certain embodiments such multi-arm PEG-based polymer —Z is connected to 2, 4 or 6 moieties -L²-L¹-D, in certain embodiments such multi-arm PEG-based polymer —Z is connected to 4 or 6 moieties -L²-L¹-D, and in certain embodiments such multi-arm PEG-based polymer —Z is connected to 4 moieties -L²-L¹-D.

In certain embodiments such multi-arm PEG-based polymer —Z is a multi-arm PEG derivative as, for instance, detailed in the products list of JenKem Technology, USA (accessed by download from http://www.jenkemusa.com/Pages/PEGProducts.aspx on Dec. 18, 2014), such as a 4-arm-PEG derivative, in particular a 4-arm-PEG comprising a pentaerythritol core, an 8-arm-PEG derivative comprising a hexaglycerin core, and an 8-arm-PEG derivative comprising a tripentaerythritol core. In certain embodiments the water-soluble PEG-based carrier —Z comprises a moiety selected from:

a 4-arm PEG Amine comprising a pentaerythritol core:

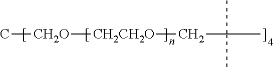

with n ranging from 20 to 500;

an 8-arm PEG Amine comprising a hexaglycerin core:

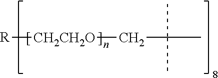

with n ranging from 20 to 500; and

R=hexaglycerin or tripentaerythritol core structure; and

a 6-arm PEG Amine comprising a sorbitol or dipentaerythritol core:

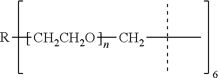

with n ranging from 20 to 500; and

R=comprising a sorbitol or dipentaerythritol core;

and wherein dashed lines indicate attachment to the rest of the PTH compound.

In certain embodiments —Z is a branched PEG-based polymer. In certain embodiments —Z is a branched PEG-based polymer having one, two, three, four, five or six branching points. In certain embodiments —Z is a branched PEG-based polymer having one, two or three branching points. In certain embodiments —Z is a branched PEG-based polymer having one branching point. In certain embodiments —Z is a branched PEG-based polymer having two branching points. In certain embodiments —Z is a branched PEG-based polymer having three branching points.

In certain embodiments a branching point is selected from the group consisting of —N<, —CH< and >C<.

In certain embodiments such branched PEG-based moiety —Z has a molecular weight of at least 10 kDa.

In certain embodiments such branched moiety —Z has a molecular weight ranging from and including 10 kDa to 500 kDa, such as from and including 10 kDa to 250 Da, such as ranging from and including 10 kDa to 150 kDa, such as from and including 12 kDa to 100 kDa and such as ranging from and including 15 kDa to 80 kDa.

In certain embodiments such branched moiety —Z has a molecular weight ranging from and including 10 kDa to 80 kDa. In certain embodiments the molecular weight is about 10 kDa. In certain embodiments the molecular weight of such branched moiety —Z is about 20 kDa. In certain embodiments the molecular weight of such branched moiety —Z is about 30 kDa. In certain embodiments the molecular weight of such a branched moiety —Z is about 40 kDa. In certain embodiments the molecular weight of such a branched moiety —Z is about 50 kDa. In certain embodiments the molecular weight of such a branched moiety —Z is about 60 kDa. In certain embodiments the molecular weight of such a branched moiety —Z is about 70 kDa. In certain embodiments the molecular weight of such a branched moiety —Z is about 80 kDa. In certain embodiments such branched moiety —Z has a molecular weight of about 40 kDa.

In certain embodiments —Z comprises a moiety

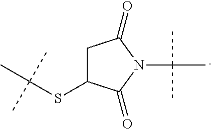

In certain embodiments —Z comprises an amide bond.

In certain embodiments —Z comprises a moiety of formula (a)

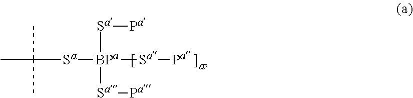

wherein
the dashed line indicates attachment to -L²- or to the remainder of —Z; BP^{a} is a branching point selected from the group consisting of —N<, —CR< and >C<; —R is selected from the group consisting of —H and C₁₋₆ alkyl;
a is 0 if BP^{a} is —N< or —CR< and n is 1 if BP^{a} is >C<;
—S^{a}—, —S^{a′}—, —S^{a″}— and —S^{a′″}— are independently of each other a chemical bond or are selected from the group consisting of C₁₋₅₀ alkyl, C₂₋₅₀ alkenyl, and C₂₋₅₀ alkynyl; wherein C₁₋₅₀ alkyl, C₂₋₅₀ alkenyl, and C₂₋₅₀ alkynyl are optionally substituted with one or more —R¹, which are the same or different and wherein C₁₋₅₀ alkyl, C₂₋₅₀ alkenyl, and C₂₋₅₀ alkynyl are optionally interrupted by one or more groups selected from the group consisting of -T-, —C(O)O—, —O—, —C(O)—, —C(O)N(R²)—, —S(O)₂N(R²)—, —S(O)N(R²)—, —S(O)₂—, —S(O)—, —N(R²)S(O)₂N(R^{2a})—, —S—, —N(R²)—, —OC(OR²)(R^{2a})—, —N(R²)C(O)N(R^{2a})—, and —OC(O)N(R²)—;
each -T- is independently selected from the group consisting of phenyl, naphthyl, indenyl, indanyl, tetralinyl, C₃₋₁₀ cycloalkyl, 3- to 10-membered heterocyclyl, 8- to 11-membered heterobicyclyl, 8- to 30-membered carbopolycyclyl, and 8- to 30-membered heteropolycyclyl; wherein each -T- is independently optionally substituted with one or more —R¹, which are the same or different;
each —R¹ is independently selected from the group consisting of halogen, —CN, oxo (═O), —COOR³, —OR³, —C(O)R³, —C(O)N(R³R^{3a}), —S(O)₂N(R³R^{3a}), —S(O)N(R³R^{3a}), —S(O)₂R³, —S(O)R³, —N(R³)S(O)₂N(R^{3a}R^{3b}), —SR³, —N(R³R^{3a}), —NO₂, —OC(O)R³, —N(R³)C(O)R^{3a}, —N(R³)S(O)₂R^{3a}, —N(R³)S(O)R^{3a}, —N(R³)C(O)OR^{3a}, —N(R³)C(O)N(R^{3a}R^{3b}), —OC(O)N(R³R^{3a}), and C₁₋₆ alkyl; wherein C₁₋₆ alkyl is optionally substituted with one or more halogen, which are the same or different;
each —R², —R^{2a}, —R³, —R^{3a} and —R^{3b} is independently selected from the group consisting of —H, and C₁₋₆ alkyl, wherein C₁₋₆ alkyl is optionally substituted with one or more halogen, which are the same or different; and —P^{a′}, P^{a″} and —P^{a′″} are independently a polymeric moiety.

In certain embodiments BP^{a} of formula (a) is —N<.

In certain embodiments BP^{a} of formula (a) is >C<.

In certain embodiments BP^{a} of formula (a) is —CR<. In certain embodiments —R is —H. Accordingly, a of formula (a) is 0.

In certain embodiments —S^{a}— of formula (a) is a chemical bond.

In certain embodiments —S^{a}— of formula (a) is selected from the group consisting of C₁₋₁₀ alkyl, C₂₋₁₀ alkenyl and C₂₋₁₀ alkynyl, which C₁₋₁₀ alkyl, C₂₋₁₀ alkenyl and C₂₋₁₀ alkynyl are optionally interrupted by one or more chemical groups selected from the group consisting of -T-, —C(O)O—, —O—, —C(O)—, —C(O)N(R⁴)—, —S(O)₂N(R⁴)—, —S(O)N(R⁴)—, —S(O)₂—, —S(O)—, —N(R⁴)S(O)₂N(R^{4a})—, —S—, —N(R⁴)—, —OC(OR⁴)(R^{4a})—, —N(R⁴)C(O)N(R^{4a})—, and —OC(O)N(R⁴)—; wherein -T- is a 3- to 10-membered heterocyclyl; and —R⁴ and —R^{4a} are independently selected from the group consisting of —H, methyl, ethyl, propyl and butyl.

In certain embodiments —S^{a}— of formula (a) is selected from the group consisting of C₁₋₁₀ alkyl which is interrupted by one or more chemical groups selected from the group consisting of -T-, —C(O)N(R⁴)— and —O—.

In certain embodiments —S^{a′}— of formula (a) is a chemical bond.

In certain embodiments —S^{a′}— of formula (a) is selected from the group consisting of C₁₋₁₀ alkyl, C₂₋₁₀ alkenyl and C₂₋₁₀ alkynyl, which C₁₋₁₀ alkyl, C₂₋₁₀ alkenyl and C₂₋₁₀ alkynyl are optionally interrupted by one or more chemical groups selected from the group consisting of —C(O)O—, —O—, —C(O)—, —C(O)N(R⁴)—, —S(O)₂N(R⁴)—, —S(O)N(R⁴)—, —S(O)₂—, —S(O)—, —N(R⁴)S(O)₂N(R^{4a})—, —S—, —N(R⁴)—, —OC(OR⁴)(R^{4a})—, —N(R⁴)C(O)N(R^{4a})—, and —OC(O)N(R⁴)—; wherein —R⁴ and —R^{4a} are independently selected from the group consisting of —H, methyl, ethyl, propyl and butyl. Preferably —S^{a′}— of formula (a) is selected from the group consisting of methyl, ethyl, propyl, butyl, which are optionally interrupted by one or more chemical groups selected from the group consisting of —O—, —C(O)— and —C(O)N(R⁴)—.

In certain embodiments —S^{a″}— of formula (a) is a chemical bond.

In certain embodiments —S^{a″}— of formula (a) is selected from the group consisting of C₁₋₁₀ alkyl, C₂₋₁₀ alkenyl and C₂₋₁₀ alkynyl, which C₁₋₁₀ alkyl, C₂₋₁₀ alkenyl and C₂₋₁₀ alkynyl are optionally interrupted by one or more chemical groups selected from the group consisting of —C(O)O—, —O—, —C(O)—, —C(O)N(R⁴)—, —S(O)₂N(R⁴)—, —S(O)N(R⁴)—, —S(O)₂—, —S(O)—, —N(R⁴)S(O)₂N(R^{4a})—, —S—, —N(R⁴)—, —OC(OR⁴)(R^{4a})—, —N(R⁴)C(O)N(R^{4a})—, and —OC(O)N(R⁴)—; wherein —R⁴ and —R^{4a} are independently selected from the group consisting of —H, methyl, ethyl, propyl and butyl. In certain embodiments —S^{a}″— of formula (a) is selected from the group consisting of methyl, ethyl, propyl, butyl, which are optionally interrupted by one or more chemical groups selected from the group consisting of —O—, —C(O)— and —C(O)N(R⁴)—.

In certain embodiments —S^{a′″}— of formula (a) is a chemical bond.

In certain embodiments —S^{a′″}— of formula (a) is selected from the group consisting of C₁₋₁₀ alkyl, C₂₋₁₀ alkenyl and C₂₋₁₀ alkynyl, which C₁₋₁₀ alkyl, C₂₋₁₀ alkenyl and C₂₋₁₀ alkynyl are optionally interrupted by one or more chemical groups selected from the group consisting of —C(O)O—, —O—, —C(O)—, —C(O)N(R⁴)—, —S(O)₂N(R⁴)—, —S(O)N(R⁴)—, —S(O)₂—, —S(O)—, —N(R⁴)S(O)₂N(R^{4a})—, —S—, —N(R⁴)—, —OC(OR⁴)(R^{4a})—, —N(R⁴)C(O)N(R^{4a})—, and —OC(O)N(R⁴)—; wherein —R⁴ and —R^{4a} are independently selected from the group consisting of —H, methyl, ethyl, propyl and butyl. In certain embodiments —S^{a′″}— of formula (a) is selected from the group consisting of methyl, ethyl, propyl, butyl, which are optionally interrupted by one or more chemical groups selected from the group consisting of —O—, —C(O)— and —C(O)N(R⁴)—.

In certain embodiments —P^{a′}, P^{a″} and —P^{a′″} of formula (a) independently comprise a polymer selected from the group consisting of 2-methacryloyl-oxyethyl phosphoyl cholins, poly(acrylic acids), poly(acrylates), poly(acrylamides), poly(alkyloxy) polymers, poly(amides), poly(amidoamines), poly(amino acids), poly(anhydrides), poly(aspartamides), poly(butyric acids), poly(glycolic acids), polybutylene terephthalates, poly(caprolactones), poly(carbonates), poly(cyanoacrylates), poly(dimethylacrylamides), poly(esters), poly(ethylenes), poly(ethyleneglycols), poly(ethylene oxides), poly(ethyl phosphates), poly(ethyloxazolines), poly(glycolic acids), poly(hydroxyethyl acrylates), poly(hydroxyethyl-oxazolines), poly(hydroxymethacrylates), poly(hydroxypropylmethacrylamides), poly(hydroxypropyl methacrylates), poly(hydroxypropyloxazolines), poly(iminocarbonates), poly(lactic acids), poly(lactic-co-glycolic acids), poly(methacrylamides), poly(methacrylates), poly(methyloxazolines), poly(organophosphazenes), poly(ortho esters), poly(oxazolines), poly(propylene glycols), poly(siloxanes), poly(urethanes), poly(vinyl alcohols), poly(vinyl amines), poly(vinylmethylethers), poly(vinylpyrrolidones), silicones, celluloses, carbomethyl celluloses, hydroxypropyl methylcelluloses, chitins, chitosans, dextrans, dextrins, gelatins, hyaluronic acids and derivatives, functionalized hyaluronic acids, mannans, pectins, rhamnogalacturonans, starches, hydroxyalkyl starches, hydroxyethyl starches and other carbohydrate-based polymers, xylans, and copolymers thereof.

In certain embodiments —P^{a′}, —P^{a″} and —P^{a′″} of formula (a) independently comprise a PEG-based moiety. In certain embodiments —P^{a′}, —P^{a″} and —P^{a′″} of formula (a) independently comprise a PEG-based moiety comprising at least 20% PEG, comprising at least 30%, comprising at least 40% PEG, comprising at least 50% PEG, comprising at least 60% PEG, comprising at least 70% PEG, comprising at least 80% PEG or comprising at least 90% PEG.

In certain embodiments —P^{a′}, —P^{a″} and —P^{a′″} of formula (a) independently have a molecular weight ranging from and including 5 kDa to 50 kDa, ranging from and including 5 kDa to 40 kDa, ranging from and including 7.5 kDa to 35 kDa, ranging from and 7.5 to 30 kDa, or ranging from and including 10 to 30 kDa.

In certain embodiments —P^{a′}, P^{a″} and —P^{a′″} of formula (a) have a molecular weight of about 5 kDa.

In certain embodiments —P^{a′}, —P^{a″} and —P^{a′″} of formula (a) have a molecular weight of about 7.5 kDa.

In certain embodiments —P^{a′}, P^{a″} and —P^{a′″} of formula (a) have a molecular weight of about 10 kDa.

In certain embodiments —P^{a′}, —P^{a″} and —P^{a′″} of formula (a) have a molecular weight of about 12.5 kDa.

In certain embodiments —P^{a′}, P^{a″} and —P^{a′″} of formula (a) have a molecular weight of about 15 kDa.

In certain embodiments —P^{a′}, P^{a″} and —P^{a′″} of formula (a) have a molecular weight of about 20 kDa.

In certain embodiments —Z comprises one moiety of formula (a).

In certain embodiments —Z comprises two moieties of formula (a).

In certain embodiments —Z comprises three moieties of formula (a).

In certain embodiments —Z is a moiety of formula (a).

In certain embodiments —Z comprises a moiety of formula (b)

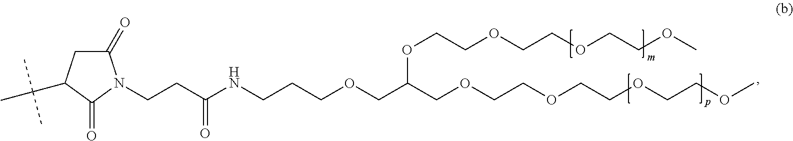

wherein
the dashed line indicates attachment to -L²- or to the remainder of —Z; and
m and p are independently of each other an integer ranging from and including 150 to 1000; preferably an integer ranging from and including 150 to 500; more preferably an integer ranging from and including 200 to 500; and most preferably an integer ranging from and including 400 to 500.

In certain embodiments m and p of formula (b) are the same integer.

In certain embodiments m and p of formula (b) are about 450.

In certain embodiments —Z is a moiety of formula (b).

The total mass of the PTH compound is in certain embodiments at least 10 kDa, such as at least 12 kDa, such as at least 15 kDa, such as at least 20 kDa or such as at least 30 kDa; and its total mass preferably is at most 250 kDa, such as at most 200 kDa, 180 kDa, 150 kDa or 100 kDa. It is understood that no meaningful upper molecular weight limit can be provided in case the PTH compound is water-insoluble.

In certain embodiments the PTH compound is of formula (IIf-i):

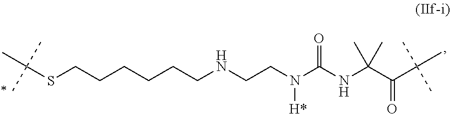

wherein
the unmarked dashed line indicates the attachment to a nitrogen of -D which is a PTH moiety by forming an amide bond; and
the dashed line marked with the asterisk indicates attachment to a moiety

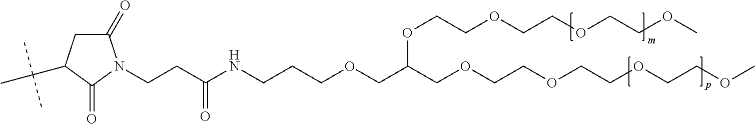

wherein
m and p are independently an integer ranging from and including 400 to 500.

In certain embodiments m and p of formula (IIf-i) are the same integer. In certain embodiments m and p of formula (IIf-i) range from and include 420 to 480. In certain embodiments m and p of formula (IIf-i) range from and include 430 to 470. In certain embodiments m and p of formula (IIf-i) range from and include 440 to 460.

In certain embodiments -D is attached to the PTH prodrug of formula (IIf-i) through the N-terminal amine functional group of the PTH moiety.

In certain embodiments -D of formula (IIf-i) is PTH 1-34, i.e. has the sequence of SEQ ID NO.51.

In certain embodiments the residual activity of the PTH compound is less than 10%, such as less than 1%, such as less than 0.1%, such as less than 0.01%, such as less than 0.001% and in certain embodiments less than 0.0001%.

As used herein the term “residual activity” refers to the activity exhibited by the PTH compound with the PTH moiety bound to a carrier in relation to the activity exhibited by the corresponding free PTH. In this context the term “activity” refers to binding to an activation domain of the PTH/PTHrP1 receptor resulting in activation of adenylate cyclase to generate cAMP, phospholipase C to generate intracellular calcium, or osteoblastic expression of RANKL (which binds to RANK (Receptor Activator of Nuclear Factor kB) on osteoclasts. It is understood that measuring the residual activity of the PTH prodrug for use of the present invention takes time during which a certain amount of PTH will be released from the PTH compound and that such released PTH may distort the results measured for the PTH compound. It is thus accepted practice to test the residual activity of such compound with a conjugate in which the drug moiety, in this case PTH, is non-reversibly, i.e. stably, bound to a carrier, which as closely as possible resembles the structure of the PTH compound for which residual activity is to be measured.

In certain embodiments the PTH compound is administered in the form of a pharmaceutical composition comprising at least one PTH compound as described herein. In certain embodiments such pharmaceutical composition has a pH ranging from and including pH 3 to pH 8. In certain embodiments the pharmaceutical composition has a pH ranging from and including pH 4 to pH 6. In certain embodiments the pharmaceutical composition has a pH ranging from and including pH 4 to pH 5.

In certain embodiments the pharmaceutical composition is a liquid or suspension composition. It is understood that the pharmaceutical composition is a liquid composition if the PTH compound is water-soluble and a suspension formulation if the PTH compound is water-insoluble. In certain embodiments the pharmaceutical composition is a dry formulation which is reconstituted before administration to a patient.

Such liquid, suspension, dry or reconstituted pharmaceutical composition comprises at least one excipient. Excipients used in parenteral formulations may be categorized as, for example, buffering agents, isotonicity modifiers, preservatives, stabilizers, anti-adsorption agents, oxidation protection agents, viscosifiers/viscosity enhancing agents, or other auxiliary agents. However, in some cases, one excipient may have dual or triple functions. In certain embodiments the at least one excipient is selected from the group consisting of
(i) Buffering agents: physiologically tolerated buffers to maintain pH in a desired range, such as sodium phosphate, bicarbonate, succinate, histidine, citrate and acetate, sulphate, nitrate, chloride, pyruvate; antacids such as Mg(OH)₂ or ZnCO₃ may be also used;
(ii) Isotonicity modifiers: to minimize pain that can result from cell damage due to osmotic pressure differences at the injection depot; glycerin and sodium chloride are examples; effective concentrations can be determined by osmometry using an assumed osmolality of 285-315 mOsmol/kg for serum;
(iii) Preservatives and/or antimicrobials: multidose parenteral formulations require the addition of preservatives at a sufficient concentration to minimize risk of patients becoming infected upon injection and corresponding regulatory requirements have been established; typical preservatives include m-cresol, phenol, methylparaben, ethylparaben, propylparaben, butylparaben, chlorobutanol, benzyl alcohol, phenylmercuric nitrate, thimerosol, sorbic acid, potassium sorbate, benzoic acid, chlorocresol, and benzalkonium chloride;
(iv) Stabilizers: Stabilisation is achieved by strengthening of the protein-stabilising forces, by destabilisation of the denatured state, or by direct binding of excipients to the protein; stabilizers may be amino acids such as alanine, arginine, aspartic acid, glycine, histidine, lysine, proline, sugars such as glucose, sucrose, trehalose, polyols such as glycerol, mannitol, sorbitol, salts such as potassium phosphate, sodium sulphate, chelating agents such as EDTA, hexaphosphate, ligands such as divalent metal ions (zinc, calcium, etc.), other salts or organic molecules such as phenolic derivatives; in addition, oligomers or polymers such as cyclodextrins, dextran, dendrimers, PEG or PVP or protamine or HSA may be used;
(v) Anti-adsorption agents: Mainly ionic or non-ionic surfactants or other proteins or soluble polymers are used to coat or adsorb competitively to the inner surface of the formulation's container; e.g., poloxamer (Pluronic F-68), PEG dodecyl ether (Brij 35), polysorbate 20 and 80, dextran, polyethylene glycol, PEG-polyhistidine, BSA and HSA and gelatins; chosen concentration and type of excipient depends on the effect to be avoided but typically a monolayer of surfactant is formed at the interface just above the CMC value;
(vi) Oxidation protection agents: antioxidants such as ascorbic acid, ectoine, methionine, glutathione, monothioglycerol, morin, polyethylenimine (PEI), propyl gallate, and vitamin E; chelating agents such as citric acid, EDTA, hexaphosphate, and thioglycolic acid may also be used;
(vii) Viscosifiers or viscosity enhancers: in case of a suspension retard settling of the particles in the vial and syringe and are used in order to facilitate mixing and resuspension of the particles and to make the suspension easier to inject (i.e., low force on the syringe plunger); suitable viscosifiers or viscosity enhancers are, for example, carbomer viscosifiers like Carbopol 940, Carbopol Ultrez 10, cellulose derivatives like hydroxypropylmethylcellulose (hypromellose, HPMC) or diethylaminoethyl cellulose (DEAE or DEAE-C), colloidal magnesium silicate (Veegum) or sodium silicate, hydroxyapatite gel, tricalcium phosphate gel, xanthans, carrageenans like Satia gum UTC 30, aliphatic poly(hydroxy acids), such as poly(D,L- or L-lactic acid) (PLA) and poly(glycolic acid) (PGA) and their copolymers (PLGA), terpolymers of D,L-lactide, glycolide and caprolactone, poloxamers, hydrophilic poly(oxyethylene) blocks and hydrophobic poly(oxypropylene) blocks to make up a triblock of poly(oxyethylene)-poly(oxypropylene)-poly(oxyethylene) (e.g. Pluronic®), polyetherester copolymer, such as a polyethylene glycol terephthalate/polybutylene terephthalate copolymer, sucrose acetate isobutyrate (SAIB), dextran or derivatives thereof, combinations of dextrans and PEG, polydimethylsiloxane, collagen, chitosan, polyvinyl alcohol (PVA) and derivatives, polyalkylimides, poly (acrylamide-co-diallyldimethyl ammonium (DADMA)), polyvinylpyrrolidone (PVP), glycosaminoglycans (GAGs) such as dermatan sulfate, chondroitin sulfate, keratan sulfate, heparin, heparan sulfate, hyaluronan, ABA triblock or AB block copolymers composed of hydrophobic A-blocks, such as polylactide (PLA) or poly(lactide-co-glycolide) (PLGA), and hydrophilic B-blocks, such as polyethylene glycol (PEG) or polyvinyl pyrrolidone; such block copolymers as well as the abovementioned poloxamers may exhibit reverse thermal gelation behavior (fluid state at room temperature to facilitate administration and gel state above sol-gel transition temperature at body temperature after injection);
(viii) Spreading or diffusing agent: modifies the permeability of connective tissue through the hydrolysis of components of the extracellular matrix in the intrastitial space such as but not limited to hyaluronic acid, a polysaccharide found in the intercellular space of connective tissue; a spreading agent such as but not limited to hyaluronidase temporarily decreases the viscosity of the extracellular matrix and promotes diffusion of injected drugs; and
(ix) Other auxiliary agents: such as wetting agents, viscosity modifiers, antibiotics, hyaluronidase; acids and bases such as hydrochloric acid and sodium hydroxide are auxiliary agents necessary for pH adjustment during manufacture.

The treatment of hyperparathyroidism comprises titrating the patient off of standard of care within four weeks from the time the first dose of the PTH compound was administered.

Various titration schemes are suitable. In certain embodiments titrating the patients off of standard of care involves a stepwise reduction followed by complete omission of orally administered active vitamin D, followed by a stepwise reduction followed by complete omission of orally administered calcium. It is understood that some patient's diets do not allow a sufficient nutritional uptake of calcium (usually considered to be ≤750 mg calcium per day), as may be the case in lactose-intolerant patients, for example. These patients continue taking oral calcium supplements, in the form of for example a once daily oral administration of calcium, such as in the form of a calcium tablet. This calcium supplement is however not related to the treatment of hypoparathyroidism and is common practice also in healthy subjects.

In certain embodiments the titration scheme is as follows (assuming maintenance of a normal serum calcium level and no hypocalcemic symptoms):
(i) Visit 1: Decrease active vitamin D dose by 33-50% (e.g., skip 2^{nd} dose of the day if taking BID, skip last dose of the day if taking TID, or reduce once daily dose of alphacalcidol ≥1.0 μg by ≥0.5 μg);
(ii) Day 3-4: Discontinue active vitamin D;
(iii) Day 6-7: Decrease calcium supplementation by 50%;
(iv) Day 9-10: If daily nutritional calcium exceeds 750 mg/day, discontinue calcium supplements; if daily nutritional calcium intake is <750 mg/day, a calcium supplement to achieve the RDA can be maintained at the discretion of the physician.

In certain embodiments the titration scheme is as follows (assuming maintenance of a normal serum calcium level and no hypocalcemic symptoms):
(i) Visit 1: Decrease active vitamin D dose by 33-50% (e.g., skip 2^{nd} dose of the day if taking BID, skip last dose of the day if taking TID, or reduce once daily dose of alphacalcidol ≥1.0 μg by ≥0.5 μg);
(ii) Day 3-4: Discontinue active vitamin D;
(iii) Day 6-7: If taking calcium ≤2000 mg/day, decrease calcium by ≥50% (≥400 mg/day); if on calcium >2000 mg/day, decrease calcium by ≥800 mg/day;
(iv) Day 9-10: If on calcium ≤2000 mg/day, discontinue* or decrease calcium to ≤500 mg/day (*if dietary calcium <750 mg/day, maintain calcium at 400 or 500 mg/day); if on calcium >2000 mg/day, decrease calcium by ≥800 mg/day.

The dose of the PTH compound for the single daily administration is as disclosed elsewhere herein. It is understood that certain steps of a titration scheme may have to be repeated with a different dose if the initial dose was too high or too low.

Materials

Compound 1 has the following structure:

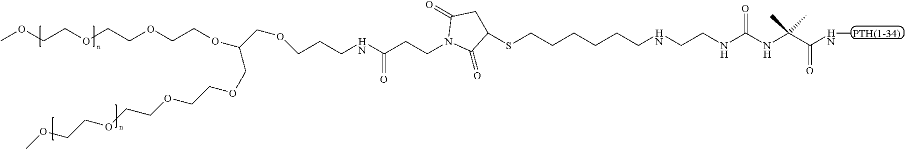

wherein the PTH(1-34) moiety has the sequence of SEQ ID NO:51 and is attached to the remainder of the PTH compound via the nitrogen of the N-terminal amine by forming an amide bond. It is understood that the nitrogen immediately left of “PTH(1-34) corresponds to the nitrogen of the N-terminal amine.

Compound 1 is obtainable from the method described in WO 2018/060312 A1 for compound 18. Compound 1 is also known as “TransCon PTH”.

### EXAMPLE 1

Human participants were randomly assigned to one of four groups: three groups received fixed doses of compound 1 and one group received placebo. Compound 1 or placebo were administered as a subcutaneous injection using a pre-filled injection pen. Neither trial participants nor their doctors knew who were assigned to each group. After the four weeks, participants were eligible to continue in the trial as part of a long-term extension study. During the extension, all participants received compound 1, with the dose adjusted to their individual needs.

The double-blind, placebo-controlled, parallel group treatment period of this trial was designed to enroll approximately 55 male and female adults with either postsurgical HP or autoimmune, genetic, or idiopathic HP for at least 26 weeks, from up to approximately 40 sites worldwide. ClinicalTrials.gov Identifier: NCT04009291 Subjects were randomized into 4 treatment groups (1:1:1:1):
Compound 1 15 μg/day*
Compound 1 18 μg/day*
Compound 1 21 μg/day*
Placebo for compound 1 (excipients solution)
   (*Dose of compound 1 refers to dose of PTH(1-34) administered measured in PTH equivalents)

To maintain blinding, the placebo group were sub-randomized into 3 groups (1:1:1) to mimic doses of 15, 18, and 21 μg/day.

Subjects remained on the same dose of study drug throughout the 4-week Blinded Treatment Period. Following successful completion of the Blinded Treatment Period, subjects entered open-label Extension Period at which time all subjects received compound 1.

The entire study was designed such that each subject's participation may last up to 58 weeks plus a screening period of up to approximately 4 weeks.
Screening Period (supplement optimization): Up to approximately 4 weeks
Blinded Treatment Period (compound 1 dose stable with SOC optimization): 4 weeks
Extension Period (open-label compound 1 treatment): 54 weeks, with up to an initial 14 weeks of compound 1 titration and SOC optimization, followed by approximately 40 weeks of stable dosing

Titration Scheme (Assuming Maintenance of a Normal Serum Calcium Level)
Visit 1: Decrease active vitamin D dose by 33-50% (eg, skip 2^{nd} dose of the day if taking BID, skip last dose of the day if taking TID, or reduce once daily dose of alphacalcidol ≥1.0 μg by ≥0.5 μg);
Day 3-4: Discontinue active vitamin D;
Day 6-7: If on active vitamin D, discontinue active vitamin D; if off active vitamin D, and on calcium ≤2000 mg/day, decrease calcium by ≥50% (≥400 mg/day) and if on calcium >2000 mg/day, decrease calcium by ≥800 mg/day;
Day 9-10: If on active vitamin D, discontinue active vitamin D; if off active vitamin D, and on calcium ≤2000 mg/day, discontinue* or decrease calcium to ≤500 mg/day (*if dietary calcium <750 mg/day, maintain calcium at 400 or 500 mg/day); if on calcium >2000 mg/day, decrease calcium by ≥800 mg/day

Results

Preliminary data on first 8 subjects completing 4 weeks follow-up in open label extension demonstrated that all subjects are completely off standard of care with 8 of 8 subjects no longer require active vitamin D. 7/8 subjects no longer required calcium supplementation. One subject continued taking a daily dietary supplemental dose of <500 mg calcium in order to reach the recommended daily intake for calcium.

Full data, 4-weeks fixed dose period: All 59 subjects from the trial completed an initial fixed dose 4-week period, where optimization of doses was not allowed. Patients randomized to compound 1 were able to discontinue oral active vitamin D. Similarly, these patients were able to stop therapeutic doses of oral calcium, and oral calcium intake was reduced from a mean of 2213 mg/day at baseline to a mean of 560 mg/day after 4 weeks of dosing with compound 1. In contrast, patients receiving placebo could not discontinue standard of care, and had a reduction of oral active vitamin D from 1.1 ug/day at baseline to 0.9 ug/day at 4 weeks, and reduced oral calcium supplements from 1685 mg/day at baseline to 1368 mg/day at 4 weeks.

Full data, 26 weeks: All 59 subjects completed the initial 4-week period and continued in the open label extension (OLE); 58 subjects continued in the OLE beyond 6 months (1 withdrew unrelated to safety or efficacy). Patients treated with compound 1 continued to reduce oral calcium intake, which dropped to a mean of 294 mg/day at week 26. Furthermore, mean 24-hour uCa decreased from a baseline mean of 415 mg/24 h to 178 mg/24 h by Week 26 (n=44) while maintaining normal sCa and reducing sP and CaxP. Importanly, no subjects had PTH treatment-emergent adverse events related to hyper- or hypocalcemia leading to ER/urgent care visit and/or hospitalization.

### EXAMPLE 2

Administration of compound 1 to the human participants described in example 1 resulted in a statistically significant improvement compared to placebo in a double blinded trial for the SF-36. The SF-36 survey consists of 36 questions and the results are summarized in a Physical Component Summary (PCS) and Mental Component Summary (MCS). At baseline, all subjects had lower-than-average SF-36 scores. Statistically significant and clinically meaningful improvements in PCS and MCS were noted in the 4 weeks double blinded controlled part of the phase 2 trial. For the PCS score, using a normative scoring system with a score of 50 as the norm for the general population and an ANCOVA model, subjects receiving compound 1 demonstrated a mean 4.5 point increase compared to a mean -0.69 point decrease for placebo. The placebo adjusted mean difference is 5.2 points with a p-value of 0.013. The minimally important difference for PCS is 2 points.

For the Mental Component Summary score, subjects receiving compound 1 demonstrated a mean 6.0-point increase compared to a mean -3.8 point decrease for placebo. The placebo adjusted difference in mean of 9.8 points with a p-value of 0.0003. The minimally important difference for MCS is 3 points.

Full data, 26 weeks: All 59 subjects completed the initial 4-week period and continued in the open label extension (OLE); 58 subjects continue in the OLE beyond 6 months (1 withdrew unrelated to safety or efficacy). The mean scores for all SF-36 summary and domains increased from below normal at baseline to within the normal range by week 26. The HPES Symptom and Impact scores continuously improved through 26 weeks for patients receiving compound 1 and placebo subjects switching to compound 1. Details are shown in Table 1. Compound 1 continued to be well-tolerated with no treatment-related serious or severe adverse events.

TABLE 1
Mean scores for all SF-36 domains

| | | | Placebo | | | | | |
|---|---|---|---|---|---|---|---|---|
| | Placebo | | Switch to | Compound 1 | | | All compound 1 | |
| | (n = 15) | | compound 1 | (n = 44) | | | (n = 59) | |
| SF-36 | | Week | (n = 15) | | Week | 6 | | 6 |
| domain | Baseline | 4 | 6 Months | Baseline | 4 | Months | Baseline | Months |
| PF | 45 (11) | 46 (14) | 51 (7) | 46 (9) | 51 (6) | 52 (5) | 46 (10) | 51 (6) |
| RP | 42 (10) | 42 (14) | 49 (11) | 42 (10) | 49 (8) | 51 (6) | 42 (10) | 50 (7) |
| BP | 43 (11) | 40 (16) | 46 (10) | 46 (10) | 49 (8) | 51 (9) | 45 (10) | 50 (9) |
| GH | 44 (10) | 47 (11) | 50 (7) | 43 (10) | 47 (8) | 51 (9) | 43 (10) | 51 (8) |
| VT | 44 (12) | 43 (12) | 52 (10) | 42 (11) | 49 (9) | 53 (8) | 43 (11) | 53 (8) |
| SF | 44 (11) | 41 (15) | 53 (5) | 42 (10) | 50 (8) | 52 (6) | 43 (10) | 52 (6) |
| RE | 45 (12) | 39 (16) | 51 (7) | 42 (13) | 49 (10) | 50 (8) | 43 (13) | 50 (7) |
| MH | 47 (9) | 47 (11) | 55 (5) | 46 (9) | 51 (8) | 51 (8) | 46 (9) | 52 (7) |
| PCS | 43 (12) | 44 (14) | 48 (8) | 45 (10) | 49 (7) | 51 (7) | 44 (11) | 50 (8) |
| MCS | 46 (10) | 43 (12) | 54 (6) | 43 (11) | 50 (9) | 51 (8) | 44 (11) | 52 (8) |

PF = physical functioning;
RP = physical role functioning;
BP = bodily pain;
GH = general health perceptions;
VT = vitality;
SF = social role functioning;
RE = emotional role functioning;
MH = mental health;
PCS = Physical Component Summary;
MCS = Mental Component Summary

### EXAMPLE 3

All 59 subjects completed the initial 4-week period and continued in the open label extension (OLE); 58 subjects continue in the OLE beyond 6 months (1 withdrew unrelated to safety or efficacy). At baseline, mean BMD Z-scores at lumbar spine, femoral neck and total hip were elevated due to lack of bone turnover. With treatment with compound 1, BMD mean Z-score trended toward normalization at week 26, as shown in Table 2.

TABLE 2
Bone mineral density measurements

| | | | |
|---|---|---|---|
| | | | Week 26 change from |
| N = 44 | Baseline | Week 26 | baseline |

Lumbar spine L1-L4

| | | | |
|---|---|---|---|
| Mean BMD Z-score | 1.6 | 0.9 | −0.7 |

Femoral neck

| | | | |
|---|---|---|---|
| Mean BMD Z-score | 1.2 | 0.7 | −0.5 |

Total hip

| | | | |
|---|---|---|---|
| Mean BMD Z-score | 1.0 | 0.6 | −0.5 |

⅓ radius

| | | | |
|---|---|---|---|
| Mean BMD Z-score | 0.4 | 0.4 | 0.0 |

### Abbreviations

BID bis in die, i.e. twice a day
HP hypoparathyroidism
PTH parathyroid hormone
RDA recommended daily/dietary allowance
sCA serum calcium
SOC standard-of-care
TID ter in die, i.e three times a day

1. A method of treating or controlling a patient suffering from hypoparathyroidism, wherein the method comprises the step of administering to the patient single daily administrations of a PTH compound and titrating the patient off a standard of care within four weeks from the time the first dose of the PTH compound was administered.

2. The method of claim 1, wherein the patient is titrated off the standard of care within three weeks from the time the first dose of the PTH compound is administered.

3. The method of claim 1, wherein the patient is titrated off the standard of care within two weeks from the time the first dose of the PTH compound is administered.

4. The method of claim 1, wherein the single daily dose of the PTH compound is below 31 μg/day.

5. The method of claim 1, wherein the single daily dose of the PTH compound is selected from 15 μg/day, 18 μg/day and 21 μg/day.

6. The method of claim 1, wherein the patient is a human patient.

7. The method of claim 1, wherein administration is by subcutaneous injection.

8. The method of claim 1, wherein administration is with a pen injector.

9. The method of claim 1, wherein titrating the patient off the standard of care is performed according to the following the titration scheme
(i) Visit 1: Decrease active vitamin D dose by 33-50%;
(ii) Day 3-4: Discontinue active vitamin D;
(iii) Day 6-7: Decrease calcium supplementation by 50%;
(iv) Day 9-10: If daily nutritional calcium exceeds 750 mg/day, discontinue calcium supplements; if daily nutritional calcium intake is <750 mg/day, a calcium supplement to achieve the RDA can be maintained at the discretion of the physician.

10. The method of claim 1, wherein titrating the patient off the standard of care is performed according to the following the titration scheme
(i) Visit 1: Decrease active vitamin D dose by 33-50%
(ii) Day 3-4: Discontinue active vitamin D;
(iii) Day 6-7: If taking calcium ≤2000 mg/day, decrease calcium by ≥50% (≥400 mg/day); if on calcium >2000 mg/day, decrease calcium by ≥800 mg/day;
(iv) Day 9-10: If on calcium ≤2000 mg/day discontinue; if dietary calcium <750 mg/day maintain calcium at 400 or 500 mg/day or decrease calcium to 500 mg/day; if on calcium >2000 mg/day, decrease calcium by ≥800 mg/day.

11. The method of claim 1, wherein 4 weeks after administration of the first dose of the PTH compound a statistically significant change in the Short Form-36 Physical Component Summary, in the SF-36 Mental Component Summary or both the SF-36 PCS and SF-36 MCS is achieved.

12. The method of claim 1, wherein the PTH compound is a conjugate or a pharmaceutically acceptable salt thereof comprising at least one moiety -D conjugated via at least one moiety -L¹-L²- to at least one moiety Z, wherein the linkage between -D and -L¹- is reversible and wherein a moiety -L²- is conjugated to Z, wherein each -D is independently a PTH moiety; each -L¹- is independently a reversible linker moiety; each -L²- is independently a single chemical bond or a spacer moiety; and each Z is independently a polymeric moiety or a C₈₋₂₄ alkyl moiety.

13. The method of claim 1, wherein the PTH compound comprises a PTH moiety having the sequence of SEQ ID NO:51.

14. The method of claim 1, wherein the PTH compound is a compound of formula (Ia) or (Ib) or a pharmaceutically acceptable salt thereof 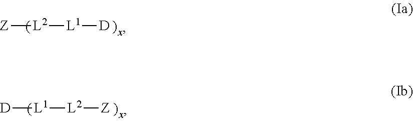
wherein
-D is a PTH moiety;
-L¹- is a linker moiety reversibly and covalently connected to the PTH moiety -D through a functional group of PTH;
-L²- is a single chemical bond or a spacer moiety;
—Z is a polymer moiety or a C₈₋₂₄ alkyl moiety;
x is an integer selected from the group consisting of 1, 2, 3, 4, 5, 6, 7, 8, 9, 10, 11, 12, 13, 14, 15 or 16; and
y is an integer selected from the group consisting of 2, 3, 4 and 5.

15. The method of claim 12, wherein the moiety -L¹-L²- is selected from the group consisting of 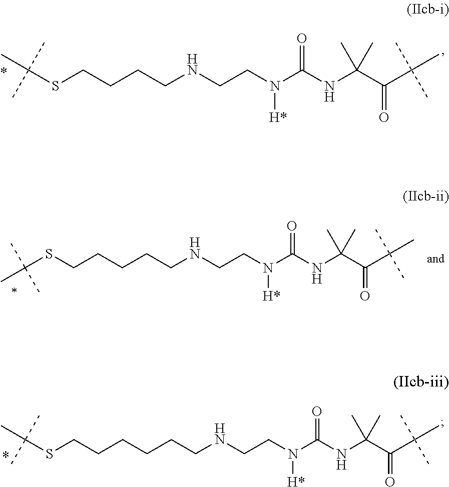
wherein
the unmarked dashed line indicates the attachment to a nitrogen of -D which is a PTH moiety by forming an amide bond; and
the dashed line marked with the asterisk indicates attachment to —Z.

16. The method of claim 12, wherein the PTH compound is of formula (IIf-i): 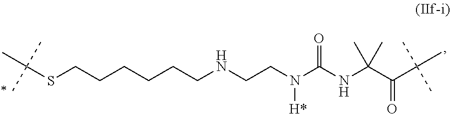
wherein
the unmarked dashed line indicates the attachment to a nitrogen of -D which is a PTH moiety by forming an amide bond; and
the dashed line marked with the asterisk indicates attachment to a moiety 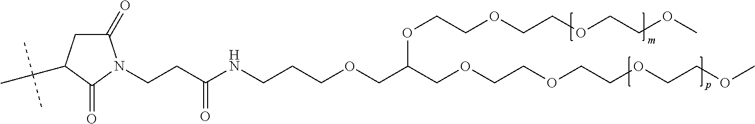
wherein
m and p are independently an integer ranging from and including 400 to 500.

17. The method of claim 1, wherein the PTH compound comprises a PTH moiety having the sequence SEQ ID NO:47, SEQ ID NO:48, SEQ ID NO:49, SEQ ID NO:50, SEQ ID NO:51, SEQ ID NO:52, SEQ ID NO:53, SEQ ID NO:54, SEQ ID NO:55, SEQ ID NO:107, SEQ ID NO:108, SEQ ID NO:109, SEQ ID NO:110, SEQ ID NO:111, SEQ ID NO:112, SEQ ID NO:113, SEQ ID NO:114 or SEQ ID NO:115.

18. The method of claim 12, wherein —Z is a PEG-based polymer.

19. The method of claim 12, wherein —Z is a branched PEG-based polymer.
